(19) United States
(12) Patent Application Publication
(10) Pub. No.: US 2019/0156919 A1
(43) Pub. Date: May 23, 2019

(54) DETERMINING RELATIONSHIPS BETWEEN RISKS FOR BIOLOGICAL CONDITIONS AND DYNAMIC ANALYTES

(71) Applicants: ARIVALE, INC., Seattle, WA (US); INSTITUTE FOR SYSTEMS BIOLOGY, Seattle, WA (US)

(72) Inventors: Andrew Tyler Magis, Seattle, WA (US); John Carl Earls, Chapin, IL (US); Nathan Price, Seattle, WA (US)

(73) Assignees: ARIVALE, INC., Seattle, WA (US); INSTITUTE FOR SYSTEMS BIOLOGY, Seattle, WA (US)

(21) Appl. No.: 16/215,723

(22) Filed: Dec. 11, 2018

Related U.S. Application Data
(60) Provisional application No. 62/423,386, filed on Nov. 17, 2016.

(51) Int. Cl. G16B 40/00; G16B 20/40; G16B 25/10; G16B 50/00

(52) U.S. Cl. CPC G16B 40/00; G16B 50/00; G16B 25/10; G16B 20/40

(57) ABSTRACT

The present disclosure describes systems and methods to elucidate unknown relationships and interactions between and among complex biological systems and components thereof. The systems and methods can inform clinical interventions in individuals before phenotypes of an adverse condition emerge.

### CROSS REFERENCE TO RELATED APPLICATION

This application is a continuation of International Patent Application No. PCT/US2017/062290, filed Nov. 17, 2017, which claims priority to U.S. Provisional Patent Application No. 62/423,386, filed Nov. 17, 2016 which is incorporated by reference herein in its entirety.

### FIELD OF THE DISCLOSURE

The present disclosure provides systems and methods to elucidate unknown relationships and interactions between and among complex biological systems and components thereof. The systems and methods can inform clinical interventions in individuals before phenotypes of an adverse condition emerge, thus preserving scientific wellness in individuals.

### BACKGROUND OF THE DISCLOSURE

The increase in available genetic data provided by individual genome sequencing has led to an increased effort in identifying associations between human genetic variation, physiology, and condition (e.g., disease) risk. Individual genome information may be used to predict risk for serious conditions based solely on genetic variations associated with various conditions. However, past data has shown that genetic predisposition for a particular condition alone is of limited predictive value for an individual developing that condition. In recent years, several quantified-self studies have begun to show the power of data-intensive longitudinal analysis for individuals highly motivated to examine personal health data for signs of reversible early disease or disease risk factors (Chen, et al., (2012). Cell 148, 1293-1307.; David, et al., (2014). Genome Biol. 15, R89., 2014; Smarr (2012). Biotechnol J 7, 980-991).

### SUMMARY OF THE DISCLOSURE

With the goal of laying a foundation for personalized healthcare and scientific wellness, longitudinal data for a large number of individuals was collected and analyzed using efficiency-increasing systems and methods. Each individual's dataset included genomic data, and measurements of dynamic analytes through measurement of clinical tests, microbiomes, metabolomes, and proteomes. The number of individuals in the analysis combined with the efficiency-increasing systems and methods and the diverse and dense measures led to numerous unanticipated findings with novel applications in the new realm of clinical medicine that is predictive, preventive, personalized, and participatory (P4 medicine).

### BRIEF DESCRIPTION OF THE FIGURES

FIG. 1 is a pictorial diagram illustrating an environment in which clinical testing data and biological data of individuals can be used to determine correlations between biological indicators.

FIG. 2 is a pictorial diagram illustrating an example environment in which multi-omic longitudinal data analysis can be implemented.

FIG. 3 is a block diagram illustrating select components of a data correlation identification system.

FIG. 4 is a graph of modularity vs community analysis iteration. The maximum modularity observed in the inter-omic community analysis was 0.386 at iteration 61 of community pruning. There were 267 total iterations of community analysis.

FIG. 5 is a flow diagram of an example method for identifying dynamic analytes associated with condition onset that are independent of biochemical products of a known genetic risk set.

FIG. 6 is a flow diagram of an example method for identifying subjects appropriate for a preventative measure based on genetic risk and expression of an independent dynamic analyte.

FIG. 7 is a flow diagram of an example method for determining whether a change in a dynamic analyte precedes or follows onset of a condition.

FIG. 8 is a flow diagram of an example method to analyze multi-omic data and genetic risk scores to identify information about relationships between biological conditions and dynamic analytes.

FIG. 9 is a flow diagram of an example method to produce a network of correlations based on biological data and/or clinical testing data and analyzing the network of correlations to identify information about relationships between biological conditions and dynamic analytes.

FIG. 10 shows gut microbiome stability over nine months. Participant microbiomes tend to resemble themselves over time. Plotted in grey is the unweighted UniFrac distance between consecutive microbiome samples for all participants. The box-and-whisker plots represent the distance distribution between each sample and all others in the same time points. In 97% of cases, an individual's cross-timepoint distance is lower than the median inter-individual distance.

FIG. 11 shows a cross-correlation network with statistically-significant Spearman correlations (p_{adj}<0.05) between all datasets collected in the cohort.

FIG. 12 shows a cross-correlation network for a cardiometabolic community. The lines indicate significant (p_{adj}<0.05) correlations between vertices and edges of the cardiometabolic community.

FIGS. 13 and 14A-14D show correlations between additional communities: (13) Serotonin community (14A) Cholesterol community (14B) α-diversity community (14C) The genetic risk for inflammatory bowel disease is negatively correlated with cystine (14D) The genetic risk for bladder cancer is positively correlated with 5-acetylamino-6-formylamino-3-methyluracil (AFMU).

FIGS. 15A, 15B show cumulative genetic risk being predictive of LDL-C levels: (15A) OLS regression on the dependent variable LDL-C, (15B) Spearman correlations between actual LDL-C and predicted LDL-C based on three OLS models, while excluding participants on cholesterol-lowering medication (N=77). Spearman's p between predicted and actual LDL-C is also shown.

FIGS. 16A-16D show cumulative genetic risk correlates with blood analytes. Spearman correlations between polygenic scores (x-axis) and analyte measurements (y-axis) from the correlation network: (16A) dihomo-γ-linoleic acid polygenic score vs. dihomo-γ-linoleic acid, (16B) bilirubin polygenic score vs. bilirubin polygenic score, (16C) inflammatory bowel disease polygenic score vs. inflammatory bowel disease polygenic score, (16D) bladder cancer polygenic score vs. 5-acetylamino-6-formylamino-3-methyluracil.

FIGS. 17 and 18 show reproducibility across different vendors. Several proteins and metabolites were measured by multiple vendors. Shown in FIGS. 15 and 16 are the Spearman correlations between these repeated measurements, sorted in descending order by rho.

FIG. 19 shows response to vitamin D supplementation. This figure shows the change in vitamin D levels between the baseline and 3-month blood draw.

FIGS. 20A-20C shows breadth of data collected longitudinally on 108 individuals and correlations across data types: (20A) Timeline of important events in the P100. (20B) Schematic of the data collected every three months throughout the study. (20C) Subset of top statistically-significant Spearman cross-sectional correlations between all datasets collected in the cohort. Each line represents one correlation. Up to 100 correlations per pair of data types are shown.

FIGS. 21A (males), 21B (females) show genetic risk factors for hemochromatosis with Boxplots for ferritin levels of the male and female participants by round being shown.

FIG. 22 shows MMP2 levels according to quintiles of genetic risk for Alzheimer's disease relative to age for a population of individuals that were not diagnosed with Alzheimer's disease.

### DETAILED DESCRIPTION

The increase in available genetic data provided by individual genome sequencing has led to an increased effort in identifying associations between human genetic variation, physiology, and biological condition propensity, such as propensity for disease. Individual genome information may be used to predict risk for serious biological conditions based solely on genetic variations associated with various biological conditions. However, past data has shown that genetic predisposition for a particular condition alone is of limited predictive value for an individual developing that condition. In recent years, several quantified-self studies have begun to show the power of data-intensive longitudinal analysis for individuals highly motivated to examine personal health data for signs of reversible early biological condition onset or biological condition risk factors (Chen, et al., (2012). Cell 148, 1293-1307; David, et al., (2014). Genome Biol. 15, R89; Smarr (2012). Biotechnol J 7, 980-991).

With the goal of laying a foundation for personalized healthcare and scientific wellness, longitudinal data for a large number of individuals was collected. Each individual's dataset included genomic data, and measurements of dynamic analytes through measure of clinical tests, microbiomes, metabolomes, and proteomes. The number of individuals in the analysis combined with the diverse and dense measures led to numerous unanticipated findings with novel applications in the new realm of clinical medicine that is predictive, preventive, personalized, and participatory (P4 medicine).

In particular embodiments, the systems and methods can be used to identify unanticipated correlations between genetic risk for a biological condition and a dynamic analyte before a biological condition (e.g., disease) phenotype emerges.

In particular embodiments, the systems and methods can be used to identify dynamic analytes that are more strongly correlated with a biological condition or genetic risk than previously identified dynamic analytes.

In particular embodiments, the systems and methods can be used to uncover unknown correlations between the genome, dynamic analytes and/or environmental factors to further the understanding of complex interactions between biological systems and components thereof.

In particular embodiments, the systems and methods can be used to clarify whether a dynamic analyte associated with a condition precedes development of the biological condition or is a result of the biological condition's development.

In particular embodiments, the systems and methods can be used to identify individuals with a genetic risk for a biological condition and determine if the individual would likely benefit from an intervention based on the level of a dynamic analyte.

In particular embodiments, the systems and methods can be used to identify individuals for an intervention based on a change in the level of a dynamic analyte before phenotypic traits of a biological condition emerge. In some examples, the individual can have a genetic risk for the biological condition. Additionally, or alternatively, the dynamic analyte can have an unanticipated correlation with the genetic risk.

The techniques and embodiments described herein provide efficient methods to determine relationships between risks for biological conditions and dynamic analytes. The techniques and embodiments described herein can also minimize the processing resources and memory resources utilized to determine relationships between risks for biological conditions and dynamic analytes. Particular embodiments described herein provide technical improvements over conventional systems by implementing various techniques, combinations of techniques, and refinements of techniques that identify relationships between biological conditions and dynamic analytes. For example, when analyzing a body of data, such as clinical testing data, polygenic scores, and/or values of dynamic analytes for particular individuals, the body of data can be filtered before being analyzed to determine relationships between biological conditions and dynamic analytes. That is, certain criteria can be applied to the body of data or to portions of the body of data to identify a subset of the body of data to be analyzed to determine relationships between biological conditions and dynamic analytes. By filtering the body of data according to particular criteria, the relationships between biological conditions and dynamic analytes can be more accurately determined than if the filtering had not been performed and can be efficiently determined with respect to reducing the number of computing resources utilized to determine relationships between biological conditions and dynamic analytes because the content of the data can be more efficiently analyzed. Additionally, the specific computational techniques and combinations of computational techniques utilized to analyze data to determine the relationships between biological conditions and dynamic analytes result in accurate and efficient determinations of relationships between biological conditions and dynamic analytes. In some cases, customized code has been implemented that results in the accurate and efficient determinations of relationships between biological conditions and dynamic analytes. In a particular illustrative example, Spearman's p is used instead of Pearson's r to more accurately and efficiently determine the relationships between biological conditions and dynamic analytes.

Additionally, particular embodiments described herein provide improvements to the field of identifying relationships between biological conditions and dynamic analytes. For example, particular embodiments described herein can identify relationships between biological conditions and dynamic analytes that have not been previously identified. The previously unidentified relationships between biological conditions and dynamic analytes that are identified by embodiments described herein are often not obvious or predictable based on a current body of research. Furthermore, the techniques described herein can provide previously unknown information regarding whether a dynamic analyte and its presence or concentration precede development of a biological condition or is a result of the biological condition having already developed. That is, particular embodiments disclosed herein can determine whether individuals predisposed to a biological condition have a dynamic analyte or a threshold amount of the dynamic analyte or whether individuals that are already symptomatic of the biological condition have the dynamic analyte or a threshold amount of the dynamic analyte. Thus, implementing embodiments described herein can advance the knowledge in the field of identifying relationships between biological conditions and dynamic analytes.

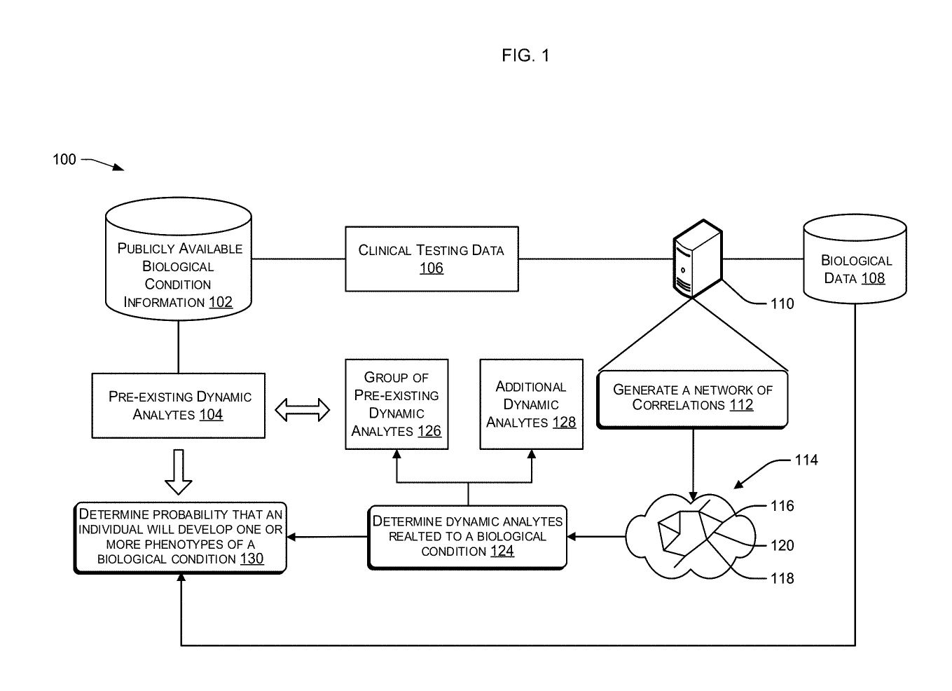

FIG. 1 is a pictorial diagram illustrating an environment 100 in which clinical testing data and biological data of individuals can be used to determine correlations between biological conditions and dynamic analytes. The environment 100 can include a data store that stores publicly available biological condition information 102. The publicly available biological condition information 102 can include research papers, website content, journals, conference proceedings, presentations, other sources of publicly available biological condition information, or combinations thereof. In some embodiments, the publicly available biological condition information 102 can include one or more genome wide association studies (GWAS). The publicly available biological condition information 102 may indicate some correlations between biological conditions and dynamic analytes. In some embodiments, the biological conditions can include diseases. In the illustrative example of FIG. 1, the publicly available biological condition information 102 includes pre-existing dynamic analytes 104. In various examples, the pre-existing dynamic analytes 104 can include one or more metabolites that are correlated with a biological condition. In other examples, the pre-existing dynamic analytes 104 can include one or more proteins that are correlated with a biological condition. In additional examples, the pre-existing dynamic analytes 104 can include one or more portions of a genome that are correlated with a biological condition. In further examples, the pre-existing dynamic analytes 104 can include one or more portions of a microbiome that are correlated with a biological condition.

The publicly available biological condition information 102 can also include clinical testing data 106. The clinical testing data 106 can include results of a number of clinical tests that were performed on subjects. The subjects can include humans, in some cases. In other instances, the subjects can include other mammals. The clinical testing data 106 can also include the methods used to conduct experiments that produced the results of the clinical tests. The clinical testing data 106 can indicate one or more biological indicators that correspond with one or more biological conditions.

The environment 100 can also include an additional data store that stores biological data 108. The biological data 108 can include biological information associated with a group of individuals. In various embodiments, the biological data 108 can include genomic data for a group of individuals. The biological data 108 can also include metabolomics data for a group of individuals. Additionally, the biological data 108 can include proteomic data for a group of individuals. Further, the biological data 108 can include microbiome data for a group of individuals. In particular embodiments, the biological data 108 can include transcriptomic data, epigenetic data, quantified-self data, or any biological data which can be represented as a quantitative measurement. The biological data 108 can be obtained using one or more techniques to collect biological material from the group of individuals, such as blood samples, plasma samples, tissue samples, fecal samples, saliva samples, hair samples, urine samples, combinations thereof, and the like. The biological data 108 can include results produced by performing one or more analyses on the biological material obtained from the group of individuals. In some embodiments, the biological data 108 can be stored in a relational database. In illustrative embodiments, the biological data 108 can be stored using Pandas data frames. In situations where Pandas data frames are used, the biological data 108 can be stored according to the Python programming language. In particular embodiments, the biological data 108 can be stored using R data frames. In situations where R data frames are used, the biological data 108 can be stored according to the R programming language. The use of Pandas data frames or R data frames can increase the efficiency of a system to store and extract the publicly available biological condition information 102 and/or the biological data 108.

The environment 100 can include one or more computing devices 110. The one or more computing devices 110 can include one or more server computing devices, one or more desktop computing devices, one or more laptop computing devices, a cloud computing architecture, or combinations thereof. The one or more computing devices 110 can access the data stores storing the publicly available biological condition information 102 and the biological data 108. It should be noted that the publicly available biological condition information 102 and the biological data 108 can be stored in separate data stores or in the same data store. Additionally, the publicly available biological condition information 102 and the biological data 108 can be stored in one or more distributed storage environments.

The one or more computing devices 110 can obtain at least a portion of the clinical testing data 106, at least a portion of the biological data 108, or both, and perform operation 112 of generating a network of correlations. An example network of correlations 114 is shown in FIG. 1. The network of correlations 114 can include a number of vertices and a number of edges. For example, the network of correlations 114 can include a first vertex 116, a second vertex 118, and an edge 120 formed between the first vertex 116 and the second vertex 118. The vertices of the network of correlations 114 can correspond to a biological indicator or a clinical test. The edges of the network of correlations 114 can indicate a correlation between two vertices. In some embodiments, the edges of the network of correlations 114 can indicate a correlation between two vertices that is at least a threshold level. In some embodiments, the edge between two vertices can be calculated using the algorithm of Spearman. In some embodiments, the edge between two vertices can be calculated using the algorithm of Kruskal or Pearson. In some embodiments, the edge between two vertices can be computed using a statistical method that determines a relationship between variables, such as linear regression, nonlinear regression, or mutual information.

Using the network of correlations 114, the one or more computing devices 110 or other computing devices can perform an operation 124 of determining biological indicators of a biological condition. In some embodiments, the operation 124 can determine that levels of one or more proteins may correspond with a particular biological condition. Also, the operation 124 can determine that levels of one or more metabolites can correspond with a particular biological condition. Additionally, the operation 124 can determine that the presence of certain portions of a genome can correspond with a particular biological condition. Further, the operation 124 can determine that the presence of certain portions of a microbiome can correspond with a particular biological condition. The operation 124 can also determine that the presence of certain portions of the transcriptome can correspond with a particular biological condition. Further, the operation 124 can determine that the presence of certain portions of the epigenome can correspond with a particular condition. In addition, the operation 124 can determine that the presence of certain quantified-self data can correspond with a particular condition. In an illustrative example, the operation 124 can determine that biological indicators such as C-peptide, triglycerides, insulin, fasting glucose, high density lipoprotein (HDL) cholesterol, small low-density lipoprotein (LDL) particle number, and/or homeostatic model assessment-insulin resistance (HOMA-IR) correspond with cardiometabolic health.

The operation 124 can produce a group of pre-existing dynamic analytes 126 that correspond with at least a portion of the pre-existing dynamic analytes 104 that have been previously determined. In this way, the correlations produced by the environment 100 can be verified by previously determined correlations between biological indicators and dynamic analytes. Additionally, the correlations produced by the environment 100 can be used to verify the previously determined correlations between biological indicators and dynamic analytes. The operation 124 can also produce additional dynamic analytes 128. The additional dynamic analytes 128 can include dynamic analytes that have not previously been associated with a particular biological condition. Thus, the correlations produced by the environment 100 can expand the level of scientific knowledge with respect to dynamic analytes that can correlate with particular biological conditions. In some cases, the additional dynamic analytes 128 can be used to implement clinical studies to explore the effects of the additional biological indicators 128 on the biological conditions that the additional biological indicators 128 have been correlated with as indicated by a network of correlations, such as the network of correlations 114. The additional biological indicators 128 can also be used to produce recommendations for interventions that can help reduce the probability of individuals exhibiting one or more phenotypes of a biological condition. In an illustrative embodiment, the operation 124 can utilize the network of correlations 114 to determine that inhibin beta C chain (INHBC) is correlated with cardiovascular risk even though INHBC is not currently characterized as being an indicator for cardiovascular risk in the publicly available biological condition information 102 as of the date of filing this patent application.

In the illustrative example of FIG. 1, the pre-existing biological indicators 104 and/or the additional biological indicators 128 can be analyzed in conjunction with the biological data 108 of individuals to perform operation 130 of determining a probability that an individual can develop one or more phenotypes of a biological condition. The probability that an individual can develop one or more phenotypes of a biological condition can be based at least partly on levels of dynamic analytes obtained from the biological data 108. The probability that an individual can develop one or more phenotypes of a biological condition can be based at least partly on the presence in an individual of at least a portion of a genome and/or at least a portion of a microbiome that is correlated with the biological condition. In some embodiments, the probability that an individual can develop one or more phenotypes of a biological condition can be based at least partly on the presence in an individual of particular metabolites, proteins, transcripts, epigenetic markers, quantified-self measurements, combinations thereof, or any dynamic analyte data or combination of dynamic analyte data which can be represented as a quantitative measurement. The data for individuals used to determine a probability that an individual can develop one or more phenotypes of a biological condition can be obtained from the biological data 108.

Based at least partly on the probability that an individual can develop one or more phenotypes for a biological condition, a plan can be developed to reduce the probability that the individual can develop one or more phenotypes of the biological condition. The plan can include aspects of health and wellness that have been shown to reduce the probability that an individual will develop one or more phenotypes of the biological condition, such as diet, exercise, nutritional supplements, combinations thereof, and so forth.

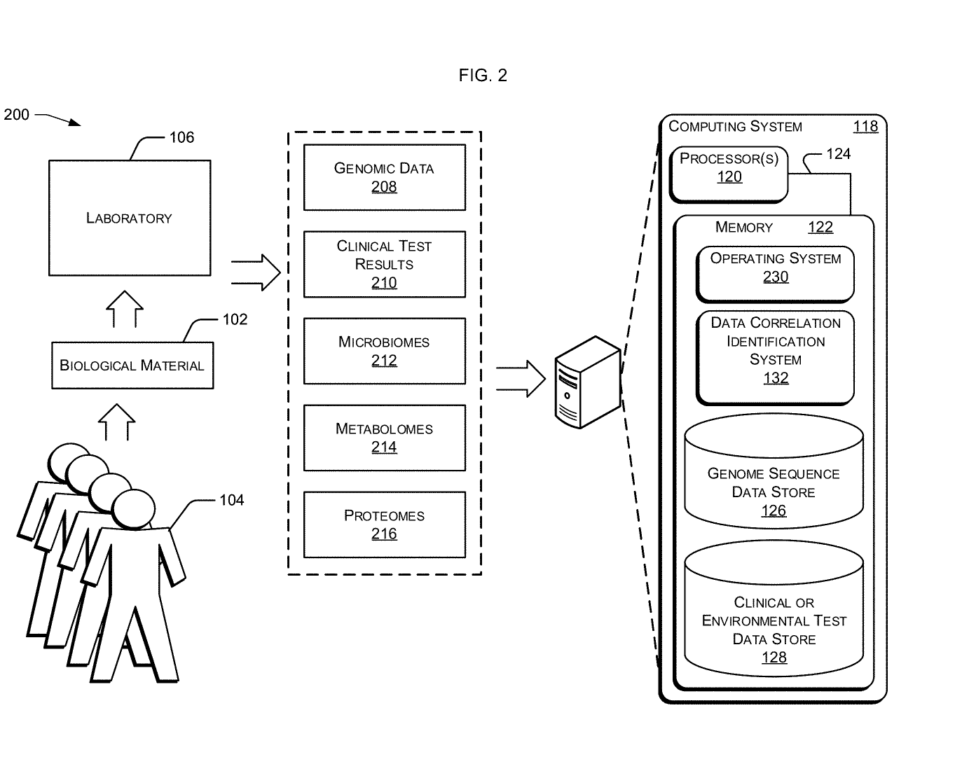

FIG. 2 illustrates an example environment 200 in which multi-omic longitudinal data analysis can be implemented. Multi-omic longitudinal data refers to at least two of genomic data, dynamic analyte data, and/or environmental data collected for at least 2 points in time.

In the illustrated example, biological material 202 is obtained from a number of individuals 204, for example, in a medical laboratory 206. Examples of the biological material 202 that may be obtained may include blood, urine, stool, hair, skin, and/or saliva samples. The collected biological material 202 for each individual 204 can be analyzed in the laboratory 206 to generate genomic data 208 for each individual 204, as well as data representing biological indicators that can include clinical or environmental test results 210 and/or one or more dynamic analytes (e.g., microbiomes 212, metabolomes 214, proteomes 216, and/or other analytes).

In particular embodiments, genomic data 208 includes genetic sequence data and/or whole genome sequencing data. Genetic sequence data can include complete or incomplete chromosomal and/or mitochondrial genetic sequence information, which can be derived from DNA or RNA samples obtained from an individual 204. Genetic sequence information can be obtained for genes (regions of DNA that code for functional RNAs and/or proteins), and also for intergenic regions (regions of DNA between genes). For whole genome sequencing, a total non-redundant sequence information for an individual 204 can be obtained. Genetic sequence data can also be derived from cell-free DNA present outside of an individual's cellular nuclei. This information can be presented as a dataset including the genetic variation that was identified.

Genetic variation that can be identified in genetic sequence data include copy number variants (or CNVs, differences in the number of repeats of a genetic region), indels (genetic insertions or deletions), single nucleotide variants (or SNVs, nucleotide base differences at a single position), and structural variations (or SVs, including chromosomal rearrangements, translocations, and inversions). Genetic variation (including CNVs, indels, SNVs, and SVs) can be identified by comparing the sequence information obtained from an individual to publicly available human genetic sequence data, as well as de novo approaches that do not rely on publicly available human genetic sequence data.

In particular embodiments, for genetic sequencing, whole blood can be processed to extract DNA. The whole blood can be shipped to a sequencing services lab, where the samples can be processed and analyzed using a sequencing platform. Examples of sequencing services labs include Complete Genomics Inc., the New York Genome Center, or the Illumina FastTrack Sequencing Services. Examples of sequencing platforms include Illumina (e.g. HiSeq), Pacific Biosciences (e.g. Sequel System), Applied Biosystems (e.g. SOLiD sequencing).

In particular embodiments, dynamic analyte data includes measures of one or more of microbiomes 212, one or more metabolomes 214, and/or one or more proteomes 216. The clinical test results 210 can also include dynamic analyte data. In particular embodiments, dynamic analyte data can also include measures of one or more transcriptomes, epigenomes, quantified-self data, combinations thereof, or any dynamic analyte data which can be represented as a quantitative measurement.

Proteome measures can include measurement of at least 5 proteins, at least 10 proteins, at least 50 proteins, at least 100 proteins, or more, in a given sample. In certain embodiments, proteome data can be obtained by measurement of proteins present in patient plasma samples. To prepare plasma samples for proteomic analysis, the most abundant plasma proteins present in the sample can be depleted, using a method such as the Multiple Affinity Removal System (MARS, by Agilent Technologies). Depletion of the most abundant proteins in a sample may aid in the detection of other proteins that are present in the sample at lower abundance.

In particular embodiments, proximity extension assay is a method that can be used to obtain proteomic data. An example of a kit used to perform proximity extension assay is the ProSeek Multiples Immunoassay by Olink. This method involves incubation of samples with DNA-linked antibodies, using pairs of antibodies that bind the same target protein. After antibody incubation, a DNA polymerization reaction is performed. DNA amplification can occur only when each antibody pair binds the target protein because the DNA amplification step requires both DNA molecules to be present. The amplified DNA can be quantified and this information is used to determine the quantity of the protein in the sample.

In particular embodiments, Selected Reaction Monitoring (SRM) is a method that can be used to obtain proteomic data. This method utilizes mass spectrometry to conduct quantitative analysis on a pre-determined set of proteins. Samples can be spiked with known concentrations of heavy-isotope labeled peptides in order to aid in the determination of sample protein concentration.

Microbiome measures can include measurement of at least 5, at least 10, at least 50, at least 100, or more strains or species of microbes, in a given sample. Microbiome analysis can include identification of microbial strains and/or species present in patient samples. Microbiome analysis can also include relative quantification of microbial strains and/or species present in patient samples. Microbiome analysis can be performed to characterize microbial diversity present in the gut or other regions of the body such as skin, mouth, or vagina. Stool samples can be processed to obtain gut microbiome data, and sample processing and analysis can be performed by a sequencing services laboratory, such as Second Genome, DNA Genotek, or uBiome.

To identify distinct prokaryotic strains/species present in a sample, 16S rRNA can be sequenced, and sequences can be used to identify microbes from particular taxonomic groups. The gene that encodes 16S rRNA (a subunit of the prokaryotic ribosome) is commonly used to identify prokaryotic species because the 16S rRNA sequence is usually well-conserved within a given species. Microbiome data can be in the form of 16S Operational Taxonomic Unit (OTU) read counts. OTUs represent distinct taxonomic groupings that can be used to differentiate microbial species, as well as strains. Using read count data, each OTU present in the sample can be analyzed to determine its percent frequency in a given sample, as compared to the other OTUs present in the sample. 16S OTU read count data can be processed to determine alpha-diversity, or the level of microbial diversity within a given sample. 16S OTU read count data can also be processed to determine beta-diversity, or the level of microbial diversity of a given sample as compared to other samples. Microbiome data could also include metagenomic or metatranscriptomic sequencing of the microbiome (full genome or transcriptome sequencing of the microbiome).

Metabolome measures can include measurement of at least 5, at least 50, at least 100, or more metabolites in a given sample. Metabolites include small molecules (typically less than 1500 daltons) that are present in a biological sample. Examples of metabolites include vitamins, fatty acids, and amino acids. For metabolite analysis, patient plasma samples can be analyzed. Plasma samples can be shipped to a metabolomics analysis services center, such as Metabolon. For metabolite analysis, methods of determining metabolite levels include high-performance liquid chromatography, tandem mass spectrometry, colorimetric and/or fluorometric enzymatic assays, and gas chromatography.

Clinical measures can include measurement of at least 1, at least 5, at least 10, at least 20, or more clinical laboratory tests. Clinical laboratory tests include any small metabolite, protein, or other dynamic analyte that has been approved by the United States Federal Drug Administration (FDA) or other United States or international regulatory body for use in diagnosis or monitoring of a biological condition or risk of a biological condition for an individual, such as disease or wellness state. Clinical laboratory tests may be performed in laboratories approved for such functions, such as Quest Diagnostics or LabCorp. Examples of clinical laboratory tests include fasting glucose or glycated hemoglobin (HbA1c) for use in diagnosis and monitoring of type 1 and type 2 diabetes and LDL cholesterol, HDL cholesterol, or blood pressure for use in monitoring the risk of cardiovascular disease or stroke. In some cases, the clinical testing data can include ratios or other combinations or modifications of measures of dynamic analytes.

In particular embodiments, multi-omic longitudinal data refers to at least two of genomic data, dynamic analyte data, and/or clinical test data or environmental test data collected for at least 2, 5, 10, 15, 20, 30, 40, 50, 60, 70 or more points in time. The data collection can be spaced at defined intervals, such as weekly, bi-monthly, monthly, every 2 months, every 3 months, every 6 months, every 9 months, yearly, etc. The data collection need not fall precisely on such representative time points, but may also be within, for example, 1 day, 2 days, 3 days, 4 days, 5 days, 6 days, or 7 days of a target data collection date.

Example environment 200 also includes a computing system 218 configured to receive, store, and analyze the multi-omic data (e.g., clinical or environmental test results 210, microbiomes 212, metabolomes 214, proteomes 216, and/or other dynamic analytes). Although not shown in FIG. 2, computing system 218 may receive data via a network.

In the illustrated example of FIG. 2, the computing system 218 includes one or more processors 220, and memory 222, operably connected to each other such as via a bus 224. Bus 224 may include, for example, one or more of a system bus, a data bus, an address bus, a PCI bus, a Mini-PCI bus, and any variety of local, peripheral, and/or independent buses.

Processor 220 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-chip systems (SOCs), Complex Programmable Logic Devices (CPLDs), etc.

Memory 222 can store data as well as instructions executable by the processor 220. For example, memory 222 can include genome sequence data store 226, clinical or environmental test data store 228, operating system 230, and data correlation identification system 232. Although the illustrative example of FIG. 2 shows the genome sequence data store 226 and the clinical or environmental test data store 228 as part of the computing system 218, in other implementations, the genome sequence data store 226 and/or the clinical or environmental test data store 228 can be accessible to the computing system 218, but located outside of the computing system 218.

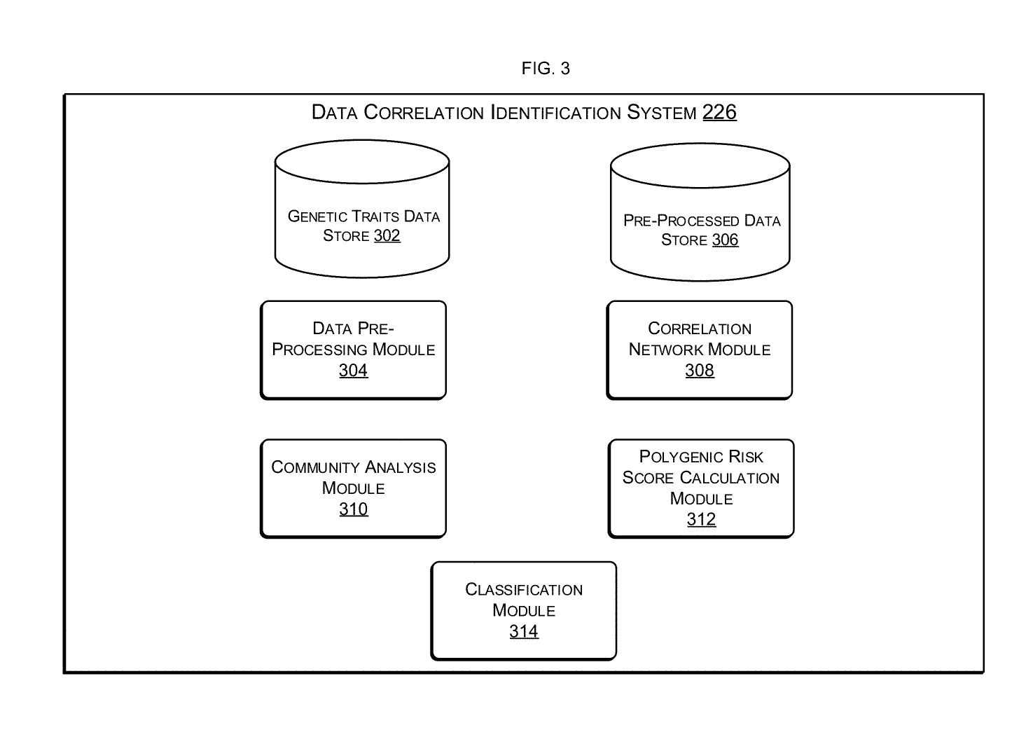

The genome sequence data store 226 can store genome sequence information for a number of individuals 204. The genome sequence data store 226 can also store information indicating correlations between portions of a genome sequence and biological conditions. The clinical or environmental test data store 228 can store information associated with the clinical or environmental test results 210. In addition, the clinical or environmental test data store 228 can store information corresponding to conditions under which the clinical tests and/or environmental tests were performed. The operating system 230 can include computer-readable instructions that are executable by the processor 220 to manage software and hardware resources of the computing system 218. The operating system 230 can include a Linux operating system, a Windows operating system, an Apple operating system, or another type of operating system. The data correlation identification system 232 can include computer-readable instructions that are executable by the processor 220 to perform operations that correlate biological conditions to biological indicators, such as dynamic analytes. The operations of the data correlation system 232 will be described in more detail with respect to FIG. 3.

Memory 222 is an example of computer-readable media. As described above, memory 222 can store instructions executable by processor 220. Computer-readable media (e.g., memory 222) can also store instructions executable by external processing units such as by an external CPU, an external GPU, and/or executable by an external accelerator, such as an FPGA type accelerator, a DSP type accelerator, or any other internal or external accelerator. In various examples, at least one CPU, GPU, and/or accelerator is incorporated in computing system 218, while in some examples one or more of a CPU, GPU, and/or accelerator is external to computing system 218.

Computer-readable media may include computer storage media and/or communication media. Computer storage media can include volatile memory, nonvolatile memory, and/or other persistent and/or auxiliary computer storage media, removable and non-removable computer storage media implemented in any method or technology for storage of information such as computer-readable instructions, data structures, program modules, or other data. Memory 222 can be an example of computer storage media. Thus, the memory 222 includes tangible and/or physical forms of media included in a device and/or hardware component that is part of a device or external to a device, including random-access memory (RAM), static random-access memory (SRAM), dynamic random-access memory (DRAM), phase change memory (PRAM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer storage media, communication media may embody computer-readable instructions, data structures, program modules, or other data in a modulated data signal, such as a carrier wave, or other transmission mechanism. As defined herein, computer storage media does not include communication media. That is, computer storage media does not include communication media consisting solely of a modulated data signal, a carrier wave, or a propagated signal, per se.

Computing system 218 can belong to, or include, a variety of categories or classes of devices such as traditional server-type devices, desktop computer-type devices, mobile-type devices, special purpose-type devices, embedded-type devices, and/or wearable-type devices. Thus, although illustrated as a single type of device, computing system 218 can include a diverse variety of device types and are not limited to a particular type of device.

FIG. 3 illustrates select components of an example data correlation identification system 226 as described herein. Example data correlation identification system 226 includes genetic traits data store 302, data pre-processing module 304, pre-processed data store 306, correlation network module 308, community analysis module 310, and polygenic risk score calculation module 312.

Genetic traits data store 302 includes data identifying genetic traits, which can be used to identify genetic traits of a particular individual 104 based on a portion of the genome sequence of the particular individual 104 or the whole genome sequence of the particular individual 104. The genetic traits data store 302 can include at least a portion of the genomic data 208 of FIG. 2. In some cases, the genetic traits data store 302 can also be populated from The National Human Genome Research Institute's GWAS catalog, which presently lists results from more than 2000 published studies. In particular embodiments, these studies link genetic sequences or groupings of genetic sequences with increased or decreased risk for phenotypic outcomes in individuals (e.g., BRCA1 and BRCA2 sequences linked with increased or standard risk for breast cancer).

In an example implementation, in order to increase the probability of identifying a statistically significant correlation that passes multiple hypothesis correction, a strict filtering procedure may be applied to the GWAS catalog data. For example, analyses may exclude: genetic traits with conflicting effects in the literature; genetic traits that lack a particular degree of statistical significance; phenotypic linkages based on small SNV number; and/or genetic traits based on a sample size that is smaller than a predefined number. In this way, the efficiency and accuracy of the data correlation identification system 226 is improved with respect to other systems that do not implement filtering procedures for the information included in the GWAS catalog because less information is analyzed to determine relationships between biological conditions and dynamic analytes. In particular embodiments the improvement is reflected in reduced processing times.

In particular embodiments, SNVs with a genome-wide significant p-value <5×10e-8 can be included. In particular embodiments, SNVs with a p-value <10e-6 can be included. In particular embodiments, SNVs with a p-value <10e-4 can be included. In particular embodiments, SNVs with a p-value <10e-2 can be included. In particular embodiments, SNVs with a p-value that meets a significant threshold sufficient to distinguish them from random noise can be included.

Studies which contain few (e.g., 5) SNVs are likely to produce a vector of cumulative genetic variation with low entropy, where almost all values are identical save a few. Depending on the number of individuals 204 for whom genomic data is available, such low entropy measurements may be more likely to produce spurious correlations. Therefore, in an example implementation, traits associated with seven, six, five, four, three or fewer SNVs can be excluded.

In particular embodiments, filtering may be performed based on sample size. In an example, studies having a sample size less of less than 5000, 4000, 3000, 2000, or 1000 individuals can be excluded.

In particular embodiments, filtering may be performed to exclude studies that examined the same trait as examined in another study. For example, in the event that multiple studies examined the same trait, the study with the largest sample size may be kept and the others excluded.

Data pre-processing module 304 can pre-process the results from tests performed by a laboratory, such as the laboratory 206 of FIG. 2, and transform each dataset into comparable data vectors for statistical analysis. In example implementations, data pre-processing module 304 can receive measurements representing the genomic data 208, the clinical test results 210, the microbiomes 212, the metabolomes 214, and the proteomes 216. The genomic data 208 can be compared against information stored in the genetic traits data store 302 to identify genetic traits represented within the genomic data 108 of individuals 104 to generate various genetic risk scores.

The other measurements may be mean centered and scaled by the standard deviations of the observed measurements. In some examples, if multiple values are available for a particular individual 204 (i.e., lab results from multiple samples collected on different days over a period of time), a mean dynamic analyte value can be calculated for use by the correlation network module 308. Additionally, if multiple values are available for a particular individual 204, a standard deviation dynamic analyte value can be calculated for use by the correlation network module 308. Further, if multiple values are available for a particular individual 204, the difference between two time points can be calculated for use by the correlation network module 308. Thus, difference measurements can be utilized by the correlation network module 308. Also, microbiome measurements 212 may be compared independently at the domain, phylum, class, order, family, genus, species, or OTU taxonomic levels.

Data resulting from data pre-processing module 304 may be stored in pre-processed data store 306, which may be accessed by the correlation network module 308. Correlation network module 308 can be configured to generate a correlation network based on the various data sources (e.g., genetic traits identified in the genomic data 208, clinical test results 210, microbiomes 212, metabolomes 214, and proteomes 216). In some examples, the correlation network module 306 can generate a correlation network for each pair of data sources. In example implementations, correlation network module 206 can build an age- and sex-adjusted correlation network based on Spearman correlations. In this correlation network, vertices (V) correspond to dynamic analytes, and an edge (E) exists between two vertices if and only if a significant (p_{adj}<0.05) correlation was observed after correction for multiple hypotheses using the method of Benjamini & Hochberg (Benjamini & Hochberg (1995). Journal of the Royal Statistical Society: Series B (Statistical Methodology) 57, 289-300.). In particular examples, missing data may be excluded; that is, individuals for whom values are missing may be dropped from pairwise comparisons utilizing that value.

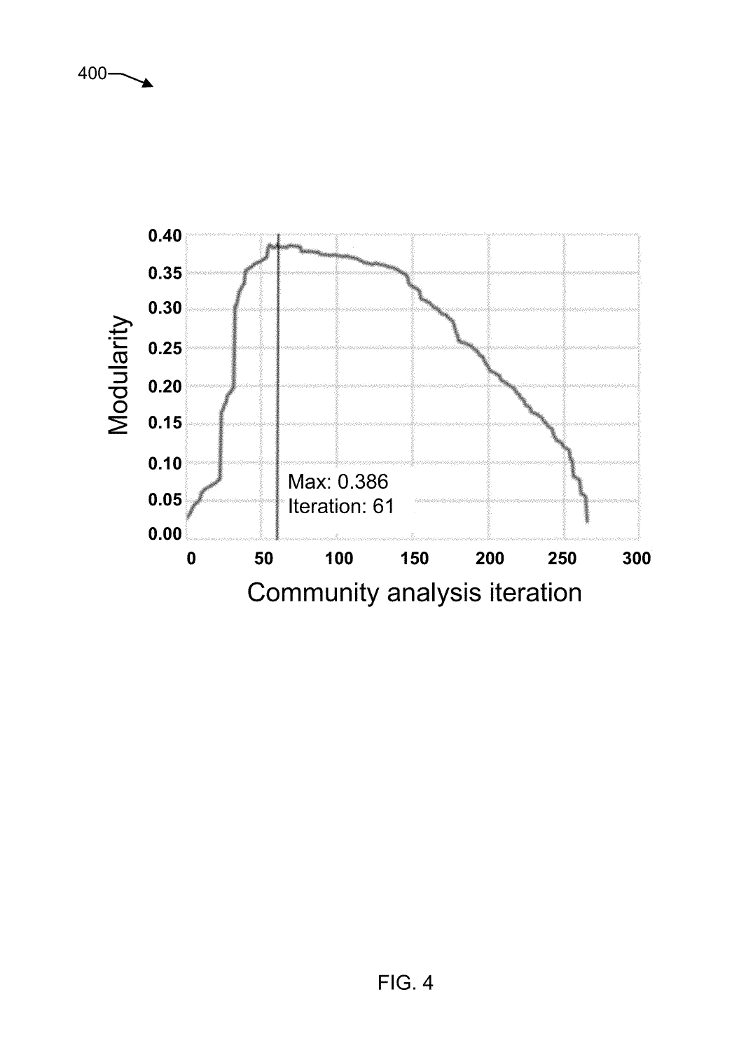

Community analysis module 310 is configured to identify clusters of interrelated measurements across individuals. The clusters of interrelated measurements can be depicted by a cross-correlation network generated by the correlation network module 308. That is, one or more cross-correlation networks generated by the correlation network module 308 can be utilized as an input to the community analysis module 310. Community analysis module 310 may be configured to use the method of Girvan and Newman. This method involves iteratively calculating edge betweenness centrality on a network: the number of weighted shortest paths from all vertices to all other vertices that pass over that edge. After each iteration, the edge(s) with the highest betweenness centrality are removed and the process is repeated until only individual nodes remain (Girvan & Newman (2002). Proc. Natl. Acad. Sci. U.S.a. 99, 7821-7826.). The maximization of modularity may be used to identify communities at a particular hierarchical level of the dendrogram (Newman, (2006). PNAS 103, 8577-8582). In some cases, the communities identified at the maximization of modularity can be used to determine correlations between biological conditions and dynamic analytes. In particular implementations, after a first set of one or more iterations, a first group of nodes may be connected by one or more edges, while some nodes that were previously connected before the first set of one or more iterations are no longer connected by an edge. After a second set of one or more iterations, a second group of nodes may be connected by one or more edges. The second group of nodes can have fewer nodes than the first group of nodes. The first group of nodes can indicate correlations between a first set of dynamic analytes and a first set of biological conditions and the second group of nodes can indicate correlations between a second set of dynamic analytes and a second set of biological conditions. As iterations of the community analysis method are performed, the presence of correlations between certain dynamic analytes and biological conditions can be identified with greater accuracy. FIG. 4 shows the number of iterations in an inter-omic community analysis versus modularity with the maximum modularity being 0.386 at iteration 61 of community pruning.

The community analysis module 308 can produce different communities that are included in the cross-correlation network. The communities can represent a cluster of physiologically-related dynamic analytes and/or biological conditions. Each community can include a number of vertices and a number of edges. In some cases, a community can have at least 3 vertices, at least 5 vertices, at least 10 vertices, at least 25 vertices, at least 50 vertices, at least 100 vertices, at least 150 vertices, at least 200 vertices, at least 250 vertices, or at least 300 vertices. In illustrative examples, communities produced by the community analysis module 308 can have from 3 vertices to 1,000 vertices. Additionally, a community can have at least 3 edges, at least 10 edges, at least 25 edges, at least 50 edges, at least 100 edges, at least 250 edges, at least 500 edges, at least 750 edges, at least 1000 edges, at least 1250 edges, at least 1500 edges, at least 1750 edges, or at least 2000 edges. In illustrative examples, communities produced by the community analysis module 308 can have from 3 vertices to 10,000 vertices. In particular embodiments, communities produced by the community analysis module 308 can have a p_{adj} value of less than 0.05. Additionally, sub-communities can be identified by the community analysis module 308 within a particular community.

In some particular examples, the community analysis module 308 can identify a community related to cardiometabolic health with a sub-community related to total cholesterol and LDL cholesterol. In another particular example, the community analysis module 308 can identify a community corresponding to plasma serotonin. In an additional example, the community analysis module 308 can identify a community corresponding to microbiome α-diversity.

Polygenic risk score calculation module 312 is configured to calculate each individual's genetic risk score for specific conditions and phenotypic traits, and to correlate those scores with a trait of interest. In example implementations, the variants with genome-wide significance that are associated with a trait of interest are identified from the NHGRI GWAS catalog, along with their pre-calculated effect sizes and effect alleles. The effect alleles can include SNVs that may indicate a biological condition. The effect size can correspond to a contribution of an SNV to genetic variations of a trait. In particular implementations, the effect size can be related to a coefficient of a linear model that can indicate one or more outcomes. For each variant, the number of effect alleles within an individual are multiplied by the effect size. The resulting scores are summed over all variants with genome-wide significance that are associated with the trait of interest, resulting in a polygenic score for that trait for that individual. In some embodiments, variants with genome-wide significance are derived from a single published study. In particular embodiments, variants with genome-wide significance are derived from multiple independent studies investigating the same trait. In additional embodiments, a subset of variants from a single published study are used.

Classification module 314 is configured to take a set of inputs from pre-processed data store 306 for a particular individual as well as a biological condition, and indicate whether or not the individual has the condition or not. In some embodiments, classification module 314 can return a probability associated with the classification.

In example implementations, for each pairwise set of data (e.g. clinical tests vs. proteomics, clinical tests vs. metabolomics, polygenic scores vs proteomics, etc.), each measurement from the first dataset is correlated with every measurement from the second dataset using Spearman's ρ, which returns a coefficient between −1 and +1, where −1 is perfect negative correlation and +1 is perfect positive correlation between two ranked variables. Spearman's p is a non-parametric test that works on the ranks and is therefore robust to outliers as well as insensitive to the distributions of the variables being compared. Once the coefficients and p-values are computed, all p-values are adjusted for multiple hypothesis testing using the method of Benjamini and Hochberg (Benjamini and Hochberg, 1995). An adjusted p-value cutoff value may be selected as a significance level. In illustrative examples, an adjusted p-value cutoff of 0.05 is used as the significance level. The classification module 314 can utilize the results of these calculations to determine a probability for a classification of an individual with a particular biological condition. In some examples, only inter-omic correlations are used for community and classification analysis. However, it is contemplated that there may be benefits of also analyzing intra-omic (e.g. metabolomics vs. metabolomics) correlations.

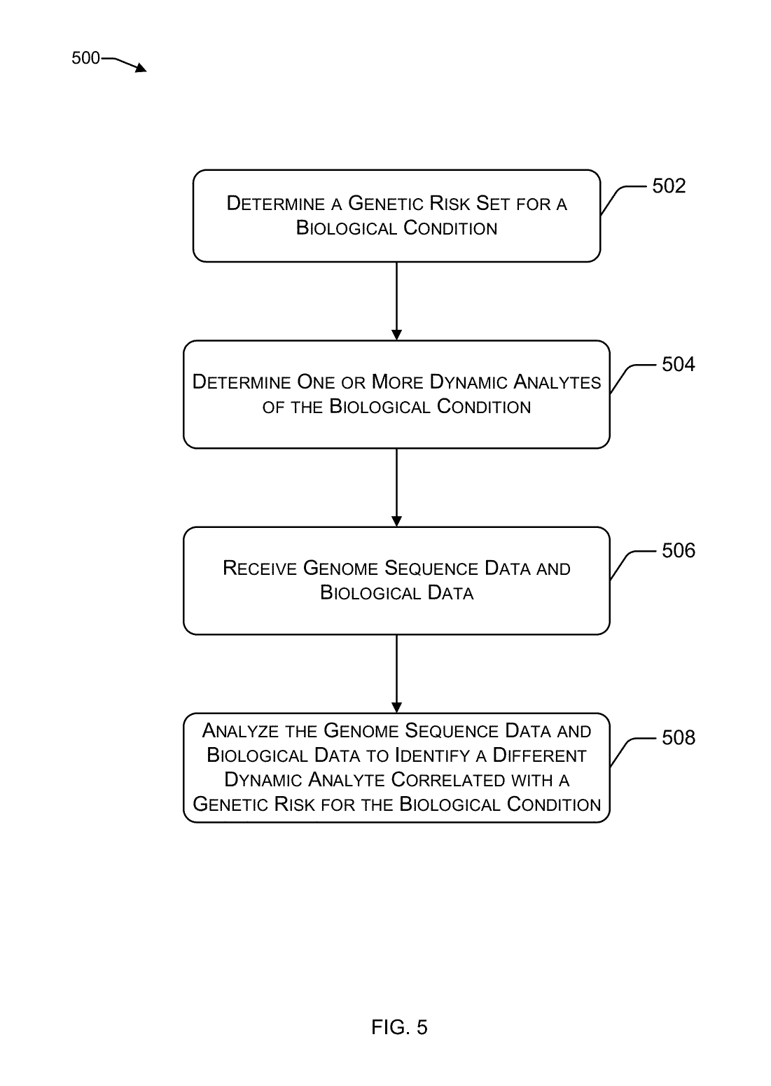

FIGS. 5-9 illustrate example methods for performing multi-omic longitudinal data analysis. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

FIG. 5 illustrates an example method 500 for identifying dynamic analytes indicative of condition onset that are independent of biochemical products of a known genetic risk set. At block 502, a genetic risk set for a biological condition is determined. For example, genetic traits data store 302 identifies genetic risk for each of any number of biological conditions as identified in any number of genome-wide association studies.

At block 504, one or more dynamic analytes of the biological condition are determined. In some cases, the dynamic analytes can be determined based at least partly on information from previously performed studies that correlate certain dynamic analytes with particular biological conditions.

At block 506, genome sequence data and biological data is received. For example, blood samples obtained from any number of individuals are processed by a laboratory to generate genome sequence data associated with each of the individuals. Furthermore, other biological samples, such as blood, urine, stool, and saliva samples, may be obtained from the individuals and processed by a laboratory to generate the biochemical data, which may include, for example, clinical test results 210, microbiome measurements, metabolome measurements, and proteome measurements.

At block 508, the genome sequence data and biological data is analyzed to identify a different dynamic analyte that is correlated with a genetic risk for the biological condition. For example, correlation network module 308 can generate one or more correlation networks between the genome sequence data and the biological data. Community analysis module 310 can then identify clusters of interrelated measurements across individuals to identify one or more dynamic analytes that are correlated with a genetic risk for the condition. A dynamic analyte identified for the biological condition can be previously unknown with regard to the biological condition. That is, the dynamic analyte may not be included in pre-existing literature indicating a relationship between the dynamic analyte and the biological condition.

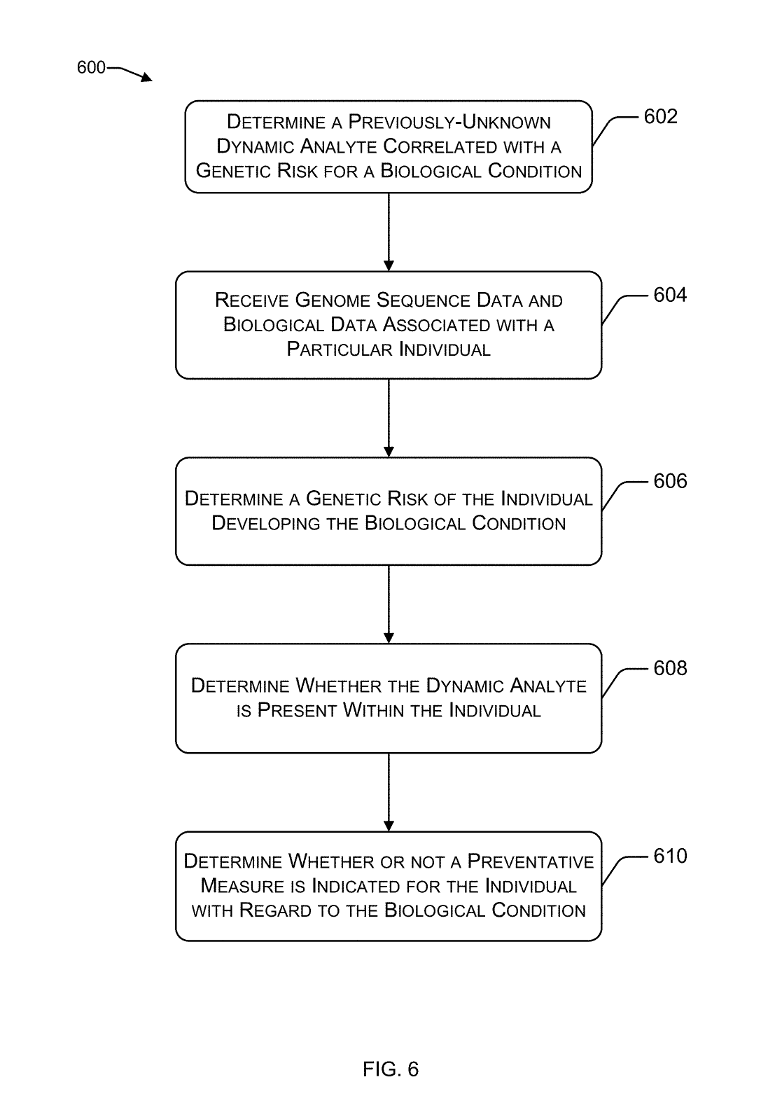

FIG. 6 illustrates an example method 600 for identifying subjects appropriate for a preventative measure based on genetic risk and expression of an independent dynamic analyte. At block 602, a previously-unknown correlation between a biological indicator and a genetic risk for a biological condition is determined. For example, the process described above with reference to FIG. 5 is performed to identify a correlation between the biological indicator and a genetic risk for the condition.

At block 604, genome sequence data and biological data associated with a particular individual is received. For example, a blood sample obtained from the particular individual is processed by a laboratory to generate genome sequence data associated with the particular individual. Furthermore, other biological samples, such as blood, urine, stool, and saliva samples, may be obtained from the individual and processed by a laboratory to generate the biological data, which may include, but is not limited to, clinical test results 210, microbiome measurements, metabolome measurements, and proteome measurements.

At block 606, a genetic risk of the individual developing the biological condition is determined. For example, polygenic risk score calculation module 312 can calculate the individual's genetic risk of developing the biological condition based, at least in part, on a comparison of the individual's genome sequence data and data in the genetic traits data store 302.

At block 608, it is determined whether or not the dynamic analyte is present within the individual. For example, data correlation identification system 226 examines the biological data stored in the pre-processed data store to determine whether or not the dynamic analyte is present within the individual. In certain implementations, the individual can be included in a group of individuals that are monitored over a period of time. For example, amounts of dynamic analytes present in the individual can be monitored. In some examples, the amount of one or more dynamic analytes present in the individual can change and indicate that the individual is developing at least one phenotype for a biological condition (i.e., the biological condition is emerging in the individual).

The presence of a dynamic analyte or an amount of a dynamic analyte present in a subject can be determined utilizing a number of techniques. In some cases, the presence of amount of a dynamic analyte present in a subject can be determined based on a type of the dynamic analytes. For example, proteins can be detected according to a first set of techniques, while metabolomics can be detected according to another set of techniques, and microbiome data can be detected according to still another set of techniques. In particular implementations, dynamic analytes can be detected using techniques where the dynamic analytes are contacted with a substance and the dynamic analytes bind to that substance or to a binding agent coupled to the substance. To illustrate, proteins can be detected by a portion of the protein binding to DNA, RNA, an amino acid sequence (e.g., a peptide), another protein (e.g., an enzyme or an antibody), or another molecule that interacts with the protein. In a particular example, proteins can be detected using protein panels produced by Olink Proteomics (Uppsale, Sweden). Dynamic analytes can also be identified through sequencing techniques, such as DNA or RNA sequencing. RNA sequencing can refer to determining an RNA sequence by reverse-transcribing the RNA to complementary DNA (cDNA), and sequencing the resulting cDNA. RNA sequences can also be determined by sequencing the exons of the genomic DNA encoding the RNA. Sequencing techniques include, for example, chain termination sequencing, sequencing-by-synthesis, and pyrosequencing. Other techniques can be used to detect dynamic analytes through the binding of the analytes to one or more surfaces. In some scenarios, dynamic analytes and/or amounts of dynamic analytes can be identified through chromatographic techniques. In various embodiments, assays can be used to detect the presence of and/or amounts of dynamic analytes. The assays can utilize binding techniques to detect the presence or amount of a dynamic analyte, such as a ligand-binding assay or an immunoassay that binds an antibody or antigen related to the dynamic analyte.

At block 610, it is determined whether or not a preventative measure or treatment is indicated for the individual with regard to the biological condition. For example, data correlation identification system 226 can determine whether or not the particular individual's genetic risk of developing the biological condition is greater than a threshold genetic risk. If the particular individual's genetic risk of developing the biological condition is greater than the threshold risk, then if it was determined that the biochemical characteristic is present within the individual, the data correlation identification system 226 determines that a preventative measure is indicated for the individual with regard to the biological condition. The preventative measure or treatment can be initiated to modify the amount of the dynamic analyte present in the individual to prevent development of the biological condition or reverse development of the biological condition within the individual. Wellness of a population can be preserved when treatment of a biological condition is provided when the biological condition begins to emerge in individuals.

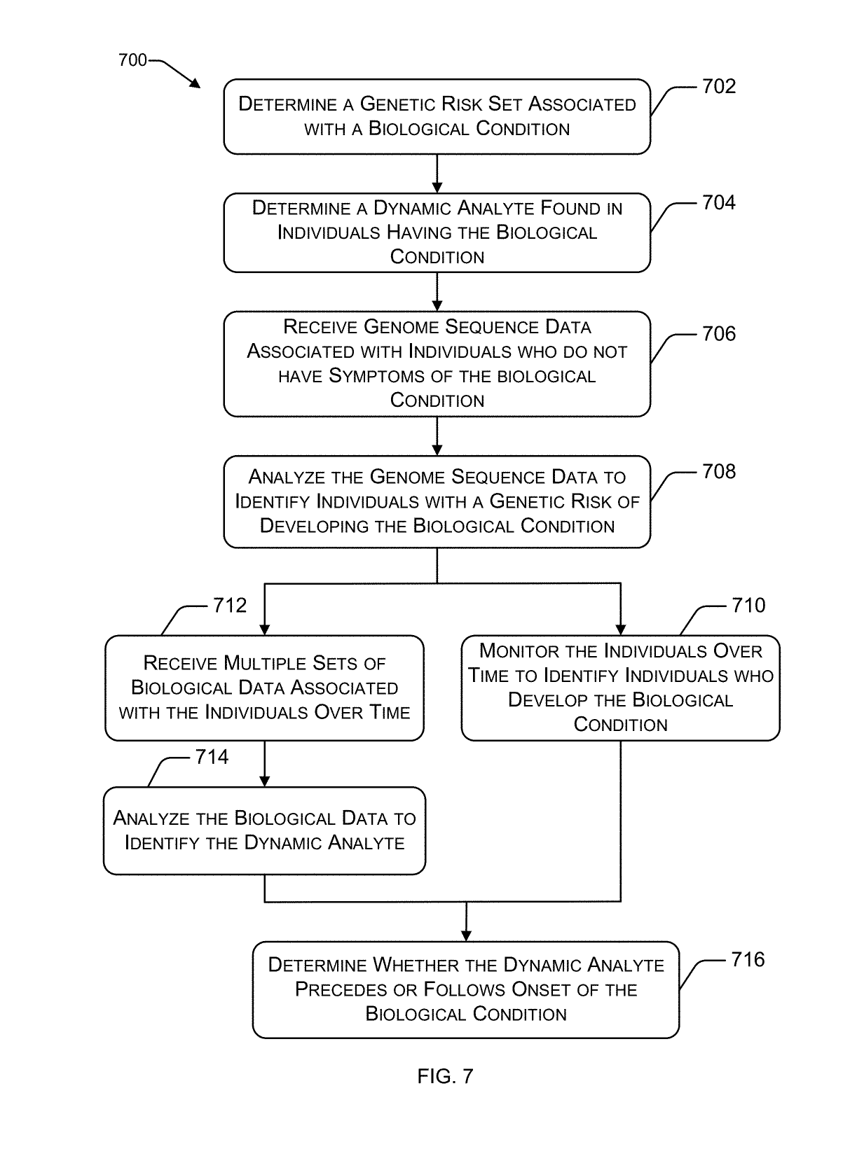

FIG. 7 illustrates an example method 700 for determining whether a dynamic analyte precedes or follows onset of a biological condition. At block 702, a genetic risk set associated with a biological condition is determined. For example, genetic traits data store 302 can include information identifying genetic risk for each of any number of biological conditions as identified in any number of genome-wide association studies.

At block 704, a dynamic analyte found in individuals having a biological condition is determined. In some cases, the dynamic analyte can be found in elevated amounts (i.e., amounts higher than individuals that do not exhibit one or more phenotypes for the biological condition) when a biological condition is present in the individuals. In other situations, the dynamic analyte can be found in reduced amounts (i.e., amounts lower than individuals that do not exhibit one or more phenotypes for the biological condition) when a biological condition is present in the individuals.

At block 706, genome sequence data is received in association with individuals who do not have symptoms of the biological condition. For example, blood samples obtained from any number of individuals is processed by a laboratory to generate genome sequence data associated with each of the individuals.

At block 708, the genome sequence data is analyzed to identify individuals with a genetic risk of developing the biological condition. For example, polygenic risk score calculation module 312 can compare the genome sequence data generated at block 706 with the data stored in genetic traits data store 302 to identify individuals having a genetic risk of developing the biological condition.

At block 710, the individuals are monitored over time to identify individuals who develop the biological condition. For example, received medical records or self-reporting by the individuals may be used to determine when particular individuals experience onset of the biological condition.

At block 712, over a period of time, multiple sets of biological data associated with the individuals are received. For example, biological samples, such as blood, urine, stool, and saliva samples, may be obtained from the individuals and processed by a laboratory to generate the biological data, which may include, but is not limited to, clinical test results 210, microbiome measurements, metabolome measurements, and proteome measurements.

At block 714, the biological data is analyzed to identify the dynamic analytes. For example, classification module 314 can analyze the biological data generated according to block 712 to determine, at different points in time, which individuals indicated a presence of the dynamic analyte.

At block 716, it is determined whether or not the dynamic analytes precedes or follows condition onset. For example, classification module 314 can compare dates at which the dynamic analyte was present with biological condition onset dates for the individuals who initially showed no symptoms of the biological condition, but later developed the biological condition. If a threshold percentage of individuals presented with the dynamic analytes prior to condition onset, it is determined that the dynamic analyte precedes biological condition onset. In some cases, the classification module 314 can determine that a dynamic analyte that was previously identified as indicating onset of the biological condition actually precedes biological condition onset. Additionally, the classification module 314 can analyze the biological data to determine whether a dynamic analyte increases or decreases before or after condition onset

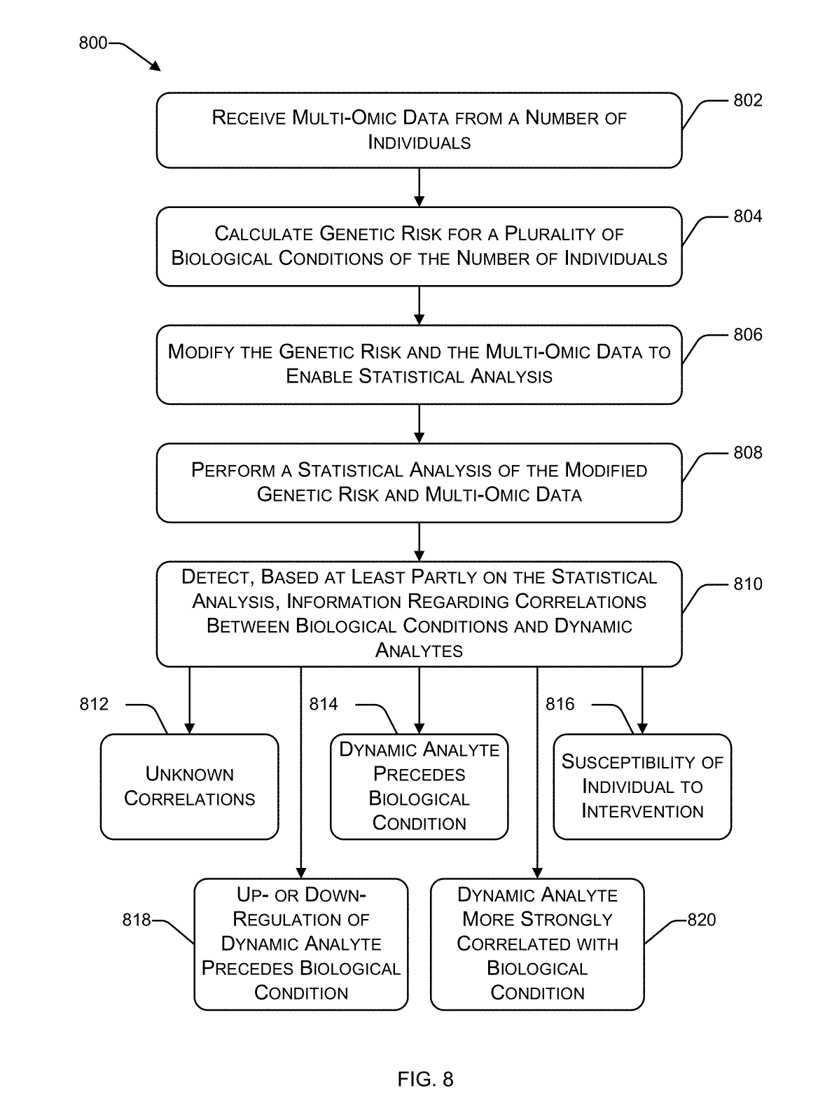

FIG. 8 is a flow diagram of an example method 800 to analyze multi-omic data and genetic risk scores to identify information about relationships between biological conditions and dynamic analytes. At 802, the method 800 includes measuring and/or receiving multi-omic data from a number of individuals. The multi-omic data can include genomic data, such as genome sequence data sets, and one or more dynamic analyte data sets, such as at least one of proteomic data, metabolomic data, microbiome data, transcriptomics data, epigenomic data, or clinical test results data. In some examples, the multi-omic data can be obtained with respect to at least 100 individuals, at least 250 individuals, at least 500 individuals, at least 1000 individuals, at least 1500 individuals, at least 2000 individuals, at least 2500 individuals, at least 5000 individuals, or at least 10,000 individuals. In particular implementations, the genomic data can include whole genome sequence data. Additionally, the microbiome data can include 16s rRNA sequencing data. In various implementations, the microbiome data can include full metagenomics sequencing or metatranscriptomics sequencing. In certain implementations, a subset of the individuals can be associated with genetic risk for a biological condition with a remaining subset of the individuals not being associated with genetic risk for the biological condition. Further, the proteomics data can include plasma depleted of the 14 most abundant plasma proteins per sample.

At 804, the method 800 includes calculating genetic risk for a plurality of biological conditions for the number of individuals. The genetic risk can be calculated utilizing the genomic data and genome-wide association studies (GWAS). In particular implementations, calculating the genetic risk can include for each variant of one or more variants associated with a genetic trait, multiplying a number of effect alleles by an effect size to determine a respective score for each variant in order to produce a plurality of scores for the genetic trait. The plurality of scores for the genetic trait can then be added to produce a sum that produces a polygenic score for each individual with respect to the genetic trait. The score can indicate a probability that an individual will develop a phenotype with respect to the genetic trait. In some examples, the one or more variants can be derived from a single published study. In other examples, the one or more variants can be derived from multiple studies investigating the genetic trait. In additional examples, the one or more variants can include a subset of variants from a single published study.

In various implementations, at least one GWAS study can be excluded from calculating the genetic risk for the number of individuals. The at least one GWAS study can be excluded from calculating the genetic risk for the number of individuals based at least partly on a sample size of the at least one GWAS study being less than 5000 individuals. The at least one GWAS study can also be excluded from calculating the genetic risk for the number of individuals based at least partly on a description of the genetic risk being associated with less than 5 single nucleotide variants (SNVs). In some cases, at least one GWAS study can be excluded from calculating the genetic risk for the number of individuals based at least partly on the at least one GWAS study lacking at least one SNV with a p-value of <10-e8.

At 806, the method 800 includes modifying the genetic risk and the multi-omic data to enable statistical analysis of the genetic risk and the multi-omic data. Modifying the genetic risk and multi-omic data can include normalizing and transforming the genetic risk and the multi-omic data into comparable data vectors. In implementations where the multi-omic data includes metabolomics data, normalizing the metabolomic data can include scaling across samples included in the metabolomic data. Further, in implementations where the multi-omic data includes proteomics data, normalizing the genetic risk and multi-omic data can include normalizing extension positive control and negative control C_{q} values. In some cases, transforming the genetic risk and multi-omic data into comparable data vectors can include performing at least one of mean-centering or standard deviation scaling of the genetic risk and multi-omic data. In additional examples, transforming the genetic risk and multi-omic data into comparable data vectors can include at least one of log transformation, exponentiation, or determining differences between values of at least one dynamic analytes at multiple points in time. Transforming the genetic risk and multi-omic data can also include combining multiple dynamic analytes into a single analyte through a linear or nonlinear combination.

At 808, the method 800 includes performing a statistical analysis of the modified genetic risk and multi-omic data. The statistical analysis can include performing linear regression with respect to the modified genetic risk and multi-omic data. In other implementations, the statistical analysis can include performing nonlinear regression with respect to the modified genetic risk and multi-omic data. In particular implementations, the statistical analysis can be performed independently at the phylum, class, order, family, genus, species, and operational taxonomic unit (OUT) taxonomic levels. The statistical analysis can include calculating Spearman's ρ following data partitioning by dynamic analyte type.

At 810, the method 800 can include detecting, based at least partly on the statistical analysis, information regarding correlations between biological conditions and dynamic analytes. In some cases, operation 810 can detect unknown correlations 812 between dynamic analytes and biological conditions. In particular implementations, the unknown correlations 812 can include a dynamic analyte being correlated with a biological condition where the dynamic analyte was not previously known to correlate with genetic risk for the biological condition at the time of the statistical analysis. Additionally, the unknown correlations 812 can include a dynamic analyte that was not previously known to correlate with the biological condition in GWAS at the time of the statistical analysis.

Operation 810 can also detect that a dynamic analyte precedes a biological condition 814. To illustrate, a dynamic analyte can be correlated with genetic risk for a biological condition where the dynamic analyte is indicative of the genetic risk of developing the biological condition before the biological condition emerges in an individual. Additionally, operation 810 can detect susceptibility of an individual to intervention 816 for one or more biological conditions. The susceptibility of an individual to intervention 816 can be based at least partly on genetic risk of the individual for a given biological condition and an amount of a dynamic analyte associated with genetic risk for the biological condition. The susceptibility of an individual to intervention 816 can also be based at least partly on an amount of the dynamic analyte present in an individual. Further, the susceptibility of the individual to intervention 816 can be based at least partly on whether an amount of the dynamic analyte present in the individual is commensurate or not commensurate with genetic risk of the individual for developing the biological condition. In situations where the amount of the dynamic analyte present in the individual is not commensurate with the genetic risk of an individual to develop the biological condition, a behavioral intervention or an environmental intervention to change the amount of the dynamic analyte present in the individual in a way to decrease the risk of an adverse outcome for the individual with respect to the biological condition. For example, if the amount of the dynamic analyte present in the individual indicates that an adverse outcome with respect to a biological condition is more likely for an individual based on the genetic risk of the individual associated with the biological condition, then an intervention can be provided that can modify the amount of the dynamic analyte present in the individual to decrease the risk of an adverse outcome for the individual with respect to the biological condition.

Further, operation 810 can detect that up- or down-regulation of a dynamic analyte precedes a biological condition 818. In some cases, the up- or down-regulation of the dynamic analyte was only previously known to be associated with one or more phenotypes of the biological condition and not previously known to take place before the biological conditions has developed in an individual. Additionally, operation 810 can detect that a dynamic analyte is more strongly correlated with a biological condition 820 than previously identified dynamic analytes or clinical test results correlated with the biological condition. Detecting that a dynamic analyte is more strongly correlated with a biological condition 820 can also include detecting that the dynamic analyte is more strongly correlated with genetic risk for the biological condition than previously identified dynamic analytes that are also correlated with genetic risk for the biological condition.

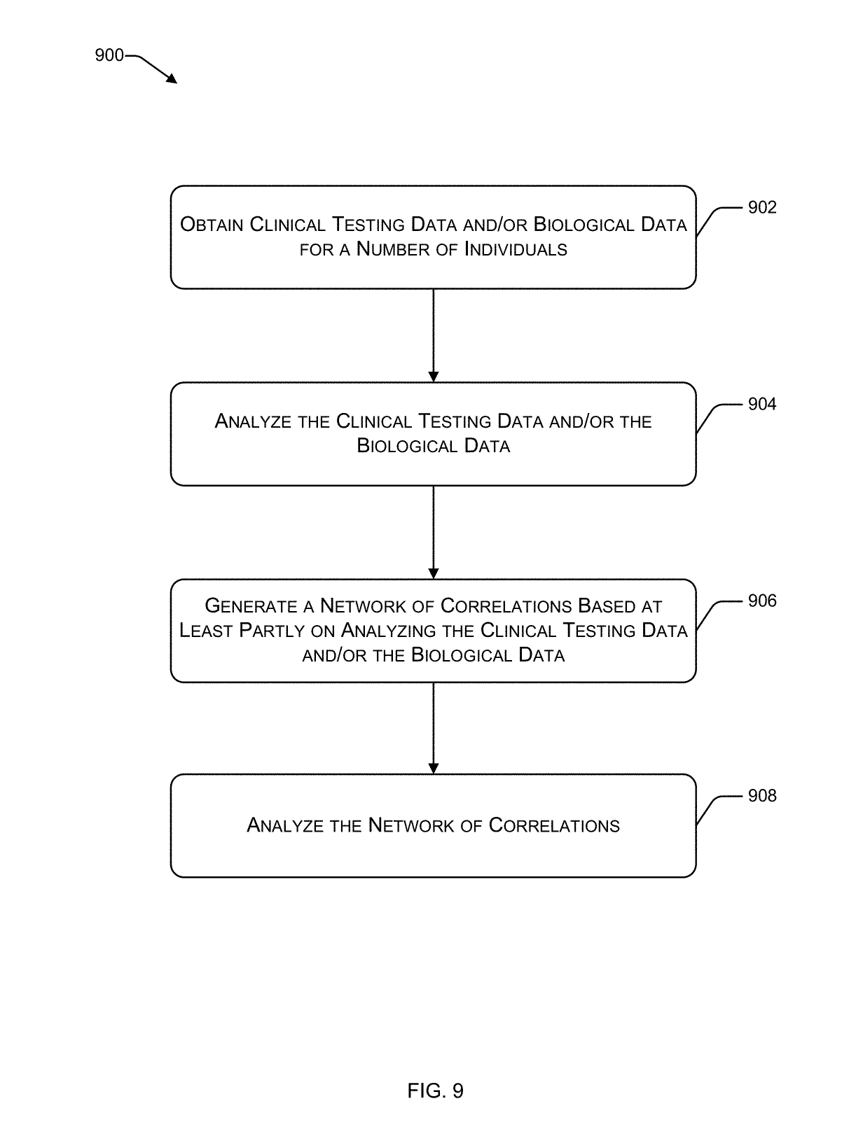

FIG. 9 is a flow diagram of an example method 900 to produce a network of correlations based on biological data and/or clinical testing data and analyzing the network of correlations to identify information about relationships between biological conditions and dynamic analytes. At 902, the method 900 includes obtaining clinical testing data for a plurality of clinical tests and/or biological data for a number of individuals. The clinical testing data can be obtained from various clinical tests that indicate the presence and/or concentrations of dynamic analytes. The biological data can indicate amounts of dynamic analytes for the individuals. In certain implementations, at least a portion of the individuals for which the biological data is obtained can be asymptomatic with respect to one or more biological conditions. An individual can be asymptomatic with respect to a biological condition when that individual is not expressing phenotypes corresponding to the biological condition.

At 904, the method 900 includes analyzing the clinical testing data and/or the biological data. Analyzing the clinical testing data and/or the biological data can include determining respective correlations between at least one of pairs of clinical tests, pairs of dynamic analytes, or pairs including a clinical test and a dynamic analyte.

At 906, the method 900 includes generating a network of correlations that includes at least a portion of the respective correlations. The network of correlations includes vertices and edges between respective pairs of the vertices. Each vertex of the network of correlations corresponds to a clinical test or a dynamic analyte. The edges of the network of correlations indicate a correlation between a pair of clinical tests, a pair of dynamic analytes, or a correlation between a clinical test and a dynamic analyte. In certain implementations, a correlation between nodes connected by individual edges in the network of correlations can be associated with a value that is above a threshold value.

At 908, the method 900 includes analyzing the network of correlations. Analyzing the network of correlations can include determining a number of pre-existing dynamic analytes for a biological condition based at least partly on biological data obtained from individuals that exhibit phenotypes of the biological condition and, also, determining one or more additional dynamic analytes for the biological condition based at least partly on the network of correlations. The pre-existing dynamic analytes can be correlated with the biological condition in previously published literature. The additional dynamic analytes may not be associated with the biological condition in previously published literature. In some cases, at least one additional dynamic analyte can be the subject of a new clinical trial. The new clinical trial can investigate an intervention for the biological condition based at least partly on one or more additional dynamic analytes studied in the new clinical trial. In certain implementations, the intervention can be provided to individuals exhibiting one or more phenotypes of the biological condition.

Analyzing the network of correlations can also include determining that individuals are asymptomatic with respect to one or more biological conditions and performing an analysis of the biological data of the individuals utilizing the network of correlations. In some implementations, the analysis of the biological data of the individuals utilizing the network of correlations can determine a probability that individuals will exhibit one or more phenotypes of the biological condition. In particular implementations, analyzing the network of correlations can include determining that the probability an individual will exhibit one or more phenotypes of a biological condition is greater than a threshold probability and identifying an intervention for the individual. The intervention can be designed to reduce the probability that the individual will exhibit the one or more phenotypes of the biological condition.

In various implementations, analyzing the network of correlations to identify certain groups of vertices and edges. The groups can sometimes be referred to as “communities.” The groups can indicate a number of dynamic analytes that correlate with a biological condition. In some cases, the dynamic analytes correlated with a biological condition can be previously unknown to have been associated with the biological condition. Additionally, the dynamic analytes correlated with a biological condition may have previously been associated with onset of the condition, whereas the network of correlations can be utilized to determine one or more of the dynamic analytes can be present before individuals exhibit phenotypes of the biological condition.

The edges between vertices of the network of correlations can be determined based at least partly on a measure of the correlation between the vertices. The measure of the correlation can be a coefficient, such as Spearman's ρ, that has a value between −1 and +1, where +1 indicates a perfect positive correlation and −1 indicates a perfect negative correlation. Spearman's ρ can provide more accurate results for the correlations included in the network of correlations than other statistical coefficients. For example, Spearman's ρ can be utilized instead of Pearson's r because Pearson's r can be biased by outliers. Additionally, Spearman's ρ can be utilized instead of parametric tests, such as Pearson's r, because parametric tests can be sensitive to the distribution of the variables being compared whereas non-parametric tests are robust to outliers and insensitive to the distribution of the variables being compared. Pairs of data (e.g., different types of omics data) having a Spearman's ρ with an absolute value above a threshold number can have an edge between them in the network of correlations. p-values can also be utilized to identify edges between vertices of the network of correlations. In particular implementations, dynamic analytes having p-values greater than 0.05 can be included in the network of correlations.

In certain implementations, the network of correlations can be stored in memory of one or more computing devices. In particular implementations, Pandas data frames or R data frames can be utilized to store the network of correlations. Utilizing Panda data frames or R data frames to store the network of correlations and/or information related to the network of correlations can provide efficient storage and retrieval of the network of correlations and/or information related to the network of correlations. The network of correlations can also be transmitted to one or more computing devices. In various implementations, the network of correlations can be determined utilizing computing devices in a cloud-computing architecture and the network of correlations can be sent from the cloud-computing architecture to one or more computing devices requesting the network of correlations and/or information related to the network of correlations.

The network of correlations can be analyzed on a number of levels. In particular implementations, the network of correlations can include a hierarchy of levels for analysis and the level or levels on which the network of correlations is analyzed can improve the efficiency and accuracy of the analysis. In some implementations, the modularity of the network of correlations can be utilized to determine the level of analysis of the network of correlations. The modularity of the network of correlations can indicate an arrangement of edges that is statistically improbable when compared to an equivalent network with edges placed at random.

### Exemplary Embodiments

1. A method including:

receiving multi-omic data from at least 100 individuals wherein the multi-omic data includes genomic data, and at least one of proteomic data, metabolomic data, microbiome data, transcriptomics data, epigenomic data, or clinical test results data;

calculating genetic risk for a plurality of conditions for the plurality of individuals utilizing the genomic data and genome-wide association studies (GWAS);

normalizing and transforming the genetic risk as well as the multi-omic data into comparable data vectors;

statistically analyzing the comparable data vectors and genetic risk scores;

detecting one or more of:

(i) a dynamic analyte that is correlated with (a) a genetic risk for a biological condition or (b) a biological condition, wherein the dynamic analyte was not previously known to correlate with the genetic risk for the biological condition or the biological condition in GWAS at the time of the detecting;

(ii) a dynamic analyte that is correlated with a genetic risk for a biological condition wherein the dynamic analyte is indicative of the genetic risk before the biological condition emerges in an individual;

(iii) a dynamic analyte that is more strongly correlated with a biological condition or genetic risk for the biological condition than previously-identified dynamic analytes or clinical test results correlated with the condition or genetic risk for the biological condition;

(iv) up- or down-regulation of a dynamic analyte before a biological condition emerges in an individual, wherein the up- or down-regulation of the dynamic analyte was previously only known to be associated with the developed biological condition; and

(v) susceptibility of an individual to an intervention based on the genetic risk of the individual for a biological condition and level of a dynamic analyte associated with the genetic risk for the biological condition.

2. A method of embodiment 1, wherein the transforming includes at least one of mean-centering or standard deviation scaling.
3. A method of embodiment 1, wherein the transforming includes at least one of:

log transformation,

exponentiation, or

determining differences between values of at least one dynamic analyte at multiple points in time.

4. A method of any of embodiments 1-3, wherein the transformation includes combining multiple dynamic analytes into a single analyte through a linear or nonlinear combination.
5. A method of any of embodiments 1-4, wherein the analyzing includes performing linear regression.
6. A method of any of embodiments 1-4, wherein the analyzing includes performing nonlinear regression.
7. A method of any of embodiments 1-6 wherein the genomic data includes whole genome sequence data.
8. A method of any of embodiments 1-7, wherein the microbiome data includes 16s rRNA sequencing data.
9. A method of any of embodiments 1-8, wherein the microbiome data includes full metagenomics or metatranscriptomics sequencing.
10. A method of any of embodiments 1-9, wherein the statistical analysis is performed independently at the phylum, class, order, family, genus, species, and OTU taxonomic levels.
11. A method any of embodiments 1-10, wherein the dynamic analyte data includes metabolomics data and the normalizing includes scaling across samples.
12. A method of any of embodiments 1-11, wherein GWAS studies are excluded based on one or more of: a sample size of less than 5000 individuals; description of genetic risk associated with less 5 or fewer SNVs; or lack of at least one SNV with a p-value of <10-e8.
13. A method of any of embodiments 1-12, wherein the calculating the genetic risk includes: for each variant of one or more variants associated with a trait multiplying a number of effect alleles by an effect size to determine a respective score to produce a plurality of scores; and summing the plurality of scores for the trait to produce a polygenic score for the individual.
14. A method of embodiment 13, wherein the one or more variants are derived from a single published study.
15. A method of embodiment 13, wherein the one or more variants are derived from multiple independent studies investigating the trait.
16. A method of embodiment 13, wherein the one or more variants include a subset of variants from a single published study.
17. A method to identify dynamic analytes indicative of a genetic risk for a biological condition before the biological condition emerges including:

receiving multi-omic data including genome sequence data sets and dynamic analyte data sets from a plurality of individuals wherein a subset of the plurality of individuals have the genetic risk for the biological condition and the subset of the plurality of individuals do not have the genetic risk for the biological condition;

calculating genetic risk for the biological condition for the plurality of individuals utilizing genomic data and genome-wide association studies (GWAS);

normalizing and transforming the genome sequence data sets and dynamic analyte data sets into comparable data vectors;

statistically analyzing the comparable data vectors; and

detecting at least one dynamic analyte whose up- or down-regulation is correlated with the genetic risk before the biological condition emerges in the plurality of individuals.

18. A method of embodiment 17, wherein the transforming includes mean-centering and standard deviation scaling.
19. A method of embodiment 17 or 18, wherein the analyzing includes performing regression.
20. A method of any of embodiments 17-19, wherein the analyzing includes calculating Spearman's ρ following data partitioning by dynamic analyte type.
21. A method of claim any of embodiments 17-20, wherein the genome sequence data sets include whole genome sequence data for each individual of the plurality of individuals.
22. A method of any of embodiments 17-21, wherein the dynamic analyte data includes at least one of clinical laboratory test data, proteomic data, metabolomics data, or microbiome data.
23. A method of any of embodiments 17-22, wherein the dynamic analyte data includes proteomics data from plasma depleted of the 14 most abundant plasma proteins per sample.
24. A method of any of embodiments 17-23, wherein the dynamic analyte data includes proteomics data and the normalizing includes extension positive control and negative control Cq values.
25. A method of any of embodiments 17-24, wherein the dynamic analyte data includes gut microbiome data.
26. A method of any of embodiments 17-25, wherein the gut microbiome data includes 16s rRNA sequencing data.
27. A method of embodiment 26, wherein the gut microbiome data includes full metagenomics sequencing.
28. A method of any of embodiments 17-27, wherein the statistical analysis is performed independently at the domain, phylum, class, order, and family, genus, species, and OTU taxonomic levels.
29. A method of any of embodiments 17-28, wherein the dynamic analyte data includes metabolomics data and the normalizing includes scaling across samples.
30. A method of any of embodiments 17-29, wherein GWAS studies are excluded based on one or more of: a sample size of less than 5000 individuals; description of genetic risk associated with less 5 or fewer SNVs; or lack of at least one SNV with a p-value of <10-e8.
31. A method to identify dynamic analytes that are more strongly correlated with a biological condition or genetic risk for the biological condition than previously identified dynamic analytes correlated with the genetic risk or the biological condition including:

receiving multi-omic data including g genome sequence data sets and dynamic analyte data sets from a plurality of individuals with the genetic risk for the biological condition;

calculating the genetic risk for the biological condition for the plurality of individuals utilizing genomic data and genome-wide association studies (GWAS);

normalizing and transforming the genome sequence data sets and dynamic analyte data sets into comparable data vectors;

statistically analyzing the comparable data vectors; and

detecting at least one dynamic analyte that is more strongly correlated with the genetic risk than previously identified dynamic analytes correlated with the genetic risk or the biological condition.

32. A method of embodiment 31, wherein the transforming includes mean-centering and standard deviation scaling.
33. A method of embodiment 31 or 32, wherein the analyzing includes performing regression.
34. A method of any of embodiments 31-33, wherein the analyzing includes calculating Spearman's ρ following data partitioning by dynamic analyte type.
35. A method of any of embodiments 31-34, wherein the genome sequence data sets include whole genome sequence data for each individual.
36. A method of any of embodiments 31-35, wherein the dynamic analyte data includes at least one of clinical laboratory test data, proteomic data, metabolomics data, or microbiome data.
37. A method of any of embodiments 31-36, wherein the dynamic analyte data includes proteomics data from plasma depleted of the 14 most abundant plasma proteins per sample.
38. A method of any of embodiments 31-37, wherein the dynamic analyte data includes proteomics data and the normalizing includes extension positive control and negative control C_{q} values.
39. A method of any of embodiments 31-38, wherein the dynamic analyte data includes gut microbiome data.
40. A method of claim 39, wherein the gut microbiome data includes 16s rRNA sequencing data.
41. A method of any of embodiments 31-40, wherein the statistical analysis is performed independently at the phylum, class, order, and family taxonomic levels.
42. A method of any of embodiments 31-41, wherein the dynamic analyte data includes metabolomics data and the normalizing includes scaling across samples.
43. A method of any of embodiments 31-42, wherein GWAS studies are excluded based on one or more of: a sample size of less than 5000 individuals; description of genetic risk associated with less 5 or fewer SNVs; or lack of at least one SNV with a p-value of <10-e8.
44. A method including:

determining a genetic risk associated with a biological condition;

determining dynamic analytes associated with the genetic risk of the biological condition in genome-wide association studies (GWAS);

receiving genome sequence data and dynamic analyte data associated with a plurality of individuals; and

analyzing the genome sequence data and the dynamic analyte data from the plurality of individuals to identify a dynamic analyte that is correlated with the genetic risk associated with the biological condition, wherein the dynamic analyte is different from the dynamic analytes associated with the genetic risk of the biological condition in GWAS.

45. A method of embodiment 44, wherein the dynamic analytes include any one or more of: clinical test results; microbiome measurements; metabolome measurements; or proteome measurements.
46. A method of embodiment 44 or 45, wherein analyzing the genome sequence data and the dynamic analytes data includes performing regression.
47. A method including:

determining a dynamic analyte that is correlated with a genetic risk associated with a biological condition;

receiving genome sequence data and dynamic analyte data associated with an individual;

analyzing the genome sequence data associated with the individual to determine a genetic risk of the individual developing the biological condition;

analyzing the dynamic analyte data associated with the individual to determine whether the dynamic analyte is present within the individual at a level commensurate with the individual's genetic risk or at a level not commensurate with the individual's genetic risk; and

implementing a behavioral or environmental intervention if the level is not commensurate with the individual's genetic risk in a direction that decreases risk of an adverse outcome.

48. A method of embodiment 47, wherein the dynamic analyte data includes any one or more of: clinical test results; microbiome measurements; metabolome measurements; or proteome measurements.
49. A method of embodiment 47 or 48, wherein at least a portion of the dynamic analyte data is obtained by performing a procedure that detects the presence of the dynamic analyte or an amount of the dynamic analyte by binding the dynamic analyte to a molecule or ion and detecting the binding between the dynamic analyte and the molecule or ion.
50. A method of embodiment 49, wherein the molecule includes a series of nucleotides or a series of amino acids.
51. A method of embodiment 50, wherein the molecule includes deoxyribonucleic acid (DNA) or ribonucleic acid (RNA).
52. A method of any one of embodiments 49-51, wherein the dynamic analyte is a protein and the procedure includes performing an assay that includes probes to bind a number of proteins.
53. A method of embodiments 49, wherein the dynamic analyte data includes microbiome data and the procedure includes binding at least a portion of the 16S ribosomal RNA (rRNA) to a series of nucleotides and performing polymerase chain reaction (PCR).
54. A method of any one of embodiments 49-53, wherein the dynamic analyte data includes proteomics data from plasma depleted of the 14 most abundant plasma proteins per sample.
55. A method of preserving wellness in a population including:

receiving dynamic analyte data from individuals in a population wherein the dynamic analyte data is collected from the individuals at multiple time points;

monitoring the dynamic analyte data for changes in dynamic analytes within individuals that indicate emergence of a biological condition in the individuals;

initiating a treatment in the individuals when the monitoring reveals the change;

thereby preventing development of the biological condition and preserving wellness in the population.

56. A method of embodiment 55, wherein the dynamic analyte data includes proteomic data, metabolomics data, microbiome data, and clinical test results data.
57. A method of embodiment 55 or 56, further including receiving genome sequence data from the individuals in the population.
58. A method including:

determining a genetic risk set associated with a biological condition;

determining a particular dynamic analyte found in elevated or reduced levels in individuals having the particular condition as compared to individuals not having the biological condition;

receiving genome sequence data associated with a plurality of individuals, wherein each individual of the plurality of individuals shows no symptoms of the biological condition;

analyzing the genome sequence data to identify a set of individuals of the plurality of individuals, wherein each individual of the set of individuals has a genetic risk of developing the biological condition;

monitoring the set of individuals to determine if and when one or more individuals of the set of individuals develops the biological condition;

receiving dynamic analyte data associated with particular individuals in the set of individuals, wherein the dynamic analyte data includes dynamic analyte data associated with multiple samples from each of the particular individuals over a period of time;

analyzing the received dynamic analyte data to identify the dynamic analyte;

determining, based at least in part on analyzing the dynamic analyte data and on the monitoring, whether the dynamic analyte increases or decreases before or after onset of the biological condition.

59. A method of embodiment 58, wherein determining the genetic risk set associated with the biological condition includes analyzing data from genome-wide association studies (GWAS).
60. A method of embodiment 58 or 59, wherein analyzing the genome sequence data to identify the set of individuals that have a genetic risk of developing the biological condition includes determining whether the genetic risk of the individual developing the biolgoical condition is greater than a threshold genetic risk.
61. A method of any of embodiments 58-60, wherein the dynamic analyte data includes any one or more of: clinical test results; microbiome measurements; metabolome measurements; or proteome measurements.
62. A computing system including:
one or more processors; and
non-transitory memory including computer-readable instructions that when executed by the one or more processors perform operations including:
   obtaining clinical testing data for a plurality of clinical tests;
   obtaining biological data for a plurality of individuals, the biological data including a plurality of dynamic analytes;
   analyzing the clinical testing data and the biological data to determine respective correlations between at least one of (1) pairs of clinical tests of the plurality of clinical tests, (2) pairs of dynamic analytes of the plurality of dynamic analytes, or (3) pairs of a respective clinical test of the plurality of clinical tests and a respective dynamic analyte of the plurality of dynamic analytes;
   generating a network of correlations that includes at least a portion of the respective correlations, the network of correlations including vertices and edges between respective pairs of the vertices, each vertex of the vertices corresponding to a clinical test of the plurality of clinical tests or a dynamic analyte of the plurality of dynamic analytes;
   determining that an individual of the plurality of individuals is asymptomatic with respect to a biological condition;
   performing an analysis of the biological data of the individual with respect to the network of correlations; and
   determining a probability the individual will exhibit one or more phenotypes of the biological condition based at least partly on the analysis.
      63. The computing system of embodiment 62, wherein the operations further include:
determining that the probability the individual will exhibit the one or more phenotypes of the biological condition is greater than a threshold probability; and
determining an intervention from a plurality of interventions, wherein the intervention is designed to reduce the probability that the individual will exhibit the one or more phenotypes of the biological condition.
   64. The computing system of embodiment 62 or 63, wherein the correlation for each pair of the number of vertices of the network is above a threshold.
   65. The computing system of any one of embodiments 62-64, further including a data store storing the clinical testing data and the biological data for the plurality of individuals in the data store.
   66. The computing system of any one of embodiments 62-65, wherein the biological data includes genomic data, proteomic data, metabolomics data, gut microbiome data, or combinations thereof.
   67. The computing system of any one of embodiments 62-66, wherein the plurality of dynamic analytes include one or more metabolites, one or more proteins, at least a portion of respective genomes of the plurality of individuals, at least a portion of respective microbiomes of the plurality of individuals, or combinations thereof
   68. The computing system of any one of embodiments 62-67, wherein the operations further include determining a group including at least one of (1) one or more vertices of the network of correlations related to the biological condition or (2) one or more edges of the network of correlations related to the biological condition.
   69. The computing system of any one of embodiments 62-68, wherein the analysis is performed based at least partly on a hierarchical level of the network of correlations determined based at partly on a modularity of the network of correlations, the modularity corresponding to an arrangement of edges of the network of correlations that is statistically improbably in relation to an equivalent network with edges placed at random.
   70. The computing system of any one of embodiments 62-69, wherein the analysis of the network of correlations includes determining Spearman's ρ for each of the at least one of at least one of (1) pairs of clinical tests of the plurality of clinical tests, (2) pairs of dynamic analytes of the plurality of dynamic analytes, or (3) pairs of a respective clinical test of the plurality of clinical tests and a respective dynamic analyte of the plurality of dynamic analytes.
   71. The computing system of any one of embodiments 62-70, wherein the analysis of the network of correlations includes: for individual edges of the network of correlations, calculating a number of weighted shortest paths from all vertices to all other vertices that pass over an individual edge and removing edges that are associated with at least a threshold number of weighted shortest paths.
   72. A computer-implemented method including:
obtaining, by a computing device including a processor and memory, clinical testing data for a plurality of clinical tests;
obtaining, by the computing device, biological data for a plurality of individuals, the biological data including a plurality of dynamic analytes and the plurality of individuals are asymptomatic with respect to a biological condition;
analyzing, by the computing device, the clinical testing data and the biological data to determine respective correlations between at least one of (1) pairs of clinical tests of the plurality of clinical tests, (2) pairs of dynamic analytes of the plurality of dynamic analytes, or (3) pairs of a respective clinical test of the plurality of clinical tests and a respective dynamic analyte of the plurality of biological indicators;
generating, by the computing device, a network of correlations that includes at least a portion of the respective correlations, the network of correlations including vertices and edges between respective pairs of the vertices, each vertex of the vertices corresponding to a clinical test of the plurality of clinical tests or a dynamic analyte of the plurality of dynamic analytes;
determining, by the computing device, a number of pre-existing dynamic analytes for the biological condition based at least partly on data obtained from an additional plurality of individuals that exhibited one or more phenotypes of the biological condition; and
determining, by the computing device, one or more additional dynamic analytes for the biological condition based at least partly on the network of correlations.
   73. The method of embodiment 72, wherein the one or more additional dynamic analytes are not associated with the biological condition in previously published literature and the pre-existing dynamic analytes are included in the previously published literature.
   74. The method of embodiment 72 or 73, further including determining one or more parameters for a clinical trial regarding an intervention for the biological condition, wherein the intervention regulates the one or more additional dynamic analytes.
   75. The method of any one of embodiments 72-74, wherein the biological condition is Alzheimer's disease and the one or more additional dynamic analytes include matrix metalloproteinase-2 (MMP2) and the one or more pre-existing dynamic analytes include amyloid β.
   76. The method of any one of embodiments 72-75, wherein the biological condition is insulin resistance and the one or more additional dynamic analytes includes gamma-glutamyltyrosine.

Experimental Examples. Procedures for the P100 were run under the Western Institutional Review Board (IRB Protocol Number 20121979) at the Institute for Systems Biology (ISB). Blood was collected during three two-week intervals (‘rounds’) spaced every three months. Participants completed their blood draw during each 2-week interval as their schedule permitted. Urine, stool and saliva samples were also collected during each round. Blood samples from each participant were collected and processed using the proper collection and processing tubes, outlined by Genova Diagnostics and Quest Diagnostics and described below, and couriered to the testing facilities to maintain maximum sample stability. Participants were asked to fast for 12 hours before all blood collections. A 99.3% compliance rate in fasting was observed. Additional whole blood and plasma samples were collected from participants and shipped to BioStorage Technologies, an international CAP-accredited biorepository. Additional samples were used for metabolomics (Metabolon), SRM proteomics (ISB), Olink Proseek protein panels (ISB) and whole genome sequencing (Complete Genomics and the New York Genome Center). Participants collected stool samples at home for 16S rRNA sequencing (Second Genome), and provided activity data through quantified-self devices (Fitbit).

Clinical Laboratory Tests. For Genova, a total of one urine tube and nine blood tubes were collected. The blood tubes included two Na-Heparin Trace Element tubes, three SST tubes, three EDTA purple top tubes, and one NMR black-top LipoTube. First morning void urine was collected in the Genova provided green-top tube by participants the morning of their blood draw. Urine was sent frozen to Genova. Both Na-Heparin tubes were spun for 15 minutes at 3000 rpm. The plasma from one Na-Heparin tube was transferred to a blue-top preservative tube provided by Genova and shaken and spun for 5 minutes at 2500 rpm. Supernatant was then transferred to the yellow top transfer tube provide by Genova and shipped frozen. Plasma from the second Na-Heparin tube was transferred to an amber top transfer tube and shipped frozen. Each SST tube was left to clot for 15 minutes then spun for 15 minutes at 3000 rpm. The plasma was for all three was pipetted to transfer tubes and shipped frozen. All three EDTA-lavender top tubes were refrigerated after collection and shipped refrigerated. The single NMR black-top LipoTube was clotted for 30 minutes then spun for 15 minutes at 3000 rpm. The specimen was left in the tube and shipped refrigerated.

Each saliva collection included four samples within a single day (four-point cortisol test). For collection of the four saliva samples, participants were instructed to abstain from eating or drinking 30 minutes prior to each collection. All participants were given the following collection times for each of their four samples: Sample 1: Collect before breakfast, between 7 am-9 am and one hour after waking up. Sample 2: Collect before lunch, between 11 am-1 pm. Sample 3: Collect before dinner, between 3 pm-5 pm. Sample 4: Collect before bedtime, between 10 pm-12 am. All samples were frozen overnight after collection and shipped directly to Genova.

Two SST tubes were collected for Quest Diagnostics. After collection, the two tubes were left to clot for 15 minutes and then spun for 15 minutes at 3000 rpm. Samples were left in the tube and shipped at ambient temperature.

Whole Genome Sequencing. Participant whole blood samples were submitted to either Complete Genomics Inc. (41 participants) or the New York Genome Center (67 participants) for whole genome sequencing (WGS). Complete Genomics conducted the whole genome sequencing using their standard complete sequencing platform employing high-density DNA nanoarrays populated with DNA nanoballs for 40× average coverage. The New York Genome Center used Illumina's 2×150 bp HiSeq X technology for 30× average coverage, using TruSeq kits for library prep. Both vendors aligned sequenced reads to human reference sequence GRCh37/hg19. NYGC used BWA v0.7.8-r455.

Complete Genomics provided a vcfBeta file for each sequenced sample calculated with CGAPipeline v2.5.0.20. NYGC provided a VCF4.1 file for each sequenced sample calculated with GATK HaplotypeCaller, following duplicate marking with Picard v1.83, and indel realignment and base quality recalibration. GATK v3.1.1-g07a4bf8 was used for BAM file post-processing and variant calling. Only variants with a FILTER value of PASS were used in downstream analyses for both CGI and Illumina data. Copy number variant status was determined using Reference Coverage Profiles (Glusman, et al., (2015). Front Genet 6, 45.). Variant frequencies were annotated using Kaviar (Glusman, et al., (2011). Bioinformatics 27, 3216-3217.). For comparison of the two technologies, monozygotic twins sequenced using separate technologies were used. 99.12% concordance in variant calls across technologies in 6601 distinct loci from the GWAS catalog were observed, while 0.21% were fully observed and discordant. Table 1 lists the full statistics of this comparison.

TABLE 1
Concordance of 6601 loci between monozygotic
twins sequenced on Illumina and CGI

| Count | Percent | Description |
|---|---|---|
| 6543 | 99.12% | Fully observed and concordant between Illumina and |
| | | CGI |
| 13 | 0.20% | Partially observed in CGI but compatible with Illumina |
| 29 | 0.44% | NOCALL in CGI |
| 2 | 0.03% | NOCALL in Illumina |
| 14 | 0.21% | Fully observed and discordant between Illumina and |
| | | CGI |

Gut Microbiome 16S rRNA Sequencing. Gut microbiome data in the form of 16S OTU (Operational Taxonomic Unit) read counts were provided by Second Genome. 250 bp paired end MiSeq profiling of the 16S v4 region was performed as described previously (Caporaso, et al., (2012). Isme J 6, 1621-1624.), with 50,000-150,000 reads generated per sample. 16S sequence clustering and open reference OTU picking (Rideout, et al., (2014). PeerJ 2, e545) were performed using USEARCH with a proprietary strain database. Each OTU was then represented as a fraction of an individual's total microbiome composition. These OTU proportions were placed in a vendor provided taxonomy and aggregated at the kingdom, phylum, class, order, family, genus, and species levels (See Table 2 below). α-diversity (Whittaker (1972). Taxon 21, 213), a measure of the number of OTUs observed as well as the evenness of their distributions, was calculated as the within-sample Shannon diversity index:

$${H}_{j}^{′} = - {\sum }_{i}^{ } {p}_{ij} ln ( {p}_{ij} )$$

where pᵢⱼ is the relative abundance of OTU i in sample j.

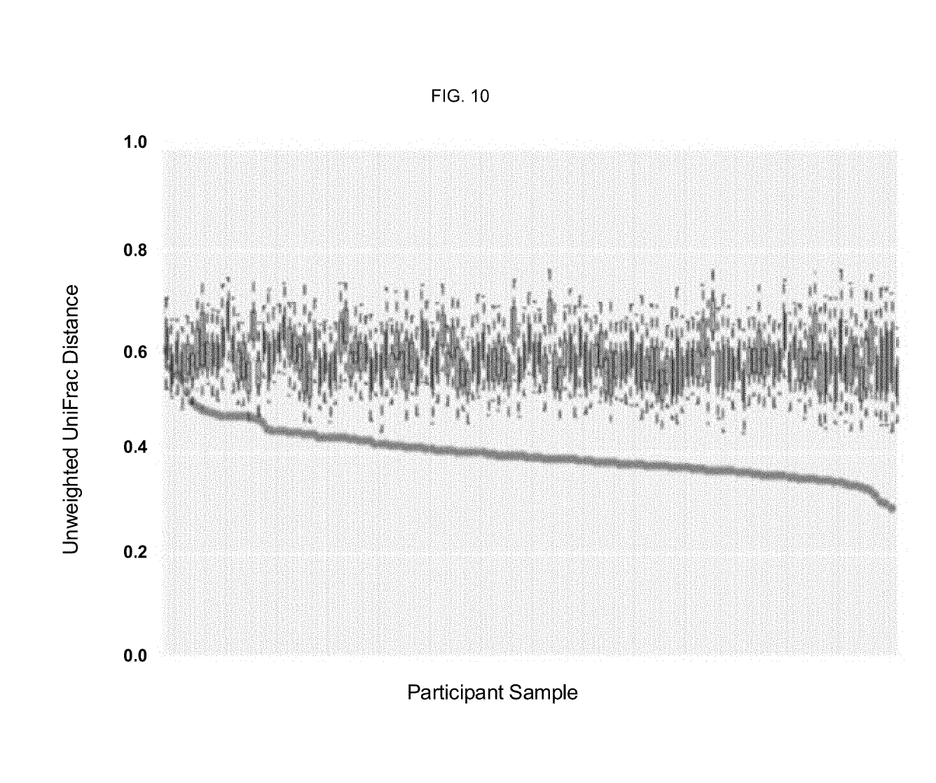

For the inter-individual comparisons (See FIG. 10), representative sequences were aligned using PyNAST 1.2.2 (Caporaso et al., 2010a) via QIIME 1.9.1 (Caporaso et al., 2010b) with the Greengenes (McDonald et al., 2012) 85% OTU representative sequences as a template. The alignment was filtered to remove high entropy positions using the Lane mask (Lane, 1991). A phylogeny was reconstructed using FastTree 2.1.7. Unweighted UniFrac distances (Hamady et al., 2010; Lozupone and Knight, 2005; Lozupone et al., 2011) were computed on the table using QIIME. scikit-bio 0.2.3 (http://scikit-bio.org) was used in a custom Jupyter Notebook (Perez and Granger, 2007) with matplotlib (Hunter, 2007) and seaborn (Botvinnik et al., 2016) to process the distance matrix. Specifically, for each sample, the distance between it and the participant's successive time point was determined (the grey points in FIG. 10). All of the distances from that sample to all other samples at the successive time point were retrieved (the box-whisker plots in FIG. 10). Subsequent statistics were computed using SciPy 0.17.0 (Jones, et al., (2015). SciPy: Open source scientific tools for Python, 2001—(URL http://www.scipy.org)).

The proprietary strain database used for microbiome analyses can be downloaded from Second Genome.

TABLE 2
Number of unique taxa observed for each
taxonomic level across participants

| Domain | Phylum | Class | Order | Family | Genus | Species | OTU |
|---|---|---|---|---|---|---|---|
| 2 | 13 | 27 | 52 | 205 | 779 | 1275 | 4616 |

Metabolomics. Metabolon Inc. conducted the metabolomics assays on participant plasma samples at three time points for each participant throughout the course of the study. Metabolon Inc. generated the data using their DiscoveryHD4 platform in addition to their Fatty Acid Metabolism (FAME) panel that use a combination of ultra-high-performance liquid chromatography with tandem mass spectrometry (MS) and gas chromatography (GC) in the identification of metabolites and fatty acids. The metabolite values were reported relative to their concentrations among all participants, except for lipids that were measured via GC-FID, which were reported as molar percentages of each participant's total fatty acids. For analysis, the metabolomics data was median scaled, such that the median value for each metabolite was one and values that fell beneath the range of detection were imputed to be the minimum observed value. This scaling was performed across all samples. All time points were run as a single batch. Counts of metabolites detected using each technology are listed in Table 3.

TABLE 3
Number of metabolites observed by detection method

| GC-FID | GC/MS | LC/MS(neg) | LC/MS(pos) | LC/MS(polar) |
|---|---|---|---|---|
| 34 | 29 | 347 | 159 | 74 |

Protein levels in plasma were determined by Proximity Extension Assays using two Olink (Uppsala, Sweden) Proseek Multiplex 96×96 kits and quantified by real-time PCR using the Fluidigm (South San Francisco, Calif.) BioMark HD system. Each kit provides a microtitre plate for measuring 92 protein biomarkers in 90 samples. Each well contains 96 pairs of DNA-labeled antibody probes. When a matched pair of probes bind to their target protein, their DNA labels are brought into close proximity and a PCR target sequence is formed by a proximity-dependent DNA polymerization. One plate contains 96 wells for processing 90 samples, 3 positive controls, and 3 negative controls to determine the lower detection limit. Each sample is also spiked with four controls to monitor variation in the three steps of the PEA process. Two non-human antigens serve as incubation controls, one DNA-labeled antibody serves as an extension control, and an oligonucleotide serves as a detection control.

The Proseek cardiovascular (CVD I) and inflammation (Inflammation I) panels target 165 different proteins with 19 overlapping measurements. Plasma samples from 83 subjects drawn at three intervals were assayed. One sample was assayed in triplicate on all plates and additional samples were replicated for a total of 270 multiplex cardiovascular and 270 multiplex inflammation assays. A total of 41,085 data points were collected. Assays were run according to the manufacturer's instructions. In short, 1 μl of each sample was incubated with the antibody probes at 4° C. overnight. After binding, the extension mix was added and the products were extended and amplified using 17 cycles of PCR (Applied Biosystems 9700, Life Technologies, Carlsbad, Calif.). Next, 2.8 μl of each PCR product was added to the detection mix and loaded into the sample wells of a Fluidigm 96.96 Dynamic Array plate (Fluidigm Corporation) while kit primers were loaded into the primer wells. The Dynamic Array was primed in a Fluidigm HX IFC controller and then loaded into the Fluidigm Biomark imaging thermocycler for quantitative PCR. Quantification cycle (Cq) values for each measurement were determined using Fluidigm's Real-Time PCR Analysis software and BiomarkDataCollection version 4.1.3. Data was normalized using the extension positive control and the negative control Cq values. The limit of detection was defined as three times the standard deviation of the negative controls.

Selected Reaction Monitoring (SRM). (i) SRM Assay and Method Development. SRM assays were developed for 200 peptides representing 100 proteins. For each peptide sequence the heavy isotope labeled analogue was synthesized (PEPotec SRM library Grade 1, Thermo-Fisher Scientific, Huntsville, Ala.) with cysteine residues carbamidomethylated and the C-terminal arginine as R[13C6, 15N4] or lysine as K[13C6, 15N2] to allow for relative quantification. The 200 synthetic peptides were individually analyzed on a 6530 accurate-mass Q-TOF liquid chromatography mass spectrometry (LC-MS) system (Agilent Technologies, Santa Clara, Calif.) using a ProtID-Chip-150 (II) (Agilent Technologies, Santa Clara, Calif.) to verify and confirm successful peptide synthesis. The 200 peptides were pooled as internal standard. Multiplexed SRM assays were established with the human SRMAtlas and the synthetic peptides on a 6460 QQQ MS system equipped with Jet Stream ESI technology and a 1290 Series UHPLC (Agilent Technologies, Santa Clara, Calif.). SRM assays were optimized with regard to sensitivity and specificity, and with the aim to target 200 peptides in a single analysis. 1200 transitions were determined, 3 transitions to target each light endogenous peptide and 3 transitions to target each isotope labeled heavy peptide, and peptides separated on a reversed phase column (Zorbax SB-C18, 50 mm×2.1 mm I.D., 1.8 μm dp, Agilent Technologies, Santa Clara, Calif.) using a gradient from 3% to 30.5% acetonitrile/0.1% formic acid/water over 55 min at a flow rate of 0.2 mL/min. Data were acquired in dynamic MRM mode with a fixed cycle time of 2500 ms and a minimum dwell time of 10 ms.

(ii) Plasma Sample Preparation. Plasma samples were thawed on ice and centrifuged for 10 min at 14,000 rpm to separate tissue debris or a lipid layer. 110 μL plasma were depleted from the 14 most abundant plasma proteins using the multiple affinity removal system (MARS Hu-14, 4.6×100 mm, Agilent Technologies, Santa Clara, Calif.) according to the manufacturer's protocol. The depleted fraction was collected in 1.25 mL of MARS14 Buffer A and denatured by adding 600 mg urea to 8 M final concentration. Samples were reduced with 5 mM dithiothreitol for 30 min at 55° C., alkylated with 14 mM iodoacetamide for 30 min at room temperature in darkness and desalted using a GE HiPrep 26/10 column (GE HealthCare Life Sciences, Pittsburgh, Pa.) and 1200 HPLC system (Agilent Technologies, Santa Clara, Calif.). The protein concentration of the desalted samples was determined by bicinchoninic acid assay (BCA) (Thermo-Fisher Scientific, San Jose, Calif.). An aliquot of the pooled 200 synthetic peptides was spiked into an aliquot of each plasma sample (equal protein amounts) prior to the digestions with trypsin (Promega, Madison, Wis.) at 1:50 enzyme:substrate ratio for 16 h at 37° C. Digests were dried under centrifugal vacuum evaporation (Savant, Thermo-Fisher Scientific, San Jose, Calif.) and reconstituted to 1 μg/μL protein concentration.

(iii) Plasma Sample Analysis. 20 μg of each plasma sample spiked with the 200 isotope labeled peptides was subjected to SRM analysis using the method described above. SRM data were analyzed with Skyline (MacLean, et al., (2010). Bioinformatics 26, 966-968). SRM traces were integrated with default settings and manually inspected to verify correct peak assignment and co-elution of endogenous and isotope labeled standard peptides. The relative peptide abundance level was reported as ratio of endogenous light to the heavy standard.

Quantified Self Tracking. Participants were asked to wear a Fitbit activity tracker throughout the nine-month study. Participants were offered either a Fitbit Flex (wrist) or a Fitbit One (clip-on). These Fitbit models measure activity using the number of steps an individual takes each day; they do not measure heart rate. A minimum of 40 days of Fitbit usage was required in order to estimate the average activity for each participant; 64% of the participants met this criterion. The Fitbit device estimates user-specific ‘activity calories’ independently of basal metabolic rate (BMR). For all calculations, only the estimated ‘activity calories’, excluding BMR, was used. These data were used only as a relative indicator of activity levels rather than an absolute measure of caloric burn. Participants were asked to self-record blood pressure and resting heart rate weekly using an automated sphygmomanometer.

Genomic Traits. The National Human Genome Research Institute's GWAS catalog lists results from more than 2000 published studies comprising over 1000 genetic traits (Welter, et al., (2014). Nucleic Acids Res. 42, D1001-D1006). In order to increase the probability of finding a statistically significant correlation that passed multiple hypothesis correction, a strict filtering procedure was applied, excluding studies which did not contain at least one SNV with a p-value <10e-8. Studies which contain few SNVs are likely to produce a vector of cumulative genetic variation with low entropy, where almost all values are identical save a few. Such low entropy measurements are more likely to produce spurious correlations in the relatively small number of samples. All traits associated with five or fewer SNVs were therefore excluded. Furthermore, studies were required to have a sample size of at least 5000 individuals. In the event that multiple studies examined the same trait, the study with the largest sample size was kept. Finally, traits with too-generic descriptions (e.g. ‘common traits’ or ‘metabolic traits’), which did not provide a useful description of the purpose of the original study were manually excluded. The combination of these filters retained 127 genetic traits that were used for further analysis. Three common CNVs were included as additional genetic features computed using Reference Coverage Profiles (Glusman et al., 2015), bringing the total to 130.

Included in the GWAS catalog are the beta-coefficients/odds ratios as well as the p-values for the predicted effect of each variant for that trait based on the association models from the original paper. Two assumptions to simplify the calculation of the genetic effects on each individual were made. First, it was assumed that the beta-coefficients (or log odds ratios) combined in an additive manner based on the number of effect alleles present in each individual. Therefore, if a single effect allele were present the beta-coefficient for that variant was added into the cumulative genetic effect. If two copies of the effect allele were present twice the value of the beta-coefficient was added into the cumulative genetic effect for that individual. The second assumption was that the effects of each variant are independent of the effects of all other variants used in the model, In other words, the values of all interaction terms are zero. These two simplifying assumptions allowed calculation of cumulative ‘genetic effect’ on a given trait for each individual in the study.

There are a number of pitfalls to this approach that served to temper expectations, First, GWAS by definition can only identify variants that occur commonly enough in the population to be associated statistically with a trait. Unless one is able to genotype a substantial fraction of the human population at risk for a particular trait, many rare variants will never rise above the level of noise in a GWAS. Furthermore, because they employ genotyping chips most GWAS ignore copy number variations (CNVs) and other genome features that may have a significant effect on genetic traits. Three common CNVs were included as part of the study as additional genomic features. Genotyping chips are also limited by the number of variants tested on the chip. Finally, many GWAS are applied to cohorts of individuals from similar ancestries to improve their likelihood of discovering associated variants; it is therefore possible that results from these studies do not generalize to individuals from differing ancestral populations.

Coaching, charting, and compliance tracking. The described study was designed as a cohort study, in that participants were assembled and followed forward in time, rather than retrospectively as in a case-control study. However, the study was unusual in that the study actively attempted to modify the participants' behavior throughout the nine-month period. Participants were assigned to a behavioral coach, who walked them through a selected subset of their data and made recommendations on lifestyle changes. These lifestyle changes were recommended in an attempt to alter markers of known clinical significance and/or compensate for genetic predispositions for which reliable published evidence is available. Each participant was eligible for one 30-minute coaching session per month, though participants were not penalized or excluded from the study if they chose not to participate in the coaching sessions. Participants were also able to communicate privately and securely with the coach via a website portal created specifically for this project. Participants also received their data through the website portal. The study collected statistics on participation in the coaching calls, blood draws, and compliance with coaching recommendations.

As previously stated, clients were offered specific coaching recommendations based on their genetics and clinically actionable data. These recommendations were customized prior to each call by the study clinician and coach, in consultation with the study physician. All clinical markers and recommendations were reviewed and approved by the study physician prior to their communication to each participant. While these recommendations were specific to each individual based on their data, they typically fell into one of several major categories, including diet, exercise, stress management, dietary supplements, or physician referral. Coaching focused on four primary quadrants: Cardiovascular, Diabetes, Inflammation, and Nutrition. The clinical tests used to quantify these quadrants are provided in Table 4. Generalized estimating equation (GEE) regression models were used to estimate the average change for each clinical lab by round while controlling for the effects of age and sex. Coefficients, 95% confidence intervals, and p-values for all participants as well as those who began the study out-of-range are listed in Table 5.

TABLE 4
Labs used to analyze changes in the quadrants targeted by coaching

| Cardiovascular | Diabetes | Inflammation | Nutrition |
|---|---|---|---|
| Total cholesterol | Fasting glucose | Interleukin-6 | Vitamin D |
| LDL cholesterol | HbA1c | Interleukin-8 | Glutathione |
| HDL cholesterol | Insulin | TNF-alpha | Ferritin |
| Triglycerides | HOMA-IR | hs-CRP | Zinc |
| LDL pattern | | | Methylmalonic |
| LDL particle number | | | acid |
| LDL medium particle number | | | Selenium |
| LDL small particle number | | | Copper |
| HDL large particle number | | | Manganese |
| | | | Mercury |
| | | | Arachidonic acid |
| | | | EPA |
| | | | DHA |

TABLE 5
Generalized estimating equation (GEE) regression estimates for change in each analyte by round. The coefficient
is an estimate of the average change in the population for that analyte by round adjusted for age and sex.
Each coefficient shown has the unit of the analyte it represents. ‘Out-of-range at baseline’ shows
the estimates using only those participants who were out-of-range for that analyte at the beginning of the
study. NaN values are present where no participants were out-of-range at baseline. ‘All participants’
shows the estimates using all participants in the study. Several analytes are measured by both Quest and
Genova; with the exception of LDL particle number, the direction of effect was concordant across the two
labs. An independence working correlation structure was used in the GEE models.

| | Clinical | Out-of-range at baseline | | | All participants | | |
|---|---|---|---|---|---|---|---|
| Quadrant | laboratory test | Coef. | 95% conf. | Pvalue | Coef. | 95% conf. | Pvalue |
| Nutrition | Vitamin D | 7.1 | [5.8, 8.5] | 0.0e+00 | 6.5 | [5.2, 7.9] | 3.7e−22 |
| Nutrition | Mercury | −0.0022 | [−0.003, −0.0014] | 1.6e−08 | −0.00180 | [−0.0025, −0.0011] | 3.3e−07 |
| Diabetes | HbA1c | −0.086 | [−0.12, −0.048] | 9.1e−06 | −0.047 | [−0.072, −0.023] | 1.5e−04 |
| Cardiovascular | LDL particle | 130 | [64.0, 190.0] | 9.4e−05 | 79 | [44.0, 110.0] | 1.0e−05 |
| | number (Quest) | | | | | | |
| Nutrition | Methylmalonic | −0.51 | [−0.8, −0.22] | 5.1e−04 | 0.012 | [−0.031, 0.054] | 6.0e−01 |
| | acid (Genova) | | | | | | |
| Cardiovascular | LDL pattern | −0.16 | [−0.25, −0.067] | 5.6e−04 | −0.0098 | [−0.047, 0.028] | 6.1e−01 |
| Inflammation | Interleukin-8 | −6.1 | [−9.6, −2.6] | 5.9e−04 | −0.58 | [−1.4, 0.25] | 1.7e−01 |
| Cardiovascular | Total cholesterol | −6.5 | [−10.0, −2.7] | 6.6e−04 | −0.62 | [−3.3, 2.1] | 6.5e−01 |
| | (Quest) | | | | | | |
| Cardiovascular | LDL cholesterol | −4.8 | [−7.7, −2.0] | 7.9e−04 | −1.3 | [−3.4, 0.87] | 2.5e−01 |
| Cardiovascular | LDL particle | −69 | [−110.0, −28.0] | 1.1e−03 | −40 | [−72.0, −6.9] | 1.7e−02 |
| | number (Genova) | | | | | | |
| Cardiovascular | Small LDL | −55 | [−93.0, −17.0] | 4.5e−03 | −37 | [−64.0, −9.8] | 7.6e−03 |
| | particle number | | | | | | |
| | (Genova) | | | | | | |
| Diabetes | Fasting glucose | −1.9 | [−3.3, −0.47] | 8.7e−03 | −1.1 | [−2.0, −0.19] | 1.8e−02 |
| | (Quest) | | | | | | |
| Cardiovascular | Total cholesterol | −5.5 | [−9.7, −1.2] | 1.2e−02 | 0.38 | [−2.2, 3.0] | 7.8e−01 |
| | (Genova) | | | | | | |
| Diabetes | Insulin | −2.2 | [−4.1, −0.37] | 1.8e−02 | −0.65 | [−1.0, −0.27] | 6.7e−04 |
| Inflammation | TNF-alpha | −6.6 | [−12.0, −1.1] | 1.8e−02 | 0.31 | [−0.038, 0.67] | 8.1e−02 |
| Cardiovascular | HDL cholesterol | 4.5 | [0.64, 8.4] | 2.2e−02 | 1.9 | [0.98, 2.7] | 3.6e−05 |
| Diabetes | HOMA-IR | −0.56 | [−1.0, −0.081] | 2.2e−02 | −0.15 | [−0.26, −0.049] | 4.0e−03 |
| Nutrition | Methylmalonic | −42 | [−85.0, 0.43] | 5.2e−02 | −8.8 | [−14.0, −4.0] | 3.0e−04 |
| | acid (Quest) | | | | | | |
| Cardiovascular | Triglycerides | −18 | [−42.0, 6.2] | 1.4e−01 | 1.2 | [−5.7, 8.0] | 7.3e−01 |
| | (Genova) | | | | | | |
| Diabetes | Fasting glucose | −0.96 | [−2.3, 0.36] | 1.5e−01 | −0.28 | [−1.1, 0.51] | 4.8e−01 |
| | (Genova) | | | | | | |
| Inflammation | HS-CRP | −0.48 | [−1.2, 0.27] | 2.1e−01 | −0.092 | [−0.47, 0.29] | 6.4e−01 |
| Nutrition | Arachidonic | 0.21 | [−0.14, 0.56] | 2.3e−01 | −0.22 | [−0.4, −0.041] | 1.6e−02 |
| | Acid | | | | | | |
| Cardiovascular | Triglycerides | −14 | [−37.0, 9.5] | 2.5e−01 | −0.47 | [−7.9, 6.9] | 9.0e−01 |
| | (Quest) | | | | | | |
| Nutrition | Zinc | −0.83 | [−2.4, 0.74] | 3.0e−01 | −0.36 | [−0.49, −0.24] | 1.8e−08 |
| Nutrition | Ferritin | −14 | [−42.0, 13.0] | 3.1e−01 | −5.1 | [−9.7, −0.49] | 3.0e−02 |
| Nutrition | Glutathione | 10 | [−9.8, 30.0] | 3.2e−01 | 0.14 | [−20.0, 20.0] | 9.9e−01 |
| Inflammation | Interleukin-6 | −1 | [−3.6, 1.5] | 4.3e−01 | 0.13 | [−0.055, 0.31] | 1.7e−01 |
| Cardiovascular | HDL large | 210 | [−400.0, 810.0] | 5.0e−01 | 100 | [−67.0, 270.0] | 2.3e−01 |
| | particle number | | | | | | |
| Nutrition | Copper | 0.0075 | [−0.015, 0.03] | 5.2e−01 | 0.00085 | [−0.0052, 0.0069] | 7.8e−01 |
| Nutrition | Selenium | 0.034 | [−0.1, 0.17] | 6.3e−01 | 0.015 | [−0.0026, 0.033] | 9.4e−02 |
| Cardiovascular | Medium LDL | 2.8 | [−27.0, 32.0] | 8.5e−01 | 21 | [13.0, 30.0] | 2.4e−07 |
| | particle number | | | | | | |
| Nutrition | Manganese | NaN | NaN | NaN | −0.00071 | [−0.0012, −0.00025] | 2.4e−03 |
| Nutrition | EPA | NaN | NaN | NaN | 0.042 | [−0.043, 0.13] | 3.3e−01 |
| Nutrition | DHA | NaN | NaN | NaN | −0.067 | [−0.11, −0.024] | 2.1e−03 |

Each dataset was transformed into comparable data vectors for statistical analysis. All measurements were mean centered and scaled by the standard deviations of the observed measurements. For correlations the mean analyte value across rounds for each participant was used. The microbiome measurements were compared independently at the phylum, class, order and family taxonomic levels. With the exception of the median-scaled metabolomics data, missing data were not imputed; participants that had a missing value were dropped from pairwise comparisons utilizing that value. Each analyte was age- and/or sex-corrected if a trimmed mean robust regression identified a significant relationship (p<0.01) between age or sex and the dependent variable. In this case the residuals of the model were used in place of the original observations.

Correlation Network. In order to explore the inter-relatedness of the data sources, correlation networks for all pairs of data sources were generated. For the purposes of the correlation network, the data was partitioned into five buckets; clinical laboratory tests, proteomics, metabolomics, genomic traits, and microbiome.

To identify correlations between the normalized data types, Spearman's ρ, which returns a coefficient between −1 and +1, where −1 is perfect negative correlation and +1 is perfect positive correlation between two ranked variables was used. Although widely used, Pearson's r is biased when outliers are present, and being a parametric test it is sensitive to the distribution of the variables being compared. In contrast, Spearman's ρ is a non-parametric test that works on the ranks and is therefore robust to outliers as well as insensitive to the distributions of the variables being compared. While mutual information (MI) has been used in other cases of network inference, it was decided the limited number of samples was too few to properly model the probability distributions. Future studies with larger number of samples will incorporate mutual information in addition to or in place of these standard correlation algorithms.

For each pairwise set of data (e.g. clinical tests vs. proteomics, clinical tests vs. metabolomics, etc.), each measurement from the first dataset was correlated with every measurement from the second dataset using Spearman's ρ. Once the coefficients and p-values were computed, all p-values were adjusted for multiple hypothesis testing using the method of Benjamini and Hochberg (Benjamini and Hochberg, 1995); an adjusted p-value cutoff of 0.05 was chosen as the significance level. The resulting network was used as an input for community analysis described below. Only inter-omic correlations were used for community analysis. 3470 statistically significant correlations were calculated.

Community analysis was performed using the method of Girvan and Newman (Girvan and Newman, 2002). This method involves iteratively calculating edge betweenness centrality on a network: the number of weighted shortest paths from all vertices to all other vertices that pass over that edge. After each iteration, the edge(s) with the highest betweenness centrality are removed and the process is repeated until only individual nodes remain. The visualization of the entire process can be represented as a dendrogram.

Community analysis forms a dendrogram that can be analyzed at multiple hierarchical levels. The network was analyzed at a cut level determined using an unbiased method, the modularity of the community structure (Newman, 2006). Briefly, modularity of community structure corresponds to an arrangement of edges which is statistically improbable when compared to an equivalent network with edges placed at random. Quoting from Newman, ‘the modularity is, up to a multiplicative constant, the number of edges falling within groups minus the expected number in an equivalent network with edges placed at random.’ At every iteration of the community analysis, the modularity was computed, and the communities were analyzed at the iteration which maximized this quantity. The plot of modularity vs. iteration is shown in FIG. 4.

Software packages. All pairwise statistical tests (Spearman) were performed using the Python scipy.stats package (v0.14) (Jones et al., 2015). All linear models were generated using the Python Statsmodels package (v0.6) (Seabold & Perktold (2010). Statsmodels: Econometric and statistical modeling with python). Correlation network p-values were adjusted for multiple hypothesis using the Benjamini-Hochberg (Benjamini and Hochberg, 1995) method via the Python Statsmodels package (v0.6) for each inter-datatype comparison. Community analysis was performed in Python with the NetworkX (Schult & Swart (2008). Exploring network structure, dynamics, and function using NetworkX) package using custom code.

Results. 108 individuals (age 21-89+ years; 59% males, 41% females; 89% Caucasian; not recruited based on any specific phenotype) participated in an IRB-approved study that included whole genome sequencing, and analysis of blood, urine, stool, and saliva samples collected every three months. Each of these collection periods were defined as “rounds”. These samples were used to measure 218 clinical laboratory tests, 643 metabolites, 262 proteins, and 4616 operational taxonomic units (OTUs) in the gut microbiome. The genome was incorporated through a set of 130 genetic traits primarily corresponding to polygenic scores for diseases and quantitative traits based on previous studies. Participants recorded weight, blood pressure and resting heart rate weekly, and performed daily tracking of activity using wearables (Fitbit). The details of correlations across the data types and the breakdown of the correlation network into data community structures are first presented, followed by specific results around a subset of these correlations in the communities.

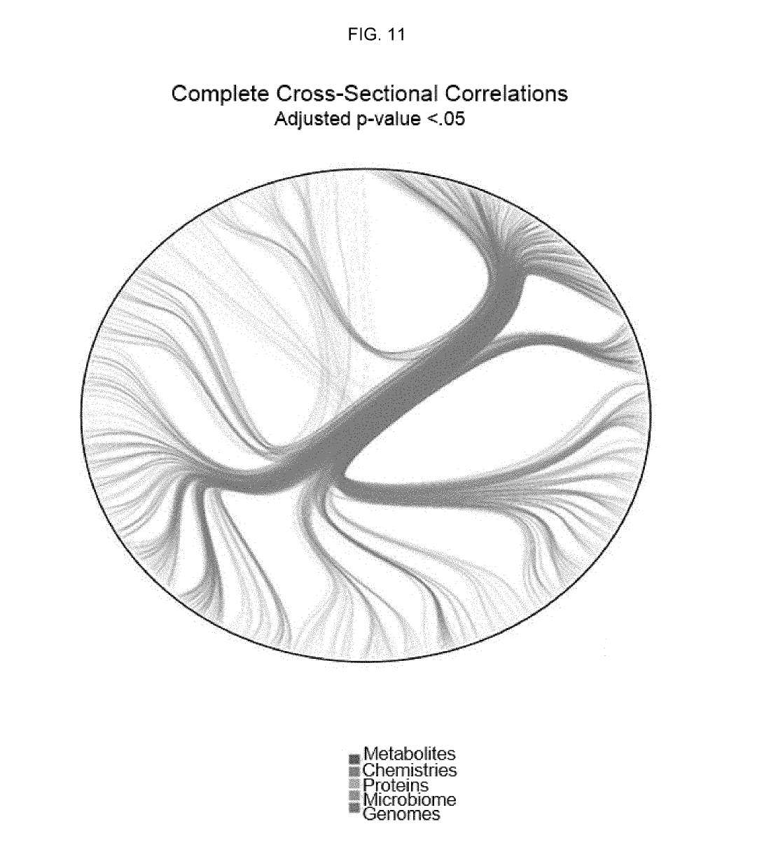

Multi-omic data from 108 participants was studied individually and as a cohort to identify significant associations across data types. An age- and sex-adjusted correlation network was built based on Spearman correlations. In this network, vertices (V) correspond to analytes, and an edge (E) exists between two vertices if and only if a significant (p_{adj}<0.05) correlation was observed after correction for multiple hypotheses (Benjamini and Hochberg, 1995). The correlation network contains 766 nodes and 3,470 edges. The majority of all edges involved a metabolite (3,309) or a clinical laboratory test (3,366), with an additional 20 edges involving the 130 tested genetic traits, 46 with microbiome taxa, and 207 with quantified proteins. A network of cross-correlations is shown in FIG. 11. FIG. 11 shows a cross-correlation network with statistically-significant Spearman correlations (padj<0.05) between all datasets collected in the cohort.

The Metabolites included in the network of FIG. 11 can include one or more of Asparagine, N Acetylalanine, Alanine, N Acetylaspartate (naa), Alpha Hydroxyisovaleroyl Carnitine, Isovalerylcarnitine, Valine, Ethylmalonate, N Acetylvaline, Beta Hydroxyisovaleroxlcarnitine, 3 Methyl 2 Oxovalerate, Tiglyl Carnitine, 2 Hydroxy 3 Methylvalerate, Isobutrylglycine, Methylsuccinate, Alpha Hydroxyisovalerate, Allo Isoleucine, Isobutyryulcarnitine, 3 Methylglutaconate, Tigloylglycine, N Acetlyisoleucine, 2 Methylbutyrylcarnitine (c5), Alpha Hydroxyisocaproate, N Acetylleucine, 3 Methyl 2 Oxobutyrate, 3 Hydroxy 2 Ethylpropionate, 4 Methyl 2 Oxopentanoate, 3 Hydroxyisobutyrate, Isoleucine, Beta Hydroxyisovalerate, Leucine, Isovalerylglycine, 5 Oxoproline, Cysteine Glutathione Disulfide, Cys Gly, Oxidized, Proline, N Acetylcitruline, Homoarginine, N2, n5 Diacetylornithine, Citrulline, Pro Hydroxy Pro, N Acetylarginine, Ornithine, Trans 4 Hydroxyproline, Dimethylarginine (sdma+Adma), Urea, Arginine, N Delta Acetylornithine, Homocitrulline, N Formylmethionine, Methionine Sulfone, S Methylcysteine, S Adenosylhomocysteine (sah), Cysteine, Cystine, N Acetylmethionine, N Acetyltaurine, 2 Aminobutyrate, Methionine, Cysteine S Sulfate, Alpha Ketobutyrate, 2 Hydroxybutyrate (ahb), Methionine Sulfoxide, 5 Hydroxyindoleacetate, Xanthurenate, C Glycosyltryptophan, Serotonin (5ht), N Acetylkynurenine (2), Indole 3 Carboxylic Acid, Indelopropionate, N Acetyltryptophan, Indolelactate, 3 Indoxyl Sulfate, Tryptophan, Kynurenate, Kynurenine, Tryptophan Betaine, 3 Methylhistidine, N Acetyl 1 Methylhistidine*, 1 Methylimidazoleacetate, N Acetyl 3 Methylhistidine*, Imidazole Lactate, 1 Methylhistidine, N Acetylthreonine, N Acetylserine, Dimethylglycine, Threonine, N Acetylglycine, Glycine, 3 Methylglutarylcarnitine (1), N6 Acetyllysine, Glutarylcarnitine (c5), Pipecolate, 3 Methylglutarylcarnitine (2), N2 Acetyllysine, N 6 Trimethyllysine, 2 Aminoadipate, Glutarate (pentanedioate), Lysine, 3 (3 Hydroxylphenyl) propionate, Tyramine O Sulfate, 4 Hydroxyphenylpyruvate, 3 (4)Hydroxyphenyl) lactate, Phenylpyruvate, Gentisate, P Cresol Glucuronide*. Phenylalanine, Vanilylmandelate (vma), 2 Hydroxyphenylacetate, Dopamine Sulfate (1), N Acetyltyrosine, Phenylacetylcarnitine, 3 Phenylpropionate (hydroccinnamate), Thyroxine, 3 Methoxytyrosine, Phenylacetylglutamine, P Cresol Sulfate, O Cresol Sulfate, Tyrosine, Phenyllactate (pla), N Acetylphenylalanine, 5 Methylthioadenosine (mta), 4 Acetamidobutanoate, Acisoga, Glutamine, Glutamate, N Acetylglutamine, Pyroglutamine*, N Acetylglutamate, 4 Guanidinobutanoate, Guanidinosuccinate, Creatine, Guanidinoacetate, Creatinine, N1 Methyl 2 Pyridone 5 Carboxamide, 1 Methylnicotinamide, Nicotinamide, Quinolinate, Trigonelline (n* Methylnicotinate), Riboflavin (vitamin B2), Bilirubin (e,e)*, Biliverdin, Bilirubin (z,z), Heme, I Uroblinogen, L Urobilin, Alpha Tocopherol, Gamma Tocopherol, Gamma Cehc Glucorinide*, Alpha Cehc Glucuronide*, Alpha Cehc Sulfate, Threonate, Gulonic Acid*, Oxalate (ethanedioate), Pantothenate, Pyridoxate, Hwesasllr, Bradykinin, Bradykinin Hydroxy Pro (3), Bradykinin Des Arg (9), Isoleucylglycine, Prolylglycine, Leucylglycine, Glycylleucine, Valylleucine, Gamma Glutamylthreonine*, Gamma Glutamyltryptophan, Gamma Glutamylmethionine, Gamma Glutamyltyrosine, Gamma Glutamylleucine, Gamma Glutamylglutamate, Gamma Glutamylvaline, Gamma Glutamylglutamine, Gamma Glutamylphenylalanine, Gamma Glutamylhistidine, Gamma Glutamylalanine, Gamma Glutamylisoleucine*, Gamma Glutamyl 2 Aminobutyrate, Adsgegdfxaefffvr*, Dsgegdtxaegggvr*, N Acetylcamosine, 2 Hydroxyisobutyrate, Ethyl Glucuronide, 2 Aminophenol Sulfate, 1,2,3 Benzenetriol Sulfate (2), O Sulfo L Tyrosine, Sulfate*, N Methylpipecolate, 3 Hydroxypyridine Sulfate, 2 Pyrrolidinone, Phenylcarnitine*, Indolin 2 One, Gluconate, Pipenne, Cihnamoylglycine, 1,6-Anhydroglucose, S′ Allylcysteine, Tartaqrate, Daldzein Sulfate (2), N 1, 2 Furoylglycine, N Acetylallin, Methyl Indole 3 Acetate, Betonicine, Piperidinone, Quinate, 2 Oxindole 3 Acetate, Dihydroferulic Acid, Ergothioneine, Erythotol, Allin, 2,3 Dihydroxyisovalerate, Methyl Glucopyranoside (alpha+Beta), 3 methyl Catechol Sulfate (1), 3 Hydroxyhippurate, 4 Ethylphenylsulfate, Catechol Sulfate, 4 Vinylphenol Sulfate, 3 Methyl Catechol Sulfate (2), Hippurate, O Methylcatechol Sulfate, Benzoate, 4 Hydroxycoumarin, 2 Acetamidophenol Sulfate, 7 Methylxanthine, 1 Methylxanthine, 1,7 Dimethylurate, Theobromine, 3 Methylxanthine, 1 Methylurate, 1,3 Dimethylurate, Caffeine, 5 ACetylamino 6 Formylamine 3 Methyluracil, 1,3.7 Trimethylurate, 7 Methylurate, Paraxanthine, 2,7 Dimethyurate, Tartronate (hydroxymalonage), Palmitoyl Sphingorryelin, Springosine, Palmitoleoyl Sphingomyelin, Sphinganine, Euricoyl Sphingomyelin, Sphingosine 1 Phosphate, Stearoyl Sphingomyelin, Nervonoyl Sphingomyelin*, Eicosenoyl Sphingomyelin*, Myristoyl Sphingomyelin*, Oleoyl Sphingomyelin, 3 Hydroxylaurate, 3 Hydroxyoctanoate, Alpha Hydroxycaproate, 2 Hydroxydecanoate, 2 Hydroxypalmitate, 2 Hydroxystearate, 5 Hydroxyhexanoate, 3 Hydroxydecanoate, 3 Hydroxysebacate, Pregnanediol 3 Glucuronide, 4 Androsetn 3 beta 1,7 beta diol disulfate, Epiandrosterone Sulfate, 5 alpha Pregnan 3 beta 20 alpha diol sulfate, 21 hydroxypregnenolone Disulfate, 4 Androsten 3 alpha 17 alpha diol monosulfate (3), 5 alpha androsten 3 beta 17 alpha diol disulfate, Dehydroisoandrosterone sulfate (hdea S), Corticosterone, 5 Pregnen 3b, 17 diol 20 One 3 sulfate, 4 Androsten 3 beta, 17 beta diol monosulfate (1), Etiocholanolone Glucuronide, TI18:1n7 (avaccenic Acid), TIdm 18:0 (plasmalogen Stearic Acid), TI20:3n6 (di Homo G Linoleic Acid), TI22:4n6 (adrenic acid), TI18:1n9 (oleic acid), TI20:3n9 (mead acid), TI16:1n7 (palmitoleic acid), TI22:5n6 (osbond acid), TI22:5n3 (docosapentaenoic acid), TI18:0 (stearic acid), TI24:1n9 (nervonic acid), TI20:0 (arachidic acid), TI14:0 (myristic acid), TI22:6n3 (docosahexaenoic acid), TI18:2n6 (linoleic acid), TIdm 18:1n6 (plasmalogen vaccenic acid), TI20:4n3 (eicosatetranoic acid), TI18:3n3 (a Linolenic Acid), TI22:1n9 (erucic acid), TI16:0 (palmitic Acid), TI22:0 (behenic acid), TI20:1n9 (eicosaenoic acid), TI14:1n5 (myristoleic acid), TI18:3n6 (g Linolenic Acid), TI20:2n6 (eicosadienoic acid), TI15:0 (pentadecanoic acid), TI20:5n3 (eicosdpentaenoic acid), TIdm 16:0 (plasmalogen palmitic acid), TI20:4n6 (arachidonic acid), 1 Linoleoylglycerophosphoethanolamine*, Stearoyl Linoleoyl Glycerophosphocholine (2)*, Palmitoyl Linoleoyl Glycerophosphocholine (2)*, 1 Arachidonoylglycerophosphoethanolaimne*, Palmitoyl Oleoyl Glycerophosphoglycerol (2), Palmitoyl Linoleoyl Glycerophosphoinositol (1), Stearoyl Arachidonoyl Glycerophodphoinositol (1)*, 2 Palmitoyltlycerophosphocholine*, 1 Linoleoylglycerophosphoinositol, 1 Palmitoylglycerophosphoethanolamine, 1 Stearoylglycerophosphoinositol, 1 Oleoylglycerophosphoinositol*, Palmitoyl Linoleoyl Glycerphosphocholine (1)*, Stearoyl Arachidonoyl Clycerophosphotehanolamine (1)*, 1 Oleoylplasmenylethanolamine*, 1 Palmitoleolglycerophosphocholine (16:1)*, 2 Stearolylglycerophosphocholine*, 1 Arachidonoylglycerophosphocholine (20:4n6)*, Palmitoyl Arachidonoyl Clycerophosphocholine (2)*, 2 Palmitoyleoylglycerophosphocholine*, 1 Arachidonoylglycerophosphoinositol, 1 Aachidonoylglycerphosophate, 1 Palmitoylglycerophosphocholine (16:0), Palmitoyl Arachidonoyl Glycerophosphocholine (2)*, Oleoyl Linoleoyl Glycerophosphoinositol (1)*, 1 Oleoylglycerophosphocholine (18:1), Stearoyl ARachidonoyl Glycerophosphocholine (2)*, 1 Oleoylglycerophosphoethanolamine, 1 Stearoylplasmenylethanolamine*, 1 Linoleoylglycerophosphocholine (18:2n6), 2 Stearoylglycerophosphoethanolamine*, Palmitoyl Oleoyl Glycerophosphocholine (1)*, 1 Palmitoylplasmenylethanolamine*, 1 Linoeoylglycerophosphocholine (18:3n3)*, 2 Stearoylglycerophosphoethanolamine*, Palmitoyl Oleoyl Glycerophosphocholine (1)*, 1 Palmitoylplasmenylethanolmaine*, 1 Linoleroylglycerophosphocholine (18:3n3)*, Stearoyl Linoleoyl Glycerophosphoethanolamine (1), Palmitoyl Palmitoyl Glycerophosphocholine(1)*, Stearoyl Linoleoy7l Glycerophosphocholine (1) *, 1 Stearolyglycerophosphocholine (18:0), 3 Hycroxybutyrate (bhba), Acetoacetate, Propionylglycine, Proionylcarnitine, Butyrylcarnitine, 10 Heptadecenoate (17:1n7), Nonadecanoate (19:0), Stearate (18:0), Myristate (14:0), Myristgleate (14:1n5), Margarate (17:0), Pentadecanoate (15:0), Palmitate (16:0), Arachidate (20:0), Eicosenoate (20:1n9 Or 11), Palmitoleate (16:1n7), 10 Nonadecenoate (19:1n9), Erucate (22:1n9), Trimethylamine N Oxide, Choline Phosphate, Glycerophosphorylcholine (gpc), Phosphotehanolamine, Glycerophosphoethanolamine, Hydroxybutyrylcarnitine, Octanoylcarnitine, Decanoylcarnitine, Myristoylcarnitine, Acetylcarnitine, Linoleoylcarnitine, Stearoylcarnitine, Oleoylcarnitine, Laurylcarnitine, Cis 4 Decenoyl carnitine, Dihomo Linolenate (20:3n3 Or N6), Arachidonate (20:4n6), Stearidonate (18:4n3), Docosahexaenoage (dha:22:6n3), Linolenate alpha Or Gamma: (18:3h3 Or6), Adrenate (22:4n6), Docosapentaenoate (n3 Dpa: 22 5n3), Eicosapentaenoate (epa: 20:5n3), 1 Linoleoylglycerol (1 Monolinolein), 1 Linolenoylglycerol, 1 Palmitoylglycerol (1 Monopalmitin), 2 Oleoylglycerol (2 Monoolein), 1 Stearoylglycerol (1 Monostearin), 1 Oleoylglycerol (1 Monoolein), 1 Docosahexaenoylglycerol, 1 Dihomo Linolenylglycerol (alpha, Gamma), 1 Arachidonylglycerol, Maleate (cis Butenedioate), 3 Methyladipate, Dodecanedioate, Eicosanodioate, Hexadecanedioate, 3 Carboxy 4 Methyl 5 Propyl 2 Furanpropanoate (cmpl), Tetradecanedioate, Octadecanedioate, Linoleamide (18:2n6), Palmitic Amide, Oleamide, Taurodeoxycholate, Deoxycholate, Ursodeoxycholate, Taurocholenate Sulfate, Taurolithocholate 3 Sulfate, Glycocholenate Sulfate*, Taurocholate, Cholate, Tauro Beta Muricholage, Oleic Ethanolamide, N Oleoyltaurine, Palmitoyl Ethanolamide, 7 Alpha Hydroxy 3 Oxo 4 Cholestenoate (7 Hoca), Cholesterol, Laurate (12:0), Caprate (10:0), Caprylate (8:0), 5 Dodecenoage (12:1n7), TItI (total Total lipid), Myo Inositol, Chiro Inositil, N Palmitoyl Glycine, Hexanoylglycine, N Linoleoylglycine, 2 Methylmalonyl Carnitine, Malonylcarnitne, Carnitine, Deoxycarnitine, 2 Aminooctanoate, 2 Aminoheptanoate, 3 Hydroxy 3 Methylglutarate, 13 Methylmyristic Acid, 15 Methylpalmitate (isobar With 2 Methylpalmitate), Beta Alanine, Pseudouridine, Uridine, Adenosine 5′ Monophosphate (amp), N1 Methyladenosine, N6 Succinyladenosine, N6 Carbamoylthreonyladenosine, Cytidine, N4 Acetylcytidine, Dihydroorotate, Orotidine, Hypoxanthine, Inosine, Allantoin, Xanthosine, Xanthine, Urate, N1 Methylguanosine, N2, n2 Dimethylguanosine, 1,5 Anhydroglucitol (1,5 Ag), Lactate, Glucose, Glycerate, Pyruvate, Xylitol, Arabitol, Threitol, N Acetylneuraminate, Glucuronate, Erythronate*, Mannitol, Mannose, Fumarate, Succinate, Succinylcarnitine, Malate, Alpha Ketoglutarate, Citrate, Phosphate.

Clusters of interrelated multi-omic measurements were identified across individuals using community analysis, an unsupervised approach that iteratively prunes the network to reveal densely interconnected subgraphs (communities) (Girvan and Newman, 2002). Seventy communities of at least two vertices (mean of 10.9 V and 34.9 E) were identified in the network at the cutoff with maximum community modularity (Newman, 2006), and are fully visualized as both a dendrogram and an interactive graph in Cytoscape (Shannon, et al., (2003). Genome Res. 13, 2498-2504). 70% of the edges in the correlation network remained after community edge pruning. The communities often represented a cluster of physiologically-related analytes, as described below.

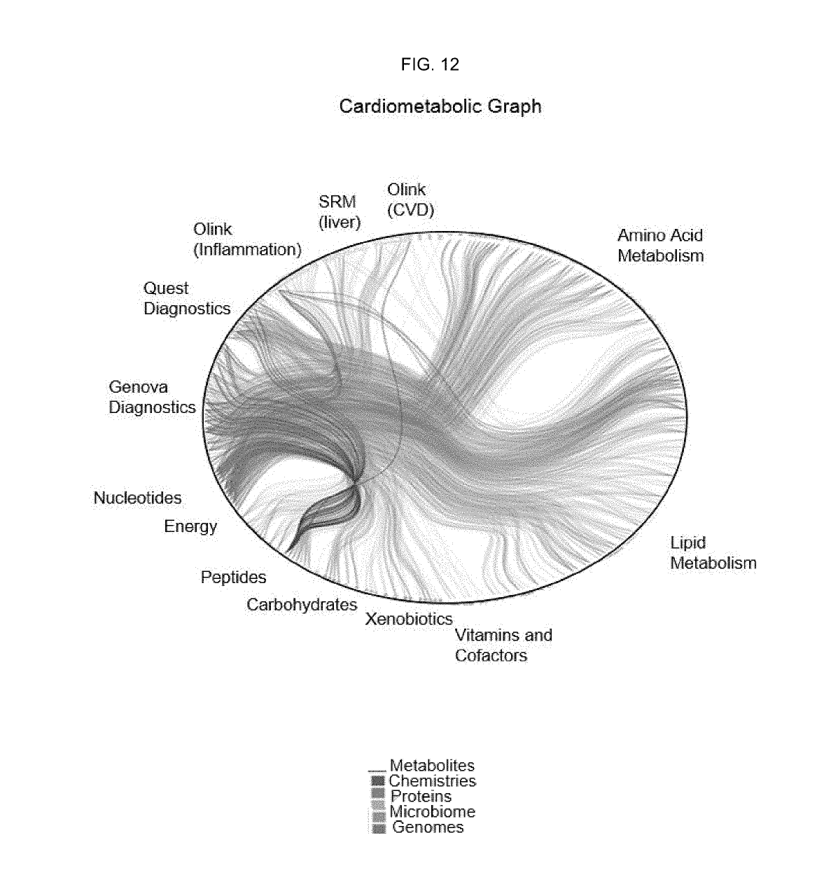

The largest community (246 V; 1645 E) contains many clinical analytes associated with cardiometabolic health, such as C-peptide, triglycerides, insulin, HOMA-IR, fasting glucose, HDL cholesterol, and small LDL particle number (FIG. 12). All vertices and edges of the cardiometabolic community, with lines indicating significant (p_{adj}<0.05) correlations are shown. Associations with FGF21 and gamma-glutamyltyrosine are highlighted (FGF21 correlations in dark grey lines originating in Olink (Inflammation) and extending into portions designated as Genova Diagnostics and Nucleotides; gamma-glutamyltyrosine correlations in dark grey lines originating in Peptides and extending into portions designated as Nucleotides; Genova Diagnostics; Quest Diagnostics and Olink (CVD). The four most connected clinical analytes by degree (i.e. the number of edges connecting a particular analyte) are C-peptide (99), insulin (88), HOMA-IR (88), and triglycerides (75). The four most connected proteins by degree are leptin (18), C-reactive protein (15), fibroblast growth factor 21 (FGF21) (14), and inhibin beta C chain (INHBC) (10). Leptin and c-reactive protein are indicators for cardiovascular risk (Koh, et al., (2008). Circulation 117, 3238-3249; Ridker (2003). Circulation 107, 363-369). FGF21 is positively correlated with the clinical analytes C-peptide (ρ=0.51; p_{adj}=3.1e-3), triglycerides (ρ=0.50; p_{adj}=3.3e-3), HOMA-IR (ρ=0.50; p_{adj}=3.6e-3), insulin (ρ=0.47; p_{adj}=9.0e-3), and small LDL particle number (ρ=0.42; p_{adj}=4.3e-3), and is an emerging biomarker for cardiometabolic disorders (Woo, et al., (2013). Clin. Endocrinol. (Oxf) 78, 489-496). INHBC, a member of the TGF-beta superfamily, is similarly positively correlated with the clinical analytes triglycerides (ρ=0.45; p_{adj}=3.0e-3), small LDL particle number (ρ=0.43; p_{adj}=6.8e-3), C-peptide (ρ=0.40; p_{adj}=1.8e-2), HOMA-IR (ρ=0.38; p_{adj}=3.4e-2), and insulin (ρ=0.38; p_{adj}=3.8e-3), but is currently uncharacterized as a marker for cardiovascular risk. Of interest, serum amyloid β component (SAP) was positively correlated with LDL particle number (ρ=0.39; p_{adj}=1.8e-3), but not LDL cholesterol. SAP is a universal constituent of amyloid deposits, including those observed in Alzheimer's disease (Duong, et al., (1989). 78, 429-437.), and is associated with myocardial infarction in older men (Jenny, et al., (2007). Arterioscler. Thromb. Vasc. Biol. 27, 352-358.).

The Metabolites included in the network of FIG. 12 can include one or more of Guanidinosuccinate, Alanine, Isovalerylcarnitine, Valine, N Acetylleucine, 2 Methylbutyrylcarnitine (c5), Isoleucine, Leucine, S Adenosylhomocysteine (sah), Cysteine, Cystine, Methionine Sulfone, N Acetyltryptophan, N Acetylkynurenine (2), 3 Indoxyl Sulfate, Xanthurenate, Kynurenine, Kynurenate, Tryptophan, Phenylalanine, N Acetylphenylalanine, 4 Hydroxyphenylpyruvate, Phenylpyruvate, Tyrosine, N Acetyltyrosine, Phenylacetylcarnitine, Glutamine, Glutamate, N Acetylglycine, Glycine, Proline, N Delta Acetylornithine, N Acetylcitrulline, Homoarginine, N2, n5 Diacetylornithine, Pro Hydroxy Pro, 2 Aminoadipate, Lysine, Deoxycholate, Ursodeoxycholate, Arachidate (20:0), Nonadecanoate (19:0). Palmitate (16:0), Erucate (22:1n9), TI16:0 (palmitic Acid), TI16:n7 (palmitoleic Acid), TI18:1n7 (avvaccenic Acid), TI14:1n5 (myristoleic Acid), TI24:1n9 (nervonic Acid), TIdm:18:1n7 (plasmalogen Vaccenic Acid), TIdm 18:0 (plasmalogen Stearic Acid), TI14:0 (myristic Acid), TI18:2n6 (linoleic Acid), TIdm16:0 (plasmalogen Palmitic Acid), TI22:1n9 (erucic Acid), TI20:3n6 (di Homo G Linoleic Acid), TI20:4n3 (eicosatetranoic Acid), TI18:1n9 (oleic Acid), TI18:3n3 (a Linolenic Acid), TIdm 18:1n9 (plasmalogen Oleic Acid), 1 Linoleoylglycerophosphocholine (18:2n6), 1 Linolenoylglycerophosphocholine (18:3n3)*, 2 Stearoylglycerophosphocholine*, 1 Palmitoleoylglycerophosphocholine (16:1)*, 1 Oleoylglycerophosphocholine (18:1), 3 Hydroxylaurate, 2 Hydroxydecanoate, 3 Hydroxydecanoate, 3 Hydorxyoctanoate, 2 Hydroxystearate, 3 Hydroxysebacate, 7 Alpha Hydroxy 3 Oxo 4 Cholestanoate (7 Hoca), Cholesterol, Carnitine, Pregnanediol 3 Glucuronide, Epiandrosterone Sulfate, Stearoylcarnitine, Myristoleoylcarnitine*, Decanoylcarnitine, Laurylcarnitine, 2 Oleoylglycerol (2 Monoolein), 1 Linolenoylglycerol, 1 Palmitoylglycerol (1 Monopalmitin), 1 Linoleoylglycerol (1 Monolinolein), 1 Dihomo Linolenylglycerol (alpha, Gamma), 1 Oleoylglycerol (1 Monoolein), Caprate (10:0), Laurate (12:0), Caprylate (8:0), 5 Dodecenoate (12:1n7), Palmitoyl Sphingomyelin, Stearoyl Sphingomyelin, Sphinganine, Nervonoyl Sphingomyelin*, Sphingosine, Oleoyl Sphingomyelin, 3 Hydroxybutyrate (bhba), Acetoacetate, Butyrylcarnitine, Propionylcarnitine, Dihomo Linolenate (20:3n3 or N6), Hexanoylglycine, Glycerophosphoethanolamine, TItl (total Total Lipid), Eicosanodioate, Octadecanedioate, 3 Methyladipate, 2 Methylmalonyl Carnitine, Palmitoyl Ethanolamide, N Oleoyltaurine, N1 Methyl 2 Pyridone 5 Carboxamide, Nicotinamide, Alpha Tocopherol, Gamma Tocopherol, Threonate, Oxalate (ethanedioate), Ergothioneine, N AcetylalliinErythritol, Cinnamoylglycine, S Allylcysteine, 2 Pyrrolidinone, 2 Hydroxyisobutyrate, Tartronate (hydroxymalonate), 1,3,7 Trimethylurate, 4 Hydroxycoumarin, 2 Acetamidophenol Sulfate, 4 Acetylphenol Sulfate, Mannose, Erythronate*, Pyruvate, Lactate, Glucose, Glycerate, Xylitol, Gamma Glutamylleucine, Gamma Glutamylphenylalanine, Gamma Glutamylisoleucine*, Gamma Glutamylglutamine, Gamma Glutamylhistidine, Gamma Glutamylglutamate, Gamma Glutamyltyrosine, Bradykinin, Hydroxy Pro(3), Glycylleucine, Succinylcarnitine, Succinate, Fumarate, Malate, Alpha Ketoglutarate, Citrate, Xanthine.

The analytes associated with the Chemistries included in the network of FIG. 12 can include one or more of Ferritin, LDL Small, LDL Particle Number, Glucose, Chloride, LDL Peak Size, Alkaline Phosphatase, LDL Pattern, LDL Medium, Bilirubin Direct, Triglycerides, GGT, HDL Large, Alanine, Tyrosine, Alpha Amino N Butyric Acid, Kyneurenic Quinolinic Ratio, Gondoic Acid, Glucose, Pyroglutamic Acid, Magnesium, Raio Gln Gln, FIGLU, Average Inflammation Score, Homovanillic Acid, hs-CRP, Homogentisic Acid, Succinic Acid, Lignoceric Acid, HOMA-IR, Ratio Asn Asp, Small LDL Particle, Interleukin IL6, Phenylalanine, Adiponectin, Indoleacetic Acid, HDL Cholesterol, C-Peptide, Quinolinic Acid, Isovalerylglycine, Linoleic Dihomo Gamma Linoleic, Lactic Acid, Weight, 5 Hydroxyindoeacetic Acid, Vitamin D, Lysine, Tryptophan, Total LC Omega 9, Body Mass Index, Leptin, Glutamic Acid, Dihomo Gamma Linolenic Acid, Manganese, Triglycerides, Gamma Linolenic Acid, Insulin, Hba1C, Proinsulin, hs-CRP Relative Risk, LDL Particle.

The dynamic analytes associated with the Chemistries of FIG. 11 can include one or more of Total Saturated, Taurine, Margaric Acid, Coenzyme Q10 Ubiquinone, Ethanolamine, Citramalic Acid, Hs Crp, Vanilmandelic acid, Citric Acid, Body Mass Index, Tryptophan, Eicosapentaenoic Acid, Total Omega 6, Hs Crp Relative Risk, Vitamin D, 5 Hydroxyindoeacetic acid, Pai 1 Relative Risk, Oleic Acid, Suberic Acid, Beta Aminoisobutyric Acid, Glutamine, Lead, Figlu, Indoleacetic Acid, Palmitic Acid, Alpha Ketoglutaric Acid, Rbc Sample Weight, Arachidic Acid, Succinic Acid, Glucose, Tyrosine, Abarinose, Isovalerylglycine, Medication Cholesterol, Methylglutaric Acid, Xanthutenic Acid, Phosphoethanolamine, Average Inflammation Score, Gondoic Acid, Gamma Linolenic Acid, Interleukin 116, Interleukin 118, Pentadecanoic Acid, Ldl Cholesterol, Ornithine, Copper, Beta Hydroxybutyric Acid, Orotic Acid, Urine Creatinine Organic Acids, 2 Hydroxyphenylacetic Acid, Rba1C, Insulin, Small Ldl Particle, Hippuric acid, Ratio Gln Gln, Glutamic Acid, Cysteine, Glycine, Alpha Ketophenylacetic Acid, Alpha Aminoadipic Acid, Arachidonic Acid, Lactic Acid, Total Cholesterol, 3 Hydroxyisovaleric Acid, Isoleucine, Beta Alanine, Pyruvic acid, Aketoisovaleric Acid, Arachidonic Eicosapentaenoic, Selenium, Histidine, Tricosanoic Acid, Ratio Asn Asp, 3 Hydroxypropionic Acid, Omega 3 Index, Serine, Tartaric Acid, Docosapentaenoic Acid, Threonine, Adipic Acid, Hdl Particle, Behenic Acid, R Onda, Ratio Om6 Om3, Alpha Ketoadipic Acid, Nervonic Acid, Lysine, Total Omega 3, Isocitric Acid, Triglycerides, Pari, Height, Arginine, Leptin, Proline, Tin, Cis Aconitic Acid, Elaidic Acid, Ldl Particle, homogentisic acid, Infalpha relative risk, 3 Hydroxyphenylacetic acid, Methionine, Citrulline, Leucine, Mercury, 3 Methylhistidine, Interleukin 118 Relative Risk, Kynurenic Quinolinic Ratio, Methylmalonic acid, alpha amino n butyric acid, sarcosine, alanine, cysteine, gamma aminobutyric acid, proinsulin, urea, Rep Index, Matic Acid, Vaccenic Acid, Manganese, Dihomo Gamma Linolenic Acid, Ammonia, Benzoic Acid, Aspartic Acid, Phenylacetic Acid, Valine, Total Lc Omega 9, Glutaric Acid, A Keto B Methylvaleric Acid, Gluathione, Weight, Adiponectin, Linoleic Dihomo Gamma Linoleic, 3 Methyl 4 Hydroxyphenylglycol, Quinolithic Acid, Asparagine, A Ketoisocaproic Acid, Phenylalanine, C Peptide, Pyroglutamic Acid, Lignoceric Acid, Docosatetraenoic Acid, Phosphoserine, Stearic Acid, Homa Ir, Homovanillic Acid, Eicosadienoic Acid, Magnesium, Ldl Medium, Globulin, Ldl Particle number, Egfr African American, Ferritin, Phosphate, Homocysteine, Carbon Dioxide, Calcium, Hdl Large, Cholesterol Total, Ldl Pattern Alt, Urea Nitrogen, Methylmalonic Acid, Ast, Chloride, Bilirubin Direct, Alkaline Phosphatase, Albumin, Glucose, Ggt, Protein Total, Triglycerides, Egfr Non African American, Creatinine, Uric Acid, Sodium, Ldl Peak Size, Albumin Globulin ratio, Ldl Small, Bilirubin Total, Ld.

The Proteins included in the network of FIG. 11 can include one or more of ACTA2, PTGO2, ACTA2(2), PPBP, PPBP(s), NCF2, F9, SERPINC1, INHBC, APCS, GC, HGFAC, MBL2, CFHR1, CRP(s), MBL2(2), CBP, VIN, F9(2), OSM, MCP 4, IL8, IL2, IL6, MORA, FGF21, SIRT2, VEGF A, STAMPB, FLi3LI, TNFSF14, IL 18R1, CSCL10, IL20RA, CCL9, DNER, CO5, CCL50, CCL23, CSF1, OPG, CD40, 4EBP1, AXIN1, HGF, BEN, TPA, CSTB, MMP10, SIRT2, VEGE D, VEGE A, PDGF SUBUNIT B, LEP, BETA NGE, EGF, IL1Ra FABP4, GH, HSP 27, CD40, NEMO.

The dynamic analytes associated with the Microbiome included in the network of FIG. 11 can include one or more of Diversity, Pasteurellales, Coriobacteriales, Verrucomicrobiales, Coriobacteriaceae, 91 otu 13421, Verrucomicrobiaceae, Desulfovibrionaceae, Pasteurellaceae, 91 otu 1825, 91 otu4418, Unclassified, Christensenellaceae, Peptostreptococcaceae, Mogibacteriaceae, Coriobacterila, verrucomicrobiae, deltaproteobacteria, mollicutes, tenericutes, verrucomicrobia.

The dynamic analytes associated with the Genomes of the network of FIG. 11 can include one or more of Bilirubin Levels, Allergic Sensitization, Inflammatory Bowel Disease, Activated Partial Thromboplastin Time, Deletion Cfhr1, Bladder Cancer, Plasma Omega 6 Polyunsaturated Fatty Acid Levels (arachidonic Acid), Plasma Omega 6 Polyunsaturated Fatty Acid Levels (adrenic acid), Plasma Omega 6 Polyunsaturated Patty Acid Levels (gamma Linolenic Acid), Plasma Omega 6 Polyunsaturated Fatty Acid Levels (Linoleic Acid), Omega 6 Polyunsaturated Fatty Acid Levels (dihomo Gamma Linolenic Acid).

The Proteins included in the network of FIG. 12 can include one or more of IL 1RA, CSTB, FABP4, IL 6, VEGF D, VEGF A, LEP, T PA, SERPINC1, F9, GC, MBL2, MBL2(2), CRP(2), INHBC, APCS, F9(2), CRP, OSM, IL 18R1, VEGF A, CD40, CCL20, TNFSF14, CXCL 10, IL 6, IL 10RA, FGF21, HGF, CCL 19.

The dynamic analytes associated with the Genomes included in the network of FIG. 12 can include Omega 6 Fatty Acid Levels (DGLA) and the dynamic analytes associated with the Microbiomes included in the network of FIG. 10 can include one or more of Pasteurellaceae, Pasteurellales.

One of the most interconnected metabolites in the cardiometabolic community is gamma-glutamyltyrosine (27), which is significantly correlated with markers of metabolic syndrome (e.g. glucose (ρ=0.41; p_{adj}=1.6e-3), HOMA-IR (ρ=0.38; p_{adj}=6.0e-3), and insulin (ρ=0.36; p_{adj}=9.7e-3)), as well as cardiovascular risk (triglycerides (ρ=0.41; p_{adj}=1.5e-3), small LDL particle number (ρ=0.35; p_{adj}=1.5e-2), and HDL cholesterol (ρ=−0.35; p_{adj}=1.6e-2)). Gamma-glutamyltyrosine is a product of the action of the enzyme gamma-glutamyl transferase (GGT), which is a known biomarker of diabetes risk (Bradley, et al., (2013). Biomark Med 7, 709-721.; Lim, et al., (2007). Clin. Chem. 53, 1092-1098). An ordinary least squares (OLS) regression was performed with homeostatic risk assessment (HOMA-IR, a common marker for insulin resistance), as the dependent variable and GGT, gamma-glutamyltyrosine, age, sex, and BMI as the regressors (R²_{adj}=0.46) (Table 6. In this model, gamma-glutamyltyrosine has a more significant effect on HOMA-IR (p<0.001) than does GGT (p=0.09), potentially representing a superior biomarker candidate.

TABLE 6
OLS regression on the dependent variable HOMA-IR. Regressors include
sex, GGT, gamma-glutamyltyrosine, age, and body mass index. Body
mass index and gamma-glutamyltyrosine are significant regressors
in the model, while GGT is marginally significant.

| | | std | | | 95% | |
|---|---|---|---|---|---|---|
| regressor | coefficient | error | t | p-value | confidence | |
| Body Mass Index | 1.5423 | 0.318 | 4.844 | <0.001 | 0.911 | 2.174 |
| gamma- | 1.2984 | 0.267 | 4.859 | <0.001 | 0.768 | 1.828 |
| glutamyltyrosine | | | | | | |
| GGT | 0.1233 | 0.073 | 1.696 | 0.093 | −0.021 | 0.267 |
| Age | −0.2744 | 0.22 | −1.247 | 0.215 | −0.711 | 0.162 |
| Sex [Male] | −0.0114 | 0.01 | −1.169 | 0.245 | −0.031 | 0.008 |

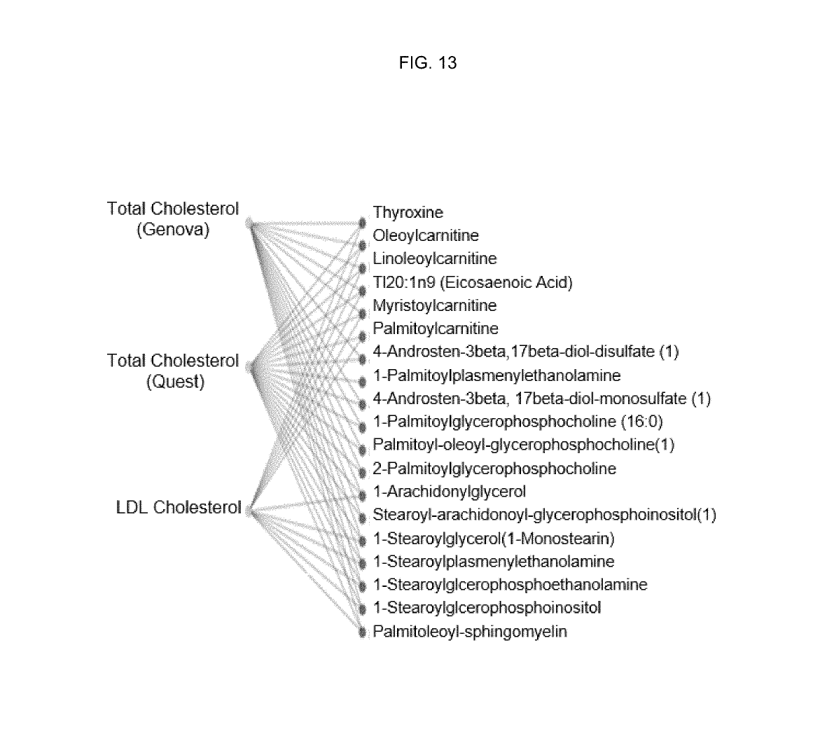

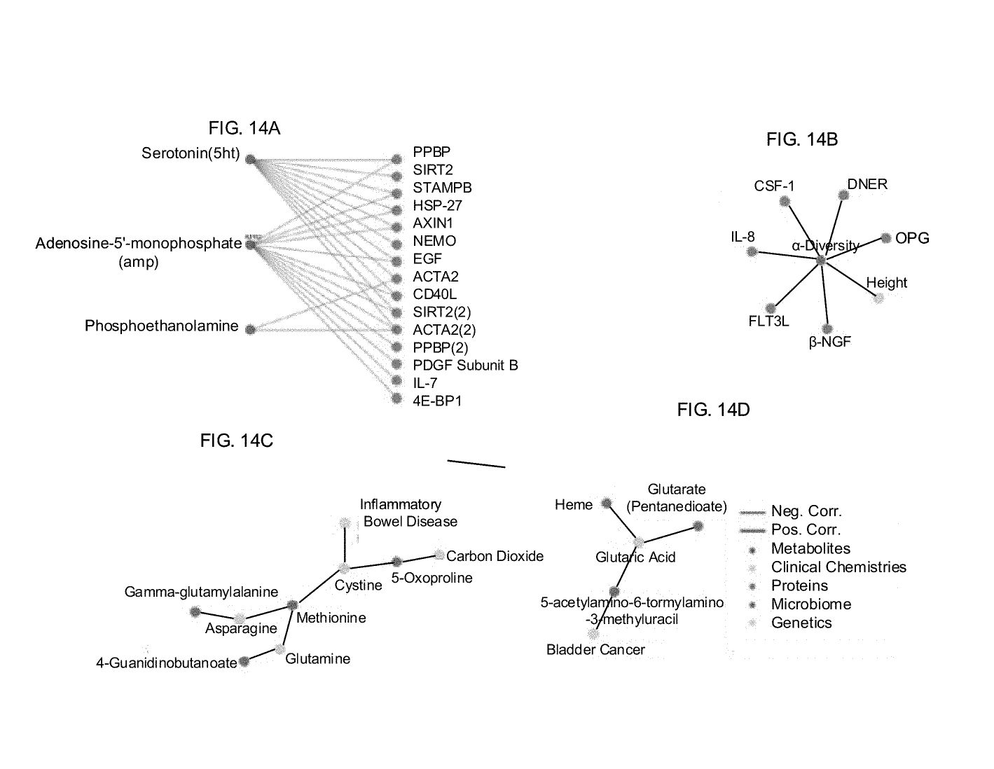

FIGS. 13 and 14A-14D show correlations between additional communities: (13) Serotonin community (14A) Cholesterol community (14B) α-diversity community (14C) The genetic risk for inflammatory bowel disease is negatively correlated with cystine (14D) The genetic risk for bladder cancer is positively correlated with 5-acetylamino-6-formylamino-3-methyluracil (AFMU).

Total cholesterol and LDL cholesterol (LDL-C) segregate into a separate community from the cardiometabolic community (22 V; 48 E) with a broad array of plasma lipids (FIG. 13). Thyroid hormone L-thyroxine is also present and is negatively correlated with total cholesterol levels (ρ=−0.44; p_{adj}=5.0e-4) as well as LDL cholesterol (ρ=−0.41; p_{adj}=2.1e-3). Hypothyroidism has long been recognized clinically as a cause of elevated cholesterol values (Althaus, et al., (1988). Clin. Endocrinol. (Oxf) 28, 157-163.).

A community formed around plasma serotonin (18 V; 25 E) containing twelve proteins listed in Table 7 for which the most significant enrichment identified in a STRING gene ontology analysis (Jensen, et al., (2009). Nucleic Acids Res. 37, D412-D416.) was platelet activation (p_{adj}=1.7e-3) (FIG. 14B). Serotonin is known to induce platelet aggregation (Li, et al., (1997). Blood Coagul. Fibrinolysis 8, 517-523); accordingly, selective serotonin reuptake inhibitors (SSRIs) may protect against myocardial infarction (Sauer, et al., (2001). Circulation 104, 1894-1898).

TABLE 7
Proteins present in the serotonin community

| Symbol | Name | uniprot |
|---|---|---|
| ACTA2 | actin, alpha 2, smooth muscle, aorta | P62736 |
| EGF | epidermal growth factor | P01133 |
| SIRT2 | sirtuin 2 | Q8IXJ6 |
| PPBP | pro-platelet basic protein | P02775 |
| CD40LG | CD40 ligand | P29965 |
| AXIN1 | axin 1 | O15169 |
| HSPB1 | heat shock 27 kDa protein 1 | P04792 |
| STAMBP | STAM binding protein | O95630 |
| EIF4EBP1 | eukaryotic translation initiation factor 4E binding | Q13541 |
| | protein 1 | |
| PDGFB | platelet-derived growth factor beta polypeptide | P01127 |
| IL7 | interleukin 7 | P13232 |
| IKBKG | inhibitor of kappa light polypeptide gene enhancer | P62736 |
| | in B-cells, kinase gamma | |

Several communities containing microbiome taxa were observed, suggesting specific microbiome-analyte relationships. Hydrocinnamate, L-urobilin, and 5-hydroxyhexanoate clustered with the bacterial class Mollicutes and family Christensenellaceae (8 V; 8 E). Another community emerged around families Verrucomicrobiaceae and Desulfovibrionaceae and p-cresol-sulfate (7 V, 6 E). The families Coriobacteriaceae and Mogibacteriaceae are associated (12 V, 19 E) with levels of phenylacetic acid, eicosadienoic acid, p-cresol-glucuronide, taurine, and phenylacetylglutamine. Phenylacetylglutamine, a known microbial metabolite (Li et al., 2008), has recently been identified as a risk factor for mortality and cardiovascular disease in chronic kidney disease patients (Poesen et al., 2016). Finally, the bile acid cholate clusters with the family Peptostreptococcaceae (2 V, 1 E).

A community formed around microbiome α-diversity (8 V, 7 E), a measure of the number of operational taxonomic units (OTUs) observed as well as the evenness of their distributions, where elevated diversity is generally thought to be associated with greater health in part by ameliorating inflammation (Manichanh, et al., (2006). Gut 55, 205-211). Microbiome α-diversity was negatively correlated with inflammatory and immune-related proteins, including interleukin-8 (IL-8), FMS-related tyrosine kinase 3 (FLT3LG), and macrophage colony-stimulating factor 1 (CSF1) (FIG. 12). In contrast, β-nerve growth factor (NGF) had a positive relationship with microbiome α-diversity. An analysis with STRING (Jensen et al., 2009) on α-diversity community members revealed a significant enrichment in the KEGG pathway cytokine-cytokine receptor interaction (p_{adj}=1.1e-4), of which other pathway members have been observed to be implicated in the pathogenesis of inflammatory bowel disease (Jostins, et al., (2012). Nature 491, 119-124.).

Using data from genome-wide association studies (GWAS), each participant's set of polygenic scores was calculated for specific diseases and quantitative traits, and those scores were correlated with the measured blood analytes. Calculation began with a targeted evaluation of the polygenic score associated with LDL cholesterol (LDL-C) (Global Lipids Genetics Consortium, Willer, et al., (2013). Nat. Genet. 45, 1274-1283.). The hypothesis that the cumulative effect of these 59 common variants would correlate with LDL-C levels, even in the presence of powerful environmental influences such as diet and exercise was tested.

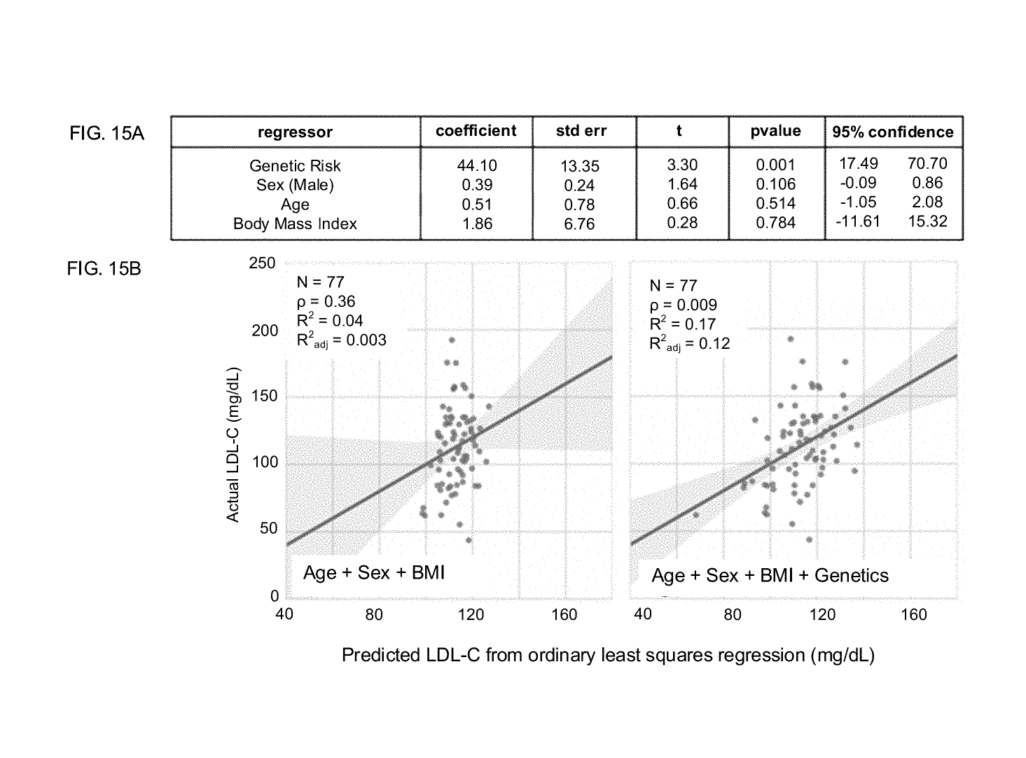

FIGS. 15A, 15B show cumulative genetic risk being predictive of LDL-C levels: (15A) OLS regression on the dependent variable LDL-C, (15B) Spearman correlations between actual LDL-C and predicted LDL-C based on three OLS models, while excluding participants on cholesterol-lowering medication (N=77). Spearman's ρ between predicted and actual LDL-C is also shown. An OLS regression was performed on a number of potential predictors for levels of LDL-C, including age, sex, body mass index (BMI) and the cumulative genetic risk score described above. Combining age, sex, and BMI alone in the regression model did not yield a significant prediction of the levels of LDL-C(R²=0.04; R²_{adj}=0.003; p=0.36). When genetic risk was included in the model it became significantly predictive of actual LDL-C levels, even when adjusting the fit to control for the additional variable (R²=0.10; R²_{adj}=0.07; p=0.03). Excluding individuals on cholesterol-lowering medications further improved its accuracy (R²=0.17; R²_{adj}=0.12; p=0.009) (FIGS. 15A, 15B). To estimate the robustness of the model, 10-fold cross validation was performed. R² did not decrease substantially, suggesting overfitting was not a dominant factor in the original model (R²=0.11; R²_{adj}=0.09).

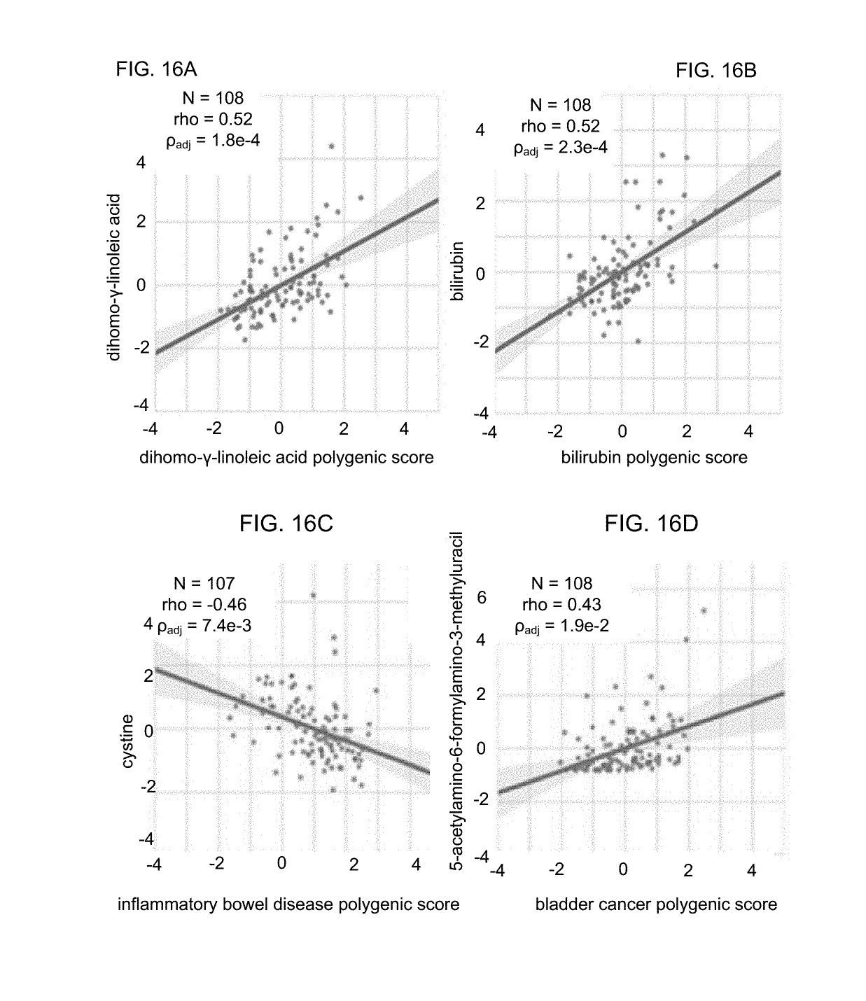

Polygenic risk scores correlate with analytes associated with condition risk. Several edges in the network were genetic traits correlated with their corresponding biomarker from previously published studies. For example, the observed blood levels of dihomo-γ-linolenic acid (DGLA) were strongly correlated (ρ=0.52; p_{adj}=1.8e-4) with the risk computed from genotypes in six variants previously associated with DGLA levels (Guan, et al., (2014). Circ Cardiovasc Genet 7, 321-331.) (FIG. 16A). Similar results were observed for other omega-6 fatty acids including arachidonic acid, linoleic acid, and eicosadienoic acid as well as bilirubin, a marker of liver dysfunction (ρ=0.52; p_{adj}=2.3e-4) (Dai, et al., (2013). Genet. Epidemiol. 37, 293-300.) (FIG. 16B).

While GWAS that model measurable analytes are the most directly applicable to the measurements in the study, other edges in the network appeared between polygenic disease risk and specific analytes. For example, the genetic risk of inflammatory bowel disease (IBD) in Europeans has been associated with 110 SNVs (Jostins et al., 2012). In the cohort, the polygenic risk score for IBD calculated from these variants was significantly negatively correlated with the plasma concentration of cystine, the disulfide form of cysteine (ρ=−0.46; p_{adj}=7.4e-3) (FIGS. 14C and 16C).

A bladder cancer genetic risk score for all of the participants was computed from nine SNVs previously associated with bladder cancer in a European cohort (Rothman et al., 2010). An edge was identified between this polygenic risk score for bladder cancer and levels of 5-acetylamino-6-formylamino-3-methyluracil (AFMU), an acetylated metabolite of caffeine, in the cohort (ρ=0.43; p_{adj}=1.9e-2). One of the variants is located downstream of the gene NAT2 for the enzyme N-acetyltransferase 2 responsible for acetylating carcinogenic compounds in urine. Polymorphisms in NAT2 are known to produce ‘fast’ and ‘slow’ acetylator phenotypes, of which the latter convey particular risk for bladder cancer (Okkels et al., 1997) (FIGS. 14D and 16D).

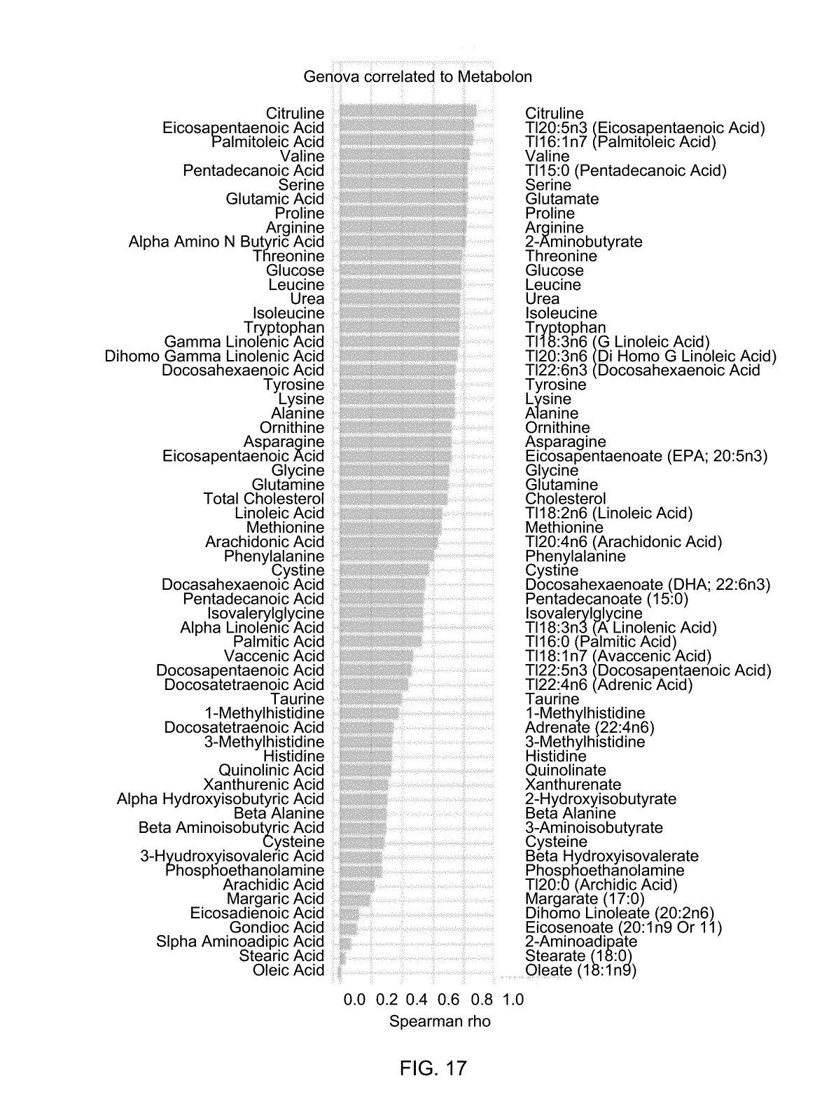

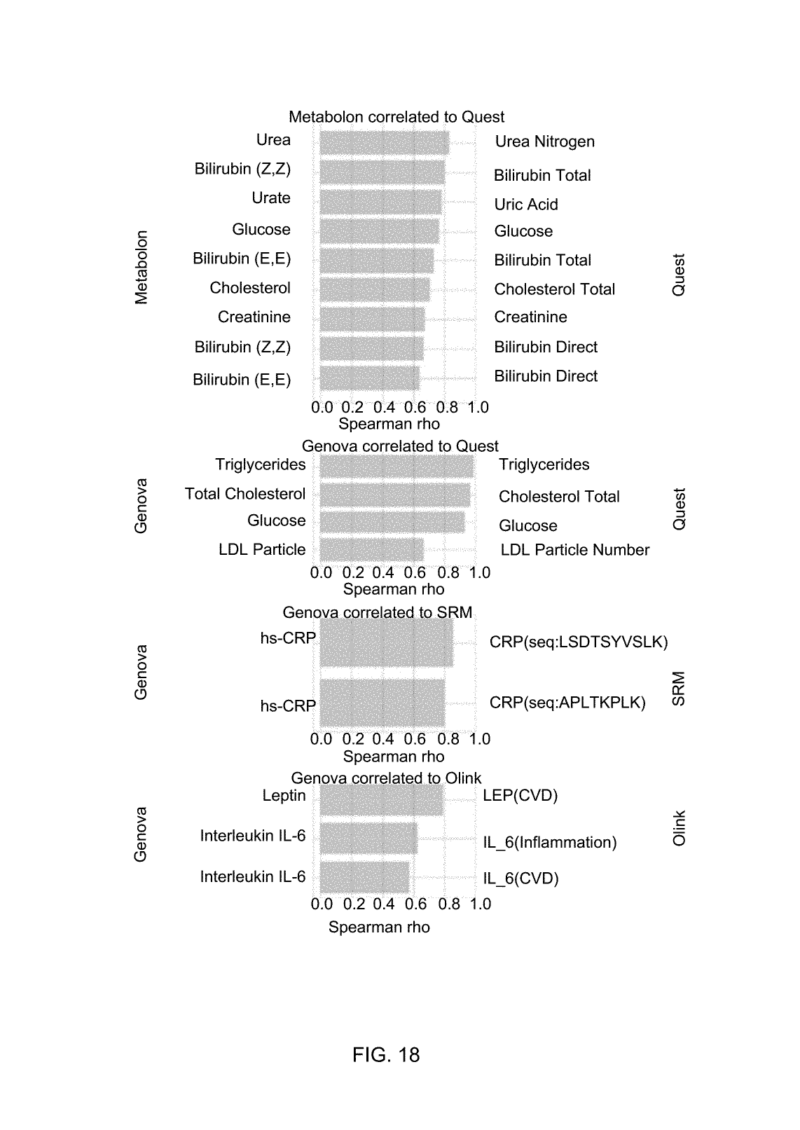

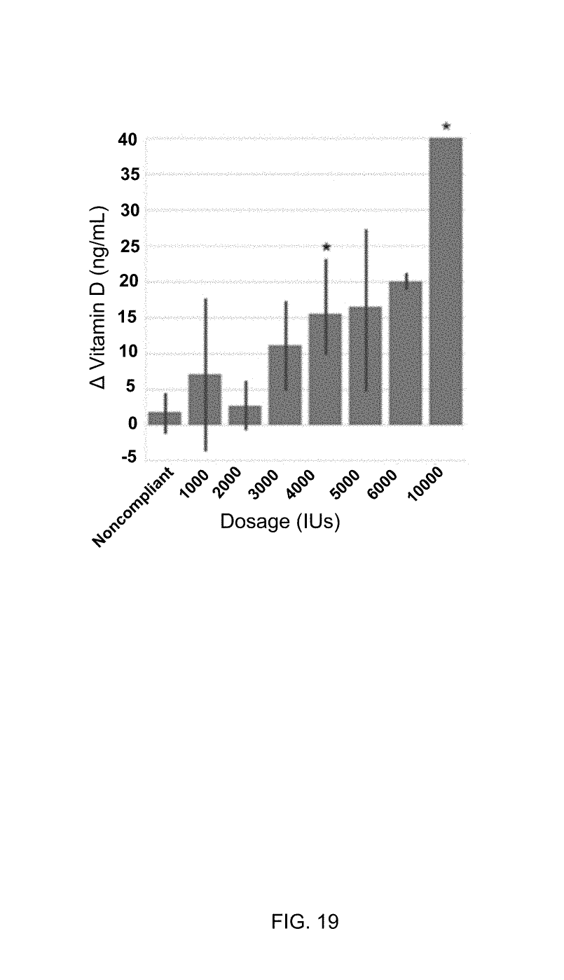

Supplemental Results: Technical Reproducibility. Most clinical laboratory measurements were assayed by only one of the vendors (Quest or Genova) but certain measurements were measured by both due to overlaps in the standard analysis panels. Additionally, some analytes from the metabolomics and proteomics were also measured by the clinical labs. As a result, comparison of these analytes could be performed, as shown in FIGS. 17 and 18. FIGS. 17 and 18 show reproducibility across different vendors. Several proteins and metabolites were measured by multiple vendors. Shown in FIGS. 17 and 18 are the Spearman correlations between these repeated measurements, sorted in descending order by rho. Triglycerides, total cholesterol, and fasting glucose show high levels of concordance, LDL particle number exhibits greater variation between the two labs. Glucose from the metabolomics data correlates with Quest (ρ=0.80) and Genova (ρ=0.78) to almost the same degree, though not as well as they correlate with each other (ρ=0.93). Similarly, while cholesterol was measured with the lipid panel in the metabolomics data, it correlates with Quest (ρ=0.66) and Genova (ρ=0.65) significantly less than they correlate with each other (ρ=0.97).

Vitamin D supplementation. A common intervention for the participants was vitamin D supplementation. The Institute of Medicine has recommended a minimum 25-hydroxyvitamin D level of 20 ng/mL (Institute of Medicine (US) Committee to Review Dietary Reference Intakes for Vitamin D and Calcium, Ross, et al. (2011). Dietary Reference Intakes for Calcium and Vitamin D (Washington (DC): National Academies Press (US)).), while the Endocrine Society recommends a minimum level of 30 ng/mL (Holick, et al. (2011). J. Clin. Endocrinol. Metab. 96, 1911-1930.). At baseline, nine of the 104 individuals who were measured were below 20 ng/mL, and 45 individuals were below 30 ng/mL. After three months, two individuals were <20 ng/mL and 17 individuals were <30 ng/mL. After six months, one individual was <20 ng/mL and nine individuals were <30 ng/mL. A dose dependent effect of supplementation level (IUs) on vitamin D levels from baseline to three months was observed, with individuals taking less than 3000 IUs/day exhibiting relatively little gains in vitamin D levels. Importantly the 13 individuals that were noncompliant with the recommendations made no gains in vitamin D levels. A significant difference was observed at the 4000 and 10000 IU supplementation levels compared to the noncompliant group (FIG. 17).

Microbiome. Each participant's gut microbial community was represented as the relative abundance of operational taxonomic units (OTUs), determined from 16S rRNA sequences. The α-diversity (diversity within an individual sample) and β-diversity (diversity distinguishing two samples) was computed across rounds. α-diversity is a per-sample feature and was included in the correlation network and community analysis. In contrast, β-diversity was used to assess the degree to which each participants' microbiome composition resembled itself over time (FIG. 10. Similar to previous reports (Caporaso, et al. (2011). Genome Biol. 12, R50.; Human Microbiome Project Consortium (2012). Structure, function and diversity of the healthy human microbiome. Nature 486, 207-214.), in nearly all cases, individuals' microbiome composition was more similar to their previous sample than to other individuals.

Whether there existed an association between the Firmicutes/Bacteroidetes ratio (F/B) and obesity in the dataset was investigated. In accordance with the variable and often inconsistent findings of this ratio (Ley, (2010). Curr. Opin. Gastroenterol. 26, 5-11), no consistent and significant association between F/B and body mass index (BMI) (p<0.11) was found. Whether this ratio was associated with any other analytes measured in the study was investigated. While F/B was not specifically associated with BMI, even after stringent multiple hypothesis correction, it was significantly correlated with the abundance of numerous markers of dysbiosis in cellular energy and absorption, vitamin and toxin levels, as well as neurotransmitters (Table 8). This ratio is a coarse summary of the microbiome: it accounts for neither the presence nor the influence of taxa from other branches of the tree of life, and furthermore includes a non-specific breadth of taxonomic diversity. Nonetheless, this supports the hypothesis that the microbiome may modulate wellness via nutrient absorption, detoxification, and mental well-being via the ‘brain-gut-axis’ (Camilleri & Di Lorenzo (2012). J. Pediatr. Gastroenterol. Nutr. 54, 446-453.; Diaz et al. (2011). Proc. Natl. Acad. Sci. U.S.a. 108, 3047-3052.; Nicholson, et al. (2012). Science 336, 1262-1267), as well as the complexity involved in disentangling the web of microbe-microbe and host-microbe interactions.

TABLE 8
Analytes significantly correlated with
Firmicutes/Bacteroidetes ratio.

| Panel | Compound | rho | p< | q< |
|---|---|---|---|---|
| Cellular Energy and | Isocitric acid | 0.36 | 0.00018 | 0.016 |
| Mitochondria | Citric acid | 0.34 | 0.00049 | 0.016 |
| | Malic acid | 0.34 | 0.00061 | 0.017 |
| | Alpha ketoglutaric acid | 0.32 | 0.0012 | 0.026 |
| | Cis-aconitic acid | 0.31 | 0.0014 | 0.026 |
| | Pyruvic acid | 0.28 | 0.0042 | 0.050 |
| | Methylglutaric acid | 0.27 | 0.0060 | 0.064 |
| Malabsorption and | Phenylacetic acid | 0.34 | 0.00043 | 0.016 |
| Dysbiosis | Hippuric acid | 0.31 | 0.0018 | 0.031 |
| Markers | Benzoic acid | 0.29 | 0.0038 | 0.049 |
| | 3-hydroxyphenylacetic | 0.27 | 0.0056 | 0.063 |
| | acid | | | |
| Neurotransmitter | 3-methyl-4- | 0.33 | 0.00085 | 0.020 |
| | hydroxyphenylglycol | | | |
| | Quinolinic acid | 0.30 | 0.0023 | 0.033 |
| Toxic Elements | Cadmium | 0.36 | 0.00026 | 0.016 |
| Toxin and | Alpha ketophenylacetic | 0.34 | 0.00049 | 0.016 |
| Detoxification | acid | | | |
| Vitamin Markers | Methylmalonic acid | 0.36 | 0.00022 | 0.016 |
| | 3-hydroxypropionic acid | 0.30 | 0.0022 | 0.033 |
| | 3-hydroxyisovaleric acid | 0.29 | 0.0029 | 0.040 |
| | Alpha ketoadipic acid | 0.27 | 0.0063 | 0.064 |
| | Xanthurenic acid | 0.27 | 0.0067 | 0.064 |

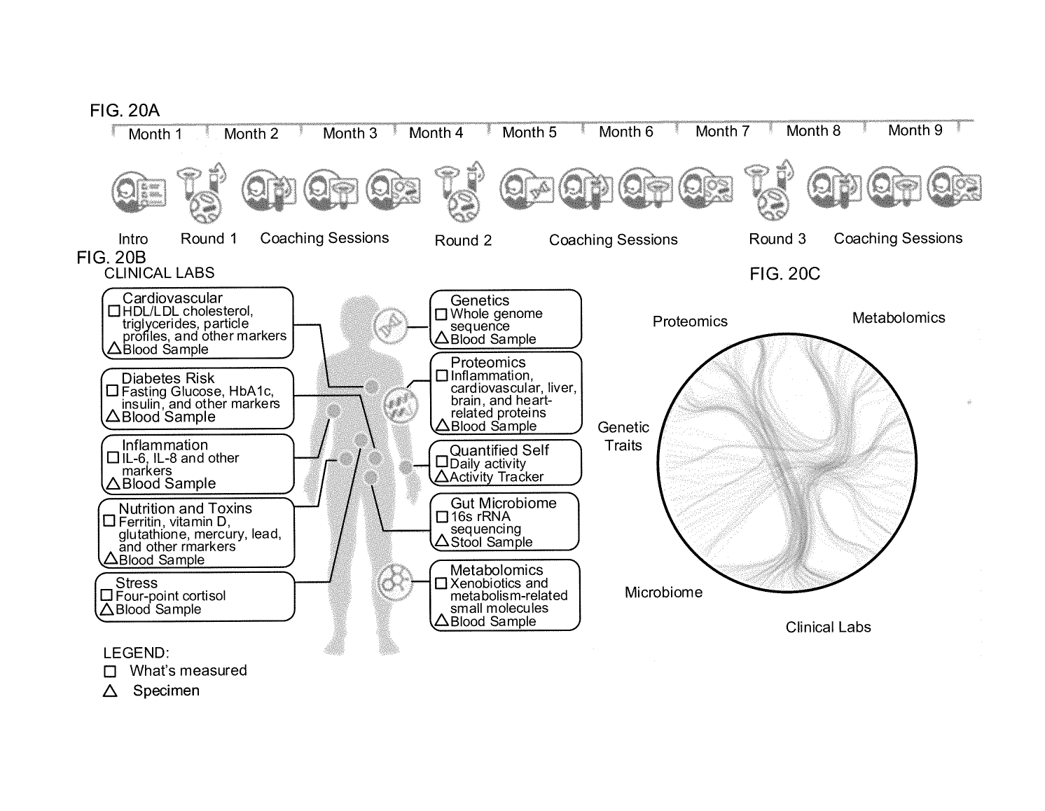

Discussion The P100 Wellness Project produced three major features. (1) Thousands of inter-omic correlations were determined and could be mapped into discrete data communities, reflecting both novel and literature-supported relationships. These correlations elucidate interactions across multi-omic data, and raised many hypotheses that could be tested by perturbation experiments. (2) Polygenic risk scores calculated from GWAS-derived common variants were predictive of quantitative traits such as levels of LDL-C, the omega-6 fatty acid DGLA, and bilirubin in this independent cohort. (3) Polygenic scores from GWAS data coupled with correlated quantitative measures of e.g. clinical laboratory tests and metabolites may suggest preventive interventions in asymptomatic individuals, for example for IBD. These multi-omic analyses embody the essence of precision medicine (Collins & Varmus (2015). N. Engl. J. Med. 372, 793-795.) and could drive the discovery of important medical applications. These are shown in FIGS. 20A-20C. In particular, FIGS. 20A-20C shows breadth of data collected longitudinally on 108 individuals and correlations across data types: (20A) Timeline of important events in the P100. (20B) Schematic of the data collected every three months throughout the study. (20C) Subset of top statistically-significant Spearman cross-sectional correlations between all datasets collected in the cohort. Each line represents one correlation. Up to 100 correlations per pair of data types are shown.

The Proteomics included in the network of FIGS. 20A-20C can include one or more of ACTA2(2), ACTA2, PTGDS, SERPINC1, GC, MBLS, HGFAC, CFHR1, CRP, CRP(2), INHBC, APCS, OSM, DNER, OPG, BEGFA, AXIN1, HGF, CDS, CXCL10, FLT3L, CCL20, TNFSF14, IL 7, IL 8, IL 18 R1, IL 10RA, FGF21, 1L6, CSF 1, SIRT2, STAMPB, 4E BP1, NCF2, PPBP, PPBP(2), BETA NGF, CSTB, VEGFA, EGF, IL 1RA, FABP4, IL 6, SIRT2, PDGF Subunit B, LEP, HSP 27, T PA, REN, CD40L, NEMO, GH.

Additionally, the Genetic Traits included in the network of FIGS. 20A-20C can include one or more of Allergic Sensitization, Deletion Cfhr1, Inflammatory bowel disease, Activated Partial Thrombastin Time, Bladder Cancer, Bilirubin Levels, Gamma Linolenic Acid, Arachidonic Acid, Dihomo Gamma Linolenic Acid, Linoleic Acid, Adrenic Acid.

The Microbiome analytes included in the network of FIGS. 20A-20C can include one or more of Coriobacteria, Deltaproteobacteria, Mollicutes, Verrucomicrobiae, Coriobacteriales, Verrucomicrobiales, Pasteurellales, Diversity, Tenericutes, Verrucomicrobia, Coriobacteriaceae, 91otu13421, Desulfovibrionaceae, Pasteurellaceae, 91otu4418, Peptostreptoccoccaceae, 91otu1825, Mogibacteriaceae, Unclassified, Christensenellaceae, Verrucomicrobiaceae.

The Clinical Labs analytes included in the network of FIGS. 20A-20C can include one or more of Alanine, Ratio Om6/Om3, Alpha Amino N Butyric Acid, Interleukin 116, Small Ldl Particle, Ratio Gln Gln, Threonine, 3 Methylhistidine, Average Inflammation Score, Mercury, Docosapentaenoic Acid, Eicosadienoic Acid, Homa Ir, Leucine, Docosatetraenoic Acid, Omega 3 Index, Tyrosine, Hdl Cholesterol, C Peptide, 1 Methyldistidine, 3 Hydroxyisovaleric Acid, Arachidonic Eicosapentaenoic, Isovalerylglycine, Isoleucine, Figlq, Palmitoleic acid, Total Cholesterol, Linoleic Dihomo γ Linoleic, Arachidonic Acid, Ldl particle, Alpha Aminoadipic Acid, Docosahexaenoic Acid, Crp Relative Risk, Total Omega 3, Valine, Phenylacetic Acid, Body Mass Index, Leptin, Weight, Height, Glutamic Acid, Hs Crp, Dihomo Gamma, Linoleic Acid, Ldl Cholesterol, Triglycerides, Insulin, Hba1c, Proinsulin, Eladic Acid, Margaric Acid, Gamma Aminobutyric Acid, Cystine, Egfr African American, Ggt, Bilirubin Total, Ldl Medium, Ldl Small, Cholesterol Total, Egfr Non African American, Alkaline Phosphatase, Bilirubin Direct, Triglycerides, Ldl Particle Number.

The Metabolomics included in the network of FIGS. 20A-20C can include one or more of TI16:0 (palmitic Acid), TI18:3n6 (g Linoleic Acid), TI15:0 (pentadecanoic Acid), TI14:1 n5 (myristoleic Acid), T20:2 n6 (eicosadienoic Acid), T20:5 n3 (eicosapentaenoic Acid), TI8:2n6 (linoleic acid), TIdm16:0 (plasmalogen Palmitic Acid), T22:6n3 (docosahexaenoic Acid), T20:3n6 (di Homo G Linoleic Acid), T22:4n6 (adrenic Acid), TI8:1n9 (oleic Acid), TIdm 18:1n9 (plsamalogen Oleic Acid), TI20:4n6 (arachidonic Acid), TI14:0 (myristic Acid), Arachidate (20:0), Stearoyl Linoleoyl Glycerophosphoethanolamine (1)*, 1 Palmitoleoylglycerophosphocholine (16:1)*, Palmitoyl Oleoyl Glycerophosphoglycerol (2)*, 1 Linoleoylglycerophosphocholine (18:2n6), Palmitoyl Linoleoyl Glycerophosphocholine (1)*, Stearoyl Arachidonoyl Glycerophosphoethanolamine (1)*, 5 Hydroxyhexanoate, 2 Hyrdoxypalmitate, Nervonoyl Sphingomyelin*, TItl (total Total Lipid), Cholesterol, Docosahexaenoate (dha; 22:6n3), Eicosapentaenoate (epa; 20:5n3), 3 Carboxy 4 Methyl 5 Propyl 2 Furanpropanoate (cmpf), 3 Methyladipate, Cholate, Phosphoethanolamine, 1 Oleoylglycerol (1 Monoolein), Tigloylglycine, Valine, Isobutyrylglycine, Isoleucine, Leucine, P Cresol Sulfate, Tyrosine, 3 Phenylpropionate (hydrocinnamate), S Methylcysteine, Cystine, N Acetyl 3 Methylhistidine*, 3 Methylhistidine, 1 Methylhistidine, N Acetyltryptophan, 3 Indoxyl Sulfate, Serotonin (5ht), Creatinine, Glutamate, Cysteine Glutathione Disulfide, Gamma Glutamylthreonine*, Gamma Glutamylalanine, Gamma Glutamylglutamate, Gamma Gluatmyltyrosine, Gamma Glutamyl 2 Aminobutyrate, Gamma Glutamylglutamine, Bradykinin, Hydroxy Pro(3), Bradykinin, Des Arg (9), Bradykinin, Mannose, Bilirubin (e,e)*, Bilverdin, Bilirubin (z,z), L Urobilin, Nicotinamide, Alpha Tocopherol, Adenosine 5′ Monophosphate (amp), 5 Acetylamino 6 Formylamino 3 Methyluracil, Hippurate, Cinnamoylglycine.

Correlation network structure across data types provides insight into human physiology. Although 108 individuals with three data collection rounds is a relatively small sample size, the integration of diverse data types generated the observation of a large number of correlations, 3,470 of which were significant after multiple hypothesis correction, including many examples of interest as discussed herein. Some of these statistical relationships, and the community structures of presumably relevant correlations that emerged around them, are supported by existing knowledge. Others provide important hypotheses for new directions in understanding human biology, the identification of biomarkers and the interrelationships of systems not previously known to be related. Two known examples demonstrate how these correlations can point towards therapeutically valuable relationships. First, community analysis identified FGF21 as a potential contributor to cardiometabolic health. Indeed, obese diabetic patients treated with an FGF21 analog have shown improvements in triglycerides and other cardiovascular markers (Gaich, et al., (2013). Cell Metab. 18, 333-340.). Second, L-thyroxine, through a negative correlation, was placed in a data community with cholesterol markers. Interestingly, supplementation with L-thyroxine lowered total cholesterol and LDL-C levels in patients with hypothyroidism in a clinical trial (Meier, et al., (2001). J. Clin. Endocrinol. Metab. 86, 4860-4866).

A novel association is the potential role of gamma-glutamyltyrosine, a metabolite of the widely used enzyme biomarker gamma-glutamyl transferase (GGT). This transferase is a clinical biomarker for liver disease, diabetes risk (Bradley et al., 2013; Lim et al., 2007) and cardiovascular disease risk (Ruttmann, et al., Circulation 112, 2130-2137). GGT catalyzes the transfer of the gamma-glutamyl moiety of glutathione to a substrate, commonly another amino acid, producing gamma-glutamyl dipeptides (Thompson & Meister (1977). J. Biol. Chem. 252, 6792-6798). One of these dipeptides, gamma-glutamyltyrosine, is highly interconnected within the cardiometabolic community and it was found that it is much more predictive of HOMA-IR (insulin resistance) than GGT; so it may be a useful diagnostic marker for diabetes risk. Indeed, in clinical studies gamma-glutamyl dipeptides also discriminate different forms of liver disease (Soga, et al., (2011). Journal of Hepatology 55, 896-905) and predict 28-day mortality in intensive care unit patients (Rogers, et al., (2014). PLoS ONE 9, e87538). These are just a few of the hundreds of community correlations that provide deeper insights into known biology or reveal novel heretofore unknown biological associations available for future explorations.

Genome disease risks, determined from GWAS data, provide an important context for the interpretation of blood measurements. A significant correlation between a genetic predisposition for high LDL-C(model based on previous studies) and the actual levels of LDL-C in the cohort was observed. This immediately suggests a testable hypothesis: individuals with similar LDL-C levels but different genetic predispositions (as determined from GWAS data) will respond differently to behavioral interventions. In particular, the risk of atherosclerosis or cardiovascular events may be more manageable by dietary or behavioral interventions in individuals with LDL-C levels higher than predicted by their genetic predisposition. A second testable hypothesis is that there are different cardiovascular risks in individuals with the same LDL-C depending on whether the level is driven more by modifiable environmental factors or lifelong genetic factors. Such genetic contexts may help differentiate individual responses to pharmaceutical intervention (e.g. statins). Development of improved risk models for clinical measurements in the context of genetic baselines presents intriguing new avenues for precision medicine. Understanding these variables and their interactions will be clinically important as cholesterol-lowering regimens and FDA drug approvals now are organized by prospective five-year risks and risk reduction (Smith & Grundy (2014). J. Am. Coll. Cardiol. 64, 601-612).

Associations between calculated polygenic risk scores derived from common GWAS variants and measured analytes were identified. Several of these were ‘positive controls’ in the sense that the observed relationship in the data occurred between an analyte (e.g. DGLA or bilirubin) and variants previously found to associate with measured levels of that analyte. Several previously unobserved genetic trait/metabolite associations were also found. For example, the genetic risk for IBD was significantly negatively correlated with levels of cystine in plasma across the cohort. In a case-control study of IBD patients with either Crohn's disease or ulcerative colitis, it was previously observed that plasma cystine and cysteine levels were abnormally low in affected individuals relative to controls, with the effect increasing with severity of the disease. Decreased availability of the limiting substrates cysteine and cystine suggests an impairment of glutathione synthesis in the intestine (Sido, et al., (1998). Gut 42, 485-492). Glutathione is an important intracellular antioxidant that is depleted in IBD inflammatory episodes, leading to excess reactive oxygen species (ROS) and subsequent colonic inflammation and oxidative damage. Although Sido et al. discuss cystine deficiency as an effect rather than a cause of IBD, the result suggests that lower levels of blood cystine may be more common in individuals at higher genetic risk for IBD throughout their lives well before the disease manifests itself. Individual longitudinal data of the type described herein can help differentiate whether particular biomarkers found in case/control studies correlate with disease, with disease predisposition, or with both.

Plasma AFMU levels were also identified as a covariate of bladder cancer risk. The metabolite AFMU is known as a probe to measure NAT2 activity in urine after administration of caffeine (Miners & Birkett (1996). General Pharmacology: the Vascular System 27, 245-249). The slow acetylation NAT2 phenotype is associated with increased bladder cancer risk (Okkels, et al., (1997). Cancer Epidemiol. Biomarkers Prey. 6, 225-231). Importantly, the correlation between AFMU levels and bladder cancer risk is detected without administration of caffeine or clinical evidence of bladder cancer.

Traditionally, specific genetic variants have been used to explain metabolite profiles using targeted variant-pathway interactions (Guo, et al., (2015). Proc. Natl. Acad. Sci. U.S.a. 112, E4901-E4910.). The data suggest that GWAS polygenic risk scores can identify analyte associations with disease risk in a non-targeted manner (e.g. AFMU vs. bladder cancer) and in the absence of direct associations between GWAS loci and plausible metabolic pathways (e.g. cystine vs. IBD). Perhaps supplementation in healthy individuals with high IBD genetic risk could avoid the long-term low grade inflammation and oxidative damage—and thus avoid the wellness to disease transition to IBD.

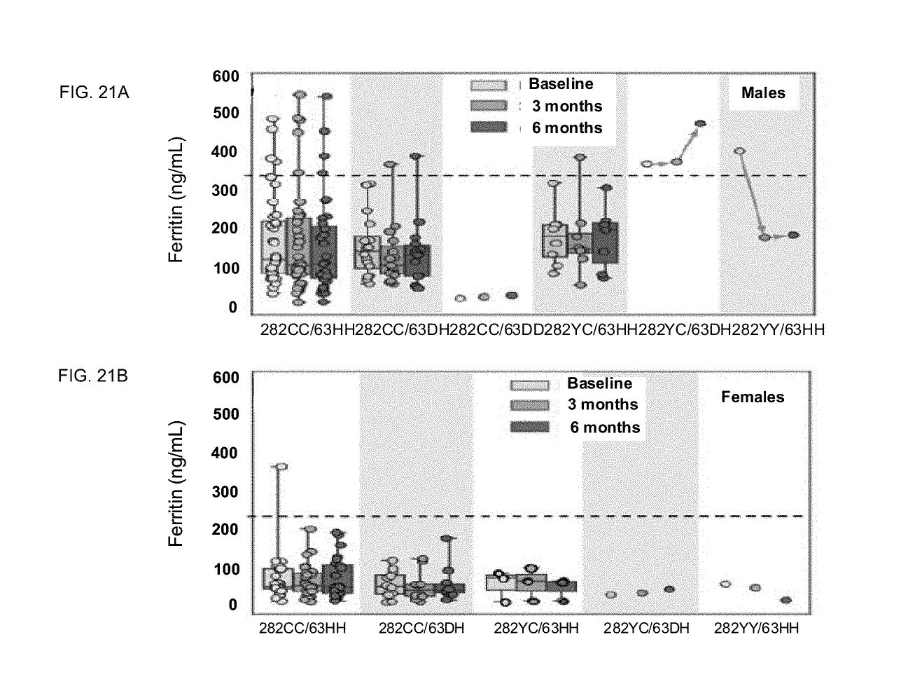

Multi-omic longitudinal data enable early intervention to promote wellness and reverse early disease. The opportunities for observing dramatic health transitions in a cohort of 108 individuals over nine months are limited, and it is unlikely that statistical significance will be reached in changes that occur in only a few individuals. For this reason, extending this pilot program to very large populations is envisioned (Hood & Price (2014). Sci Transl Med 6, 225ed5-225ed5.). Nevertheless, many clinical improvements in the participants were observed throughout the nine-month study as shown in Table 5 above. From the baseline blood collection six individuals presented with ferritin levels considerably higher than the normal upper reference range (345 ng/mL in males and 232 ng/mL in females). One participant, a 65-year old male, had ferritin levels of 399 ng/mL (2.1 SD above the mean for males) and was homozygous for HFE C282Y, the primary genetic risk factor for hereditary hemochromatosis (HH). He was referred to a hematologist, who diagnosed HH and prescribed therapeutic phlebotomy. At the next blood draw, his ferritin levels had dropped to 175 ng/mL (0.05 SD above the mean for males) and they remained normal throughout the remainder of the study (FIGS. 21A (males) and 21B (females)). HH leads to excessive accumulation of dietary iron in various tissues and can be associated with serious complications later in life, including liver disease, diabetes, arthritis, and cardiac decompensation. The early reversal of this disease can greatly reduce lifetime healthcare expenditures. FIGS. 21A and 21B show genetic risk factors for hemochromatosis. Boxplots for ferritin levels of the male and female participants by round. Only one male in the study was homozygous for 282YY and diagnosed with hemochromatosis after physician referral. Changes in ferritin levels is shown by the red arrows. A second male who was compound heterozygous for 282YC/63DH did not receive therapeutic phlebotomy, and ferritin levels increased. Several other males presented with elevated ferritin levels but none of the common genetic risk factors; they were referred to their physician for monitoring.

Statistically significant improvements in: prediabetes markers, by enhanced exercise and dietary regimes; nutritional markers, including vitamin D following preliminary genetic risk assessment and subsequent supplementation (FIG. 17); and toxic metal exposure, including mercury by reduced consumption of certain seafood or replacement of dental fillings (see Table 5) were observed.

To further explore the associations between genetic risk for biological conditions and dynamic analytes, a polygenic risk score for age-associated Alzheimer's disease (e.g. using polygenic risk score calculation module 312) was computed. This polygenic risk score was computed using a data set of published associations between individual genetic variants and risk for Alzheimer's disease from the International Genomics of Alzheimer's Project (IGAP). This score was computed for more than 2000 people for whom whole genome sequence data were available. Importantly, these individuals have not been diagnosed with Alzheimer's disease.

This polygenic risk score for age-associated Alzheimer's disease was compared against a set of 276 proteins measured from blood plasma in the same individuals using linear regression. The linear regression model included an interaction term between age and genetic risk for Alzheimer's. Results were analyzed to identify proteins that exhibited a significant interaction between age and genetic risk. In other words, the genetic risk of each individual had an impact on how the protein changed with age. Table 9 shows the analytes for which genetic risk for Alzheimer's disease modifies the relationship between the analyte and age (interaction effect).

TABLE 9
Analytes significantly correlated with
genetic risk for Alzheimer's disease.

| Type | Phenotype | P-value |
|---|---|---|
| Protein | MMP2 | 5.4e−4 |
| Protein | CDH5 | 7.3e−4 |
| Metabolite | Behenoylcarnitine | 1.6e−3 |
| Metabolite | Arachidoylcarnitine | 2.3e−3 |
| Protein | PLAU | 2.9e−3 |

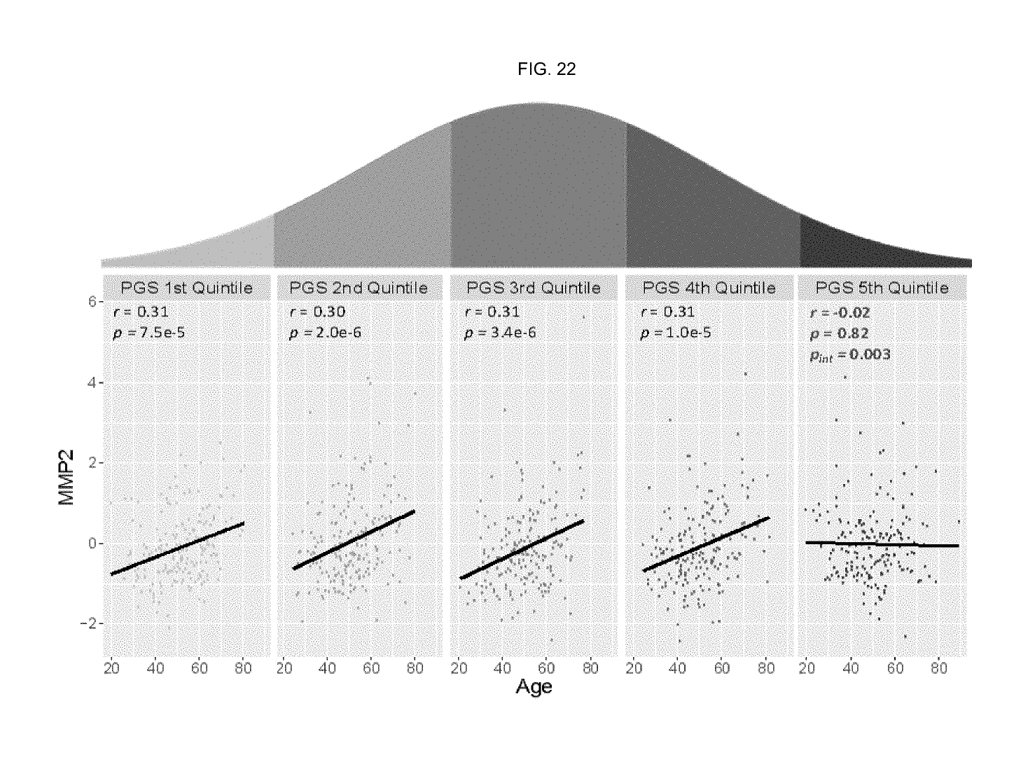

The results are shown in FIG. 22. The genetic risk for Alzheimer's disease is shown binned into quintiles (each bin contains 20% of the individuals tested), with those in the lowest genetic risk for Alzheimer's disease (as determined by the polygenic risk score) on the left, and those with the highest genetic risk for Alzheimer's disease on the right. The relationship between the most statistically significant protein (MMP2) and age for each quintile is shown as a series of scatterplots. Focusing on the left-most plot, it is observed that MMP2 increases with age. The correlation coefficient for the relationship between age and MMP2 levels is 0.31 (p=7.5e-5). The relationship is similar for the next three quintiles: as age increases, MMP2 increases, with very similar correlation coefficients. However, for those individuals at the highest genetic risk for Alzheimer's disease (in the right-most scatterplot), MMP2 does not increase with age, instead remaining fairly constant with age (correlation coefficient=−0.02, p=0.82).

This observation enables the formulation of a hypothesis regarding levels of MMP2 and genetic risk for Alzheimer's disease. It is known from the literature that individuals with Alzheimer's disease exhibit decreased clearance of amyloid β (Aβ) in their central nervous system (Mawuenyega et al., 2010). MMP2 degrades the fibrillar form of amyloid β, and is normally upregulated in response to increases in Aβ (Nalivaeva, Beckett, Belyaev, & Turner, 2012) in a potential feedback mechanism. By some unknown mechanism, being at higher genetic risk for Alzheimer's disease prevents MMP2 from expressing at higher levels as individuals age, thus reducing their ability to clear Aβ and increasing their risk for developing Alzheimer's disease. Therefore, this represents an example of the manifestation of genetic risk in the body, and suggests a preventative pharmacointervention (upgregulation of MMP2) for those at high risk of developing this disease.

Comprehensive studies of wellness are lacking and the field's reputation has yet to be established. In addition to helping individuals achieve their individual potential, “scientific wellness” will enable 1) the identification of the earliest biological network perturbations that lead to common diseases, 2) the design of diagnostics to detect these early disease transitions, and 3) development of drugs and other interventions to reverse individuals from early transitions to disease back to health. Dealing with these earliest disease transitions is the key to both predictive and preventive medicine for the individual. Moreover, these studies support the conclusion that the health-informed individual can make many decisions central to her or his own healthcare. These principles are the essence of predictive, preventive, personalized, and participatory (P4) medicine.

The operations of the example processes are illustrated herein in individual blocks and summarized with reference to those blocks. The processes are illustrated as logical flows of blocks, each block of which can represent one or more operations that can be implemented in hardware, software, or a combination thereof. In the context of software, the operations represent computer-executable instructions stored on one or more computer-readable media that, when executed by one or more processors, enable the one or more processors to perform the recited operations. Generally, computer-executable instructions include routines, programs, objects, modules, components, data structures, and the like that perform particular functions or implement particular abstract data types. The order in which the operations are described is not intended to be construed as a limitation, and any number of the described operations can be executed in any order, combined in any order, subdivided into multiple sub-operations, and/or executed in parallel to implement the described processes. The described processes can be performed by resources associated with computing system 118 such as one or more internal or external CPUs or GPUs, and/or one or more pieces of hardware logic such as FPGAs, DSPs, or other types of accelerators.

All of the methods and processes described above may be embodied in, and fully automated via, specialized computer hardware. Some or all of the methods may alternatively be embodied in software code modules executed by one or more general purpose computers or processors. The code modules may be stored in any type of computer-readable storage medium or other computer storage device.

Any routine descriptions, elements or blocks in the flow diagrams described herein and/or depicted in the attached figures should be understood as potentially representing modules, segments, or portions of code that include one or more executable instructions for implementing specific logical functions or elements in the routine. Alternate implementations are included within the scope of the examples described herein in which elements or functions may be deleted, or executed out of order from that shown or discussed, including substantially synchronously or in reverse order, depending on the functionality involved as would be understood by those skilled in the art. It should be emphasized that many variations and modifications may be made to the above-described examples, the elements of which are to be understood as being among other acceptable examples. All such modifications and variations are intended to be included herein within the scope of this disclosure and protected by the following claims.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### ADDITIONAL REFERENCES

Diamandis (2015). BMC Med 13, 5.
Dixon (2003). Journal of Vegetation Science 14, 927-930.
Erickson, et al., (2012). Integrated metagenomics/metaproteomics reveals human host-microbiota signatures of Crohn's disease. PLoS ONE 7, e49138.
Farmer, et al. (2004). Neuroscience 124, 71-79.
Ferrannini, et al. (2013). Diabetes 62, 1730-1737.
Ferris, et al. (2007). Med Sci Sports Exerc 39, 728-734.
Hood & Friend (2011). Nat Rev Clin Oncol 8, 184-187.
Hood, et al., (2004). Science 306, 640-643.
Hood, et al., (2015). BMC Med 13, 4.
Kidd, et al., (2016). J Clin Invest 126, 1734-1744.
Knowler, et al., N. Engl. J. Med. 346, 393-403.
Ley, et al. (2006). Nature 444, 1022-1023.
Mantel (1967). Cancer Res. 27, 209-220.???
Micheel, et al., (2012). Evolution of Translational Omics: Lessons Learned and the Path Forward (Washington (DC): National Academies Press (US)).
Perry, et al., (1998). Diabetes Care 21, 732-737.
Qin, et al., (2010). Nature 464, 59-65.
Rothman, et al., (2010). Nat. Genet. 42, 978-984.
Schloss, et al., (2009). Appl. Environ. Microbiol. 75, 7537-7541.
Szuhany, et al. (2015). J Psychiatr Res 60, 56-64.
Turnbaugh, et al. (2009). Nature 457, 480-484.
Wahl, et al. (2015). BMC Med 13, 48.
Wang, et al., (2007). Appl. Environ. Microbiol. 73, 5261-5267.
Wannamethee, et al., (1995). Am. J. Epidemiol. 142, 699-708.
Yong, et al., (2010). The Healthcare Imperative: Lowering Costs and Improving Outcomes: Workshop Series Summary (Washington (DC): National Academies Press (US)).

1. A computing system comprising:
one or more processors; and
non-transitory memory including computer-readable instructions that when executed by the one or more processors perform operations comprising:
obtaining clinical testing data for a plurality of clinical tests;
obtaining biological data for a plurality of individuals, the biological data including a plurality of dynamic analytes;
analyzing the clinical testing data and the biological data to determine respective correlations between at least one of (1) pairs of clinical tests of the plurality of clinical tests, (2) pairs of dynamic analytes of the plurality of dynamic analytes, or (3) pairs of a respective clinical test of the plurality of clinical tests and a respective dynamic analyte of the plurality of dynamic analytes;
generating a network of correlations that includes at least a portion of the respective correlations, the network of correlations including vertices and edges between respective pairs of the vertices, each vertex of the vertices corresponding to a clinical test of the plurality of clinical tests or a dynamic analyte of the plurality of dynamic analytes, and an edge of the edges indicates a statistical correlation between a pair of vertices;
determining that an individual of the plurality of individuals is asymptomatic with respect to a biological condition;
performing an analysis of the biological data of the individual with respect to the network of correlations; and
determining a probability the individual will exhibit one or more phenotypes of the biological condition based at least partly on the analysis.

2. The computing system of claim 1, wherein the operations further comprise:
determining that the probability the individual will exhibit the one or more phenotypes of the biological condition is greater than a threshold probability; and
determining an intervention from a plurality of interventions, wherein the intervention is designed to reduce the probability that the individual will exhibit the one or more phenotypes of the biological condition.

3. The computing system of claim 1, wherein the statistical correlation between the pair of vertices is above a threshold.

4. The computing system of claim 1, further comprising a data store storing the clinical testing data and the biological data for the plurality of individuals in the data store.

5. The computing system of claim 1, wherein the biological data includes genomic data, proteomic data, metabolomics data, gut microbiome data, or combinations thereof.

6. The computing system of claim 1, wherein the plurality of dynamic analytes include one or more metabolites, one or more proteins, at least a portion of respective genomes of the plurality of individuals, at least a portion of respective microbiomes of the plurality of individuals, or combinations thereof.

7. The computing system of claim 1, wherein the operations further comprise determining a group including at least one of (1) a plurality of vertices of the network of correlations related to the biological condition or (2) a plurality of edges of the network of correlations related to the biological condition.

8. The computing system of claim 1, wherein the analysis is performed based at least partly on a hierarchical level of the network of correlations determined based at partly on a modularity of the network of correlations, the modularity corresponding to an arrangement of a number of edges of the network of correlations that is statistically improbable in relation to an equivalent network of correlations with edges placed at random.

9. The computing system of claim 1, wherein the analysis of the network of correlations includes determining Spearman's ρ for at least one of (1) one or more pairs of clinical tests selected from the plurality of clinical tests, (2) one or more pairs of dynamic analytes selected from the plurality of dynamic analytes, or (3) one or more pairs including a respective clinical test selected from the plurality of clinical tests and a respective dynamic analyte selected from the plurality of dynamic analytes.

10. The computing system of claim 1, wherein the analysis of the network of correlations includes: for individual edges of the network of correlations, calculating a number of weighted shortest paths from all vertices to all other vertices that pass over an individual edge and removing edges that are associated with at least a threshold number of weighted shortest paths.

11. A computer-implemented method comprising:
obtaining, by a computing device including a processor and memory, clinical testing data for a plurality of clinical tests;
obtaining, by the computing device, biological data for a plurality of individuals, the biological data including a plurality of dynamic analytes and the plurality of individuals are asymptomatic with respect to a biological condition;
analyzing, by the computing device, the clinical testing data and the biological data to determine respective correlations between at least one of (1) pairs of clinical tests of the plurality of clinical tests, (2) pairs of dynamic analytes of the plurality of dynamic analytes, or (3) pairs of a respective clinical test of the plurality of clinical tests and a respective dynamic analyte of the plurality of biological indicators;
generating, by the computing device, a network of correlations that includes at least a portion of the respective correlations, the network of correlations including vertices and edges between respective pairs of the vertices, each vertex of the vertices corresponding to a clinical test of the plurality of clinical tests or a dynamic analyte of the plurality of dynamic analytes, and an edge of the edges indicates a statistical correlation between a pair of vertices;
determining, by the computing device, a number of pre-existing dynamic analytes for the biological condition based at least partly on data obtained from an additional plurality of individuals that exhibited one or more phenotypes of the biological condition; and
determining, by the computing device, one or more additional dynamic analytes for the biological condition based at least partly on the network of correlations.

12. The method of claim 11, wherein the one or more additional dynamic analytes are not associated with the biological condition in previously published literature and the pre-existing dynamic analytes are included in the previously published literature.

13. The method of claim 11, further comprising determining one or more parameters for a clinical trial regarding an intervention for the biological condition, wherein the intervention regulates the one or more additional dynamic analytes.

14. The method of claim 11, wherein the biological condition is Alzheimer's disease and the one or more additional dynamic analytes include matrix metalloproteinase-2 (MMP2) and the one or more pre-existing dynamic analytes include amyloid β.

15. The method of claim 11, wherein the biological condition is metabolic syndrome and the one or more additional dynamic analytes includes gamma-glutamyltyrosine.

16.-20. (canceled)

21. The computing system of claim 7, wherein:
the group includes a plurality of sub-groups and the group corresponds to cardiovascular health; and
a sub-group of the plurality of sub-groups includes a portion of the plurality of vertices of the network of correlations and a portion of the plurality of edges of the network of correlations, the sub-group corresponding to total cholesterol and low-density lipoprotein (LDL) cholesterol.

22. The method of claim 11, further comprising:
determining, by the computing device, a plurality of effect alleles of the biological condition, each effect allele of the plurality of effect alleles including a single nucleotide variant (SNV);
determining, by the computing device, an effect size for an effect allele of the plurality of effect alleles, the effect size of the effect allele corresponding to a contribution of a SNV of the effect allele to one or more genetic variations related to the biological condition;
determining, by the computing device, an allele score for the individual with respect to the biological condition based at least partly on the effect allele carried by the individual and the effect size of the effect allele;
determining, by the computing device, a sum of the allele score and a plurality of additional allele scores to determine a polygenic risk score, the plurality of additional allele scores being calculated based on (1) additional effect alleles of the plurality of effect alleles and (2) respective additional effect sizes of the additional effect alleles; and
calculating, by the computing device, a genetic risk of the biological condition for the individual based at least partly on the polygenic risk score.

23. A method comprising:
receiving multi-omic data from a population, wherein the multi-omic data comprises genomic data, and at least one of proteomic data, metabolomic data, microbiome data, transcriptomics data, epigenomic data, or clinical test results data;
calculating genetic risk for a plurality of conditions for the plurality of individuals utilizing the genomic data and genome-wide association studies (GWAS);
normalizing and transforming the genetic risk and the multi-omic data into comparable data vectors;
statistically analyzing the comparable data vectors and the genetic risk for the plurality of conditions to determine a network of correlations, the network of correlations including vertices and edges between respective pairs of the vertices, each vertex of the vertices corresponding to a clinical test or a dynamic analyte included in the multi-omic data, and an individual edge of the edges indicating a statistical correlation between a pair of vertices; and
determining, based at least partly on the network of correlations, that a dynamic analyte is present in one or more individuals included in the population before a condition of the plurality of conditions emerges in the one or more individuals.

24. The method of claim 23, wherein:
the dynamic analyte is correlated with a genetic risk for the condition and the dynamic analyte is indicative of the genetic risk before the condition emerges in the individual, wherein the dynamic analyte was not previously known to correlate with the genetic risk for the condition in the GWAS; or
up- or down-regulation of the dynamic analyte occurs before the condition emerges in the individual and the up- or down-regulation of the dynamic analyte was previously only known to be associated with the condition after the condition had developed in individuals.

25. The method of claim 23, further comprising excluding one or more additional GWAS from the GWAS based on one or more of: a sample size of less than 5000 individuals for the one or more additional GWAS; description of genetic risk associated with less than 5 or fewer single nucleotide variants (SNVs) associated with the one or more additional GWAS; or lack of at least one SNV with a p-value of <10-e8 for the one or more additional GWAS.
